SEQUENCE LISTING (13 sequences)

SEQ ID NO: 1 - DNA, 58 bp, Artificial
  shRNA against ITGBeta1
ccgggccttgcattactgctgatatctcgagatatcagcagtaatgcaaggctttttg

SEQ ID NO: 2 - DNA, 21 bp, Artificial
  oligonucleotide primer
tgtctgatcttgctaggaccg

SEQ ID NO: 3 - DNA, 22 bp, Artificial
  oligonucleotide primer
gagagtaacggcctttttgtga

SEQ ID NO: 4 - DNA, 23 bp, Artificial
  oligonucleotide primer
atccacggcatactatcaacatc

SEQ ID NO: 5 - DNA, 21 bp, Artificial
  oligonucleotide primer
caaggctcaccatcatcgtag

SEQ ID NO: 6 - DNA, 23 bp, Artificial
  oligonucleotide primer
ccctcacactcagatcatcttct

SEQ ID NO: 7 - DNA, 19 bp, Artificial
  oligonucleotide primer
gctacgacgtgggctacag

SEQ ID NO: 8 - DNA, 19 bp, Artificial
  oligonucleotide primer
gctcctcttaggggccact

SEQ ID NO: 9 - DNA, 19 bp, Artificial
  oligonucleotide primer
ccacgtctcaccattgggg

SEQ ID NO: 10 - DNA, 20 bp, Artificial
  oligonucleotide primer
ctccatcgtcatcatcatcg

SEQ ID NO: 11 - DNA, 20 bp, Artificial
  oligonucleotide primer
tgcacccagatcctatagcc

SEQ ID NO: 12 - DNA, 20 bp, Artificial
  oligonucleotide primer
cgcttccttacctggttgat

SEQ ID NO: 13 - DNA, 20 bp, Artificial
  oligonucleotide primer
gagcgaccaaaggaaccata

(19) United States
(12) Patent Application Publication
(10) Pub. No.: US 2022/0220206 A1
(43) Pub. Date: Jul. 14, 2022

(54) TREATING CHRONIC LIVER DISEASE

(71) Applicant: Mayo Foundation for Medical Education and Research, Rochester, MN (US)

(72) Inventor: Samar H. Ibrahim, Rochester, MN (US)

(21) Appl. No.: 17/612,286

(22) PCT Filed: May 20, 2020
(86) PCT No.: PCT/US2020/033726

§ 371 Date: Nov. 18, 2021

Related U.S. Application Data
(60) Provisional application No. 62/889,686, filed on Aug. 21, 2019.
(60) Provisional application No. 62/850,388, filed on May 20, 2019.

(51) Int. Cl. C07K 16/28; A61P 1/16

(52) U.S. Cl. CPC C07K 16/2842; A61P 1/16; A61K 2039/505; C07K 2317/76; C07K 16/2836

(57) ABSTRACT

This document provides methods and materials involved in treating chronic liver disease (e.g., non-alcoholic fatty liver disease such as nonalcoholic steatohepatitis (NASH)). For example, methods and materials for using one or more inhibitors of an integrin β1 (ITGβ1) polypeptide, one or more inhibitors of an integrin α9 polypeptide (ITGα9), and/or one or more inhibitors of a vascular cell adhesion molecule 1 (VCAM-1) polypeptide to treat a mammal having chronic liver disease are provided.

### CROSS-REFERENCE TO RELATED APPLICATIONS

This application claims the benefit of U.S. Patent Application Ser. No. 62/850,388, filed on May 20, 2019, and claims the benefit of U.S. Patent Application Ser. No. 62/889,686, filed on Aug. 21, 2019. The disclosure of the prior application is considered part of (and is incorporated by reference in) the disclosure of this application.

### STATEMENT REGARDING FEDERAL FUNDING

This invention was made with government support under DK111397 awarded by the National Institutes of Health. The government has certain rights in the invention.

### BACKGROUND

### 1. Technical Field

This document relates to methods and materials involved in treating chronic liver disease (e.g., non-alcoholic fatty liver disease such as nonalcoholic steatohepatitis (NASH)). For example, one or more inhibitors of an integrin β1 (ITGβ1) polypeptide, one or more inhibitors of an integrin α9 polypeptide (ITGα9), and/or one or more inhibitors of a vascular cell adhesion molecule 1 (VCAM-1) polypeptide can be administered to a mammal (e.g., a human) having a chronic liver disease to treat the mammal.

### 2. Background Information

Nonalcoholic fatty liver disease (NAFLD) is currently the most common chronic liver disease (Bellentani et al., Liver Int, 37(Suppl 1):81-84 (2017)). A subset of patients with NAFLD develops NASH, a more severe inflammatory form of NAFLD, which can progress to end-stage liver disease. NASH is currently the leading cause of liver-related mortality in many western countries (Younossi et al., J. Hepatol., 70(3):531-544 (2019)).

### SUMMARY

There is an unmet need for mechanism-based therapeutic strategies that can reverse established NASH and control or reduce the progression of NASH to end-stage liver disease (Friedman et al., Nat. Med., 24(7):908-922 (2018)).

This document provides methods and materials involved in treating chronic liver disease (e.g., non-alcoholic fatty liver disease such as NASH). For example, one or more inhibitors of an ITGβ1 polypeptide, one or more inhibitors of an ITGα9 polypeptide, and/or one or more inhibitors of a VCAM-1 polypeptide can be administered a mammal (e.g., a human) having a chronic liver disease to treat the mammal.

Integrins (ITGs) are heterodimeric cell surface transmembrane proteins with a non-covalently associated α subunit and β subunit that mediate cell-cell and cell-matrix interactions (Hynes et al., Cell, 110(6):673-87 (2002)). As described herein, an integrin heterodimer including an ITGIβ1 polypeptide subunit and an ITGα9 polypeptide subunit (an ITGα9β₁ heterodimer) is involved in the progression of NASH. ITGα9β₁ heterodimers are released from hepatocytes under lipotoxic stress as a cargo of extracellular vesicles (EVs), and signaling initiated from an ITGα9β1 heterodimer interacting with a VCAM-1 ligand mediates monocyte adhesion to liver sinusoidal endothelial cells and can lead to hepatic inflammation. In a mouse model of NASH, blocking ITGβ1 via anti-ITGβ1 antibody treatment was confirmed to reduce liver inflammation, injury, and fibrosis.

Having the ability to reduce or eliminate signaling initiated from an ITGα9β1 heterodimer interacting with a VCAM-1 ligand using one or more inhibitors of an ITGβ1 polypeptide, one or more inhibitors of an ITGα9 polypeptide, and/or one or more inhibitors of a VCAM-1 polypeptide provides a unique and unrealized opportunity to treat chronic liver disease such as NASH.

In general, one aspect of this document features methods for treating chronic liver disease in a mammal. The methods can include, or consist essentially of, administering an inhibitor of an ITGβ1 polypeptide, an inhibitor of an ITGα9, or an inhibitor of a VCAM-1 polypeptide to said mammal. The mammal can be a human. The chronic liver disease can be a non-alcoholic fatty liver disease. The non-alcoholic fatty liver disease can be NASH. In cases where the method includes administering an inhibitor of an ITGβ1 polypeptide to the mammal, the inhibitor of an ITGβ1 polypeptide can be an ITGβ1 neutralizing antibody. In cases where the method includes administering an inhibitor of an ITGα9 polypeptide to the mammal, the inhibitor of an ITGα9 polypeptide can be an ITGα9 neutralizing antibody. In cases where the method includes administering an inhibitor of a VCAM-1 polypeptide to the mammal, the inhibitor of a VCAM-1 polypeptide can be a VCAM-1 neutralizing antibody. The method also can include, before the administering step, identifying the mammal as being in need of treatment of the chronic liver disease.

In another aspect, this document features a method for reducing liver inflammation in a mammal having chronic liver disease. The methods can include, or consist essentially of, administering an inhibitor of an ITGβ1 polypeptide, an inhibitor of an ITGα9 polypeptide, or an inhibitor of a VCAM-1 polypeptide to the mammal. The mammal can be a human. The chronic liver disease can be a non-alcoholic fatty liver disease. The non-alcoholic fatty liver disease can be NASH. In cases where the method includes administering an inhibitor of an ITGβ1 polypeptide to the mammal, the inhibitor of an ITGβ1 polypeptide can be an ITGβ1 neutralizing antibody. In cases where the method includes administering an inhibitor of an ITGα9 polypeptide to the mammal, the inhibitor of an ITGα9 polypeptide can be an ITGα9 neutralizing antibody. In cases where the method includes administering an inhibitor of a VCAM-1 polypeptide to the mammal, the inhibitor of a VCAM-1 polypeptide can be a VCAM-1 neutralizing antibody. The method also can include, before said administering step, identifying the mammal as being in need of the reduced liver inflammation.

In another aspect, this document features a method for reducing liver fibrosis in a mammal having chronic liver disease. The methods can include, or consist essentially of, administering an inhibitor of an ITGβ1 polypeptide, an inhibitor of an ITGα9 polypeptide, or an inhibitor of a VCAM-1 polypeptide to the mammal. The mammal can be a human. The chronic liver disease can be a non-alcoholic fatty liver disease. The non-alcoholic fatty liver disease can be NASH. In cases where the method includes administering an inhibitor of an ITGβ1 polypeptide to the mammal, the inhibitor of an ITGβ1 polypeptide can be an ITGβ1 neutralizing antibody. In cases where the method includes administering an inhibitor of an ITGα9 polypeptide to the mammal, the inhibitor of an ITGα9 polypeptide can be an ITGα9 neutralizing antibody. In cases where the method includes administering an inhibitor of a VCAM-1 polypeptide to the mammal, the inhibitor of a VCAM-1 polypeptide can be a VCAM-1 neutralizing antibody. The method also can include, before said administering step, identifying the mammal as being in need of the reduced liver fibrosis.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Lipotoxic hepatocyte-derived EVs are enriched with active ITGβ₁. (A) Top ranked canonical pathways identified by Ingenuity Pathway Analysis (IPA) of proteomic data on EVs derived from vehicle or lysophosphatidylcholine (LPC)-treated primary mouse hepatocytes (PMH). Immunoblot analysis showing protein levels of integrin family members and Talin-1 on EVs and whole cell lysate (WCL) from PMH (B) or Huh7 cells (C) treated with vehicle or 20 μM LPC for 4 hours. Beta-actin, and the EV markers TSG101, CD63 and CD81 were used as loading controls for WCL and EVs, respectively. (D) Immunogold electron microscopy images showing immunoreactivity for ITGβ1 in an active conformation on EVs derived from PMH treated with vehicle (Veh-EV) or LPC (LPC-EV). (E) Nanoscale flow cytometry showing expression levels of active ITGβ1 on EVs. Silica nanoparticles of various sizes are used as calibration beads and EVs were defined based on the particle size (Top panel). ITGβ₁-positive EVs from PMH treated with Veh or LPC (Bottom panel). Quantification of ITGβ₁-positive EVs from (F) PMH and (G) Huh7. Bar columns represent mean±standard error of the mean (SEM); n=3-5. (H) Quantification of ITGβ1⁺ EVs in the serum of patients with simple steatosis (n=8), and NASH with stage 1-2 fibrosis (n=17). Plots represent mean±SEM; *p<0.05, **p<0.01.

FIG. 2. Hepatocyte lipotoxic treatment induces ITGβ₁ activation and endocytic trafficking. (A) Schematic representation of activation and endocytic trafficking of ITGβ₁. (B) Huh7 cells and (C) AML12 cells were treated with either vehicle or 20 μM LPC for 15-30 min with or without 10 μM p38 inhibitor SB203580 (SB). Cell lysates were immunoprecipitated with active conformation-sensitive ITGβ₁ antibody (9EG7), inactive conformation-sensitive ITGβ₁ antibody (Mab13) or isotype IgG. Immunoblot was performed using a total anti-ITGβ₁ antibody. Beta-actin was used as a loading control for input samples. Huh7 cells were treated with either vehicle or 5 μM LPC for 20 minutes with or without 10 μM SB203580, (D) active ITGβ₁ was labeled with 9EG7. (E) Co-localization of active ITGβ₁ (9EG7) with early endosomes, late endosomes, or MVBs was assessed using anti-EEA1, anti-Rab7, and anti-CD63 antibodies, respectively. Scale bar: 5 μm. Pearson's correlation coefficient of co-localization was employed to quantify the co-localization between two fluorophores.

FIG. 3. Active ITGβ₁ is not targeted for lysosomal degradation in lipotoxic hepatocytes: Huh7 cells were treated with either vehicle or 10 μM LPC for 20 minutes. Active ITGβ₁ and lysosomes were labeled with 9EG7 antibody and anti-LAMP1 antibody, respectively. Pearson's correlation coefficient was employed to quantify the degree of co-localization between the two fluorophores. Scale bar: 5 μm, (ns, nonsignificant).

FIG. 4. Fat, fructose, and cholesterol (FFC)-fed mice have increased circulating EVs of hepatocyte origin. (A) Schema of a mouse model to track circulating EVs of hepatocyte origin. (B) Number of GFP-positive events (EV of hepatocyte origin)/1 μL of plasma of Gt (ROSA)26Sor^{tm4(ACTB-tdTomato,-EGFP)Luo}/J chow-fed mice (Control), AAV8.TBG.PI.Cre.rBG-injected chow-fed mice (Cre-chow), and AAV8.TBG.PI.Cre.rBG-injected FFC-fed mice for 24 weeks (Cre-FFC) as measured by nanoscale flow cytometry. *p<0.05; n=6 per each group.

FIG. 5. Lipotoxic hepatocyte-derived EVs promote monocyte adhesion to liver sinusoidal endothelial cells (LSECs) via an ITGβ₁-dependent mechanism. (A) Primary mouse monocytes were treated with EVs from LPC (LPC-EV)- or vehicle (Veh-EV)-treated PMH, total RNA was isolated, submitted for RNA sequencing, and analyzed by IPA, showing the top represented canonical pathways in monocytes stimulated with LPC-EVs vs Veh-EVs. (B) Equal number of Huh7 cells were treated with either vehicle or LPC. EVs were collected from the conditioned media and labelled with DiO. THP1 cells were co-cultured with human LSECs in the presence of DiO-labelled LPC-EV, or Veh-EV. Scale bar: 20 μm for the top panel, and 5 μm for the bottom panel. (C) Z-stack confocal microscopy of THP1 incubated with DiO-labelled EVs from LPC-treated Huh7 cells (white arrows). (D) Primary mouse monocytes were stimulated with Veh-EV or LPC-EV from PMH±ITGβ₁Ab, and infused in microfluidic chambers coated with a monolayer of primary mouse LSECs. Adherent cells were quantified. (E) Immunoblot analysis showing ITGβ₁ knockdown in shiTGβ₁ cell line. Beta-actin was used as a loading control. (F) THP1 cells were stimulated with either Veh-EV or LPC-EV from wild-type (WT) Huh7 cells, or from shiTGβ₁ Huh7 cells, and infused in microfluidic chambers coated with a monolayer of primary human LSECs±VCAM-1 Ab. Adherent THP1 cells were quantified similar to D. VCAM-1 is expressed on human LSECs under basal condition as shown by flow cytometry; n=6.

FIG. 6. LPC-EVs enhance monocyte adhesion to LSEC via an ITGβ1-VCAM1 interaction. (A) Diagram of the microfluidic device. (B) Photo of the microfluidic device employed in the adhesion assay. (C) Representative pictures of adherent THP1 cells stimulated with EVs from the different experimental conditions to primary human LSECs. Scale bars: 500 (D) THP1 cells were stimulated with either Veh-EV or LPC-EV from Huh7 cells±ITGα₉ neutralizing Ab, and infused in microfluidic chambers coated with a monolayer of primary human LSECs. Adherent THP1 cells were quantified using ImageJ software. Bar graphs represent mean±SEM; *p<0.05, **p<0.01, n=3 (One-way ANOVA with Bonferroni's multiple comparison).

FIG. 7. Lipotoxic-EVs activate monocytes. Primary monocytes were treated with (A) EVs derived from the benign mouse hepatocyte cell line AML-12 for 4 hours and mRNA levels of TNF-α were assessed, or (B) EVs derived from primary mouse hepatocyte, and mRNA levels of MCP-1 (24 hours) and IL-1β (4 hours) were assessed by real-time PCR. Fold change was determined after normalization to Gapdh expression and expressed relative to that observed in control. Bar graphs represent mean±SEM; ***p<0.001,****p<0.0001; n=3 (Unpaired t test).

FIG. 8. Anti-ITGβ₁ antibody treatment does not alter neither the caloric intake nor the body or liver weight in FFC diet-fed mice. Wild type C57BL/6J mice were fed with chow or a diet high in saturated fat, fructose, and cholesterol (FFC) for 24 weeks, and 1 μg/g body weight of either anti-ITGβ₁ neutralizing antibody (ITGβ₁Ab) or control isotype antibody (IgG) was given twice per week for the last 4 weeks. (A) Daily caloric intake. (B) Body weight. (C) Liver weight at the time of sacrifice. ****p<0.0001; ns, nonsignificant, n=6 per group.

FIG. 9. Anti-ITGβ₁ antibody treatment did not alter neither the metabolic phenotype nor the steatosis in FFC diet-fed mice. Wild-type C57BL/6J mice were fed either chow or FFC diet for 24 weeks. 1 μg/g of either ITGβ₁Ab or control IgG isotype was given twice per week for the last 4 weeks. (A) Physical activity, energy expenditure, and respiratory quotient were assessed by CLAMS chambers. (B) Body weight curves. (C) Liver to body weight ratio at the time of sacrifice. (D) HOMA-IR at 23 weeks. (E) Hepatic triglyceride content. (F) Representative images of H&E staining of liver tissues (scale bar, 100 μm). Arrows indicate inflammatory cells infiltrate; ns, nonsignificant; n=5-6 per group.

FIG. 10. Anti-ITGβ₁ antibody treatment in FFC-fed mice attenuates hepatic inflammation. Wild type C57BL/6J mice were fed with chow or a diet high in saturated fat, fructose, and cholesterol (FFC) for 24 weeks, and 1 μg/g body weight of either anti-ITGβ₁ neutralizing antibody (ITGβ₁Ab) or control isotype antibody (IgG) was given twice per week for the last 4 weeks. (A) Representative images of immunohistochemistry for ITGβ₁ and quantification of ITGβ₁-positive areas. Scale bars: 100 μm. (B) Hepatic mRNA expression levels of Il12b, Il23α and Ccl2 were assessed by real-time PCR. Fold change was determined after normalization to 18 s expression in liver tissue and expressed relative to that observed in Chow-IgG mice; Bar graphs represent mean±SEM; n=5-6 per group ns: nonsignificant; *, ***, **** indicate statistical significance with p<0.05, p<0.001 and p<0.0001 respectively (One-way ANOVA with Bonferroni's multiple comparison).

FIG. 11. Anti-ITGβ₁ antibody treatment in FFC-fed mice attenuates hepatic inflammation. (A) Representative images of macrophage galactose-specific lectin (Mac-2) immunohistochemical staining of liver sections. (B) Mac-2 positive areas were quantified in 10 random 20× microscopic fields and averaged for each animal. (C) Hepatic mRNA expression levels of Cd68, Ccr2 and Tnf-α were assessed by real-time PCR. Fold change was determined after normalization to 18 s expression in liver tissue and expressed relative to that observed in Chow-IgG mice. (D) Flow cytometric analysis of the IHL population: top panels show the gating strategy; infiltrating monocytes were defined as CD45⁺CD11b^{hi}F4/80^{int} CCR2⁺cells. Bottom panels show quantification of each population. ns, nonsignificant; n=3-5 per group

FIG. 12. Intrahepatic leukocyte profiling by mass cytometry by time-of-flight (CyTOF). CyTOF was performed on IHL of chow-fed mice, and FFC-fed mice treated with either 1 μg/g of ITGβ₁Ab or control IgG isotype. IHL from IgG-treated chow-fed mice were used as control. (A) Twenty-eight unique clusters of IHL were defined by a 24 cell surface marker panel using the Rphenograph clustering algorhithm and were visualized on a t-distributed stochastic neighbor embedding (tSNE) plot using the R-based program Cytofkit. (B) Heat map demonstrating the distribution and relative intensity of the cell surface markers used in the clustering analysis. (C) Heat map showing the relative abundance of each cluster for each mouse. (D) Representative tSNE plots of each experimental group. The red color indicates high frequency categorization of cells to a cluster; the blue color indicates low frequency.

FIG. 13. Anti-ITGβ₁ antibody reduces the pro-inflammatory monocyte hepatic infiltration and increases the restorative macrophage population in the FFC-fed mice. Differentially expressed clusters between the groups (top graphs); clusters categorized into distinct leukocyte subpopulations based on intensities of individual cell surface markers (bottom graphs). (A) Cluster 5 and 9 represent infiltrating pro-inflammatory MoMF, (B) clusters 17, and 12 represent infiltrating MoMF, (C) cluster 1, 2 and 28 represent restorative macrophage, and (D) cluster 10 represents hepatic macrophage (n=3 per group).

FIG. 14. Anti-ITGβ₁ antibody treatment in FFC-fed mice does not alter the T lymphocyte, B lymphocyte, neutrophil and dendritic cells populations. Clusters were obtained by CyTOF, and categorized into distinct leukocyte subpopulations based on intensities of individual cell surface markers (bottom graphs of each cluster). Proportion of cells belonging to specific clusters were quantified for each experimental group (top graphs of each cluster; bar columns represent mean±SEM. *p<0.05, ns, nonsignificant; n=3 per group). (A) Cluster 8 was categorized into B cell-like cells. (B) Cluster 15 into neutrophil-like cells, (C) Cluster 19 into dendritic cell-like cells, and (D) cluster 16 and 21 into T-lymphocytes.

FIG. 15. Anti-ITGβ₁ antibody treatment reduces FFC diet-induced liver injury and fibrosis in murine NASH. (A) Representative images of TUNEL staining of liver sections, quantification of number of TUNEL-positive cells. (B) Serum ALT levels. (C) NAS scores. (D) Hepatic mRNA expression levels of Collagen1α1 and Osteopontin. (E) Representative images of Sirius red staining, quantification of Sirius red-positive areas. (F) Representative images of α-SMA staining of liver sections, quantification of α-SMA-positive areas. Scale bars: 100 μm; ns: nonsignificant; n=5-6 per group.

FIG. 16. Adhesion molecule VCAM-1 is upregulated in murine NASH. (A) The liver samples from chow-fed mice (n=3) and FFC-fed mice (n=3) were subjected to RNA-seq and the differential expression analyses were performed. Out of 13733 expressed coding transcripts, “candidate genes” gathered from previous literatures, known to encode adhesion molecules expressed on endothelial cells and mediate leukocyte adhesion are shown in the x-axis. The y-axis indicates log 2 fold change of mRNA abundance of each gene in FFC-fed mice liver relative to that in Chow-fed mice liver. The mRNA expression levels of Vcam1 and Icam1 in (B) whole liver lysates and (C) primary LSEC isolated from chow-fed mice and FFC-fed mice were evaluated by real-time qPCR. Fold change was determined after normalization to 18s mRNA expression, and expressed as fold change to that observed in chow-fed miceRepresentative images of VCAM-1 staining of liver sections from. (D) chow-fed mice (n=5) and FFC-fed mice (n=5) (left), (E) as well as human with normal liver, steatosis, and NASH with different stage of fibrosis. Scale bar: 100 μm. VCAM-1 positive areas were quantified in 10 random 10× microscopic fields and averaged for each animal or human subject. (F) TSEC were treated with vehicle (Veh) or 500 μM of PA for 16 hours. The mRNA expression levels of Vcam1 were evaluated by with the same methodology as that in (C). (H) Human primary LSEC were treated with vehicle or 250 μM of PA for 16 hours. The mRNA expression levels of Vcam1 were evaluated with the same methodology as that in (C). (G) LSEC isolated from wild-type C57BL/6J mice were treated with vehicle or 250 μM of PA for 16 hours. The mRNA expression levels of Vcam1 were evaluated with the same methodology as that in (C). (I) TSEC were treated with 50004 of PA for indicated periods of time. Protein levels of VCAM-1 were assessed by Western blot. GAPDH was used as a loading control. *p<0.05, **p<0.01, ***p<0.001, ****p<0.0001.

FIG. 17. VCAM-1 is upregulated in LSEC under lipotoxic conditions via MAPK pathway and NFκB dependent mechanism. (A) Schematic representation of MAPK pathway and exemplary MAPK pathway inhibitors. How lipotoxic stress modulate MAPK signaling cascade in LSEC has not been well studied so far. (B) TSEC were treated with 500 μM of PA for indicated periods of time. Protein levels of VCAM-1 and phosphorylated and total MKK3, p38, and JNK were assessed by Western blot. GAPDH was used as a loading control. (C) TSEC were treated with 500 μM of PA with or without indicated concentrations of MLK3 inhibitor URMC-099 (URMC) or p38 inhibitor SB203580 (SB) for 16 hours. The mRNA expression levels of Vcam1 were evaluated by real-time qPCR. Fold change was determined after normalization to 18s mRNA expression, and expressed as fold change to that observed in vehicle (Veh)-treated cells. (D) LSEC isolated from wild-type C57BL/6J mice were treated with 500 μM of PA with or without 2 μM of URMC-099. The mRNA expression levels of Vcam1 were evaluated with the same methodology as that in (C). *p<0.05, **p<0.01, ***p<0.001, ****p<0.0001.

FIG. 18. Anti-VCAM-1 antibody treatment alters neither the metabolic phenotype nor the steatosis in FFC diet-fed mice. Eight-week-old wild-type C57BL/6J mice were fed either chow or FFC diet for 24 weeks to induce NASH, and treated with either anti-VCAM-1 neutralizing antibody (VCAM1 Ab) or control IgG isotype antibody (IgG) twice a week for the last 4 weeks. (A) Body weight and Liver to body weight ratio at the time of sacrifice. (B) Daily caloric intake at 2 weeks after starting the antibody treatment. (C) Physical activity, respiratory quotient, and metabolic rate assessed by CLAMS study. (D) Representative images of H&E staining of liver tissues (scale bar, 100 μm). Arrows indicate inflammatory cells infiltrate. (E) NAS scores. (F) Hepatic triglyceride content. (G) HOMA-IR at one week before the sacrifice. n=5-7 per group; *p<0.05, **p<0.01, ***p<0.001, ****p<0.0001, ns, nonsignificant;

FIG. 19. Anti-VCAM-1 antibody treatment in FFC-fed mice attenuates hepatic injury and inflammation. (A) Representative images of TUNEL staining of liver sections (left). Scale bar: 100 μm. Quantification of TUNEL-positive cells (right). (B) Plasma ALT levels. (C) Representative images of macrophage galactose-specific lectin (Mac-2) staining of liver sections (left). Scale bar: 100 μm. Mac-2 positive areas were quantified in 10 random 20× microscopic fields and averaged for each animal (right). (D) Hepatic mRNA expression levels of Cd68, Ccr2, Tnfα, and Il1b were assessed by real-time PCR. Fold change was determined after normalization to 18s expression and expressed relative to Chow-IgG mice. n=5-7 per group; *p<0.05, **p<0.01, ***p<0.001, ****p<0.0001.

FIG. 20. Intrahepatic leukocyte profiling by mass cytometry by time-of-flight (CyTOF). CyTOF was performed on IHL of control IgG-treated chow-fed mice, and FFC-fed mice treated with either VCAM-1 neutralizing antibody or control IgG. (A) Thirty-one unique clusters of IHL were defined by a 30 cell surface marker panel (shown in Table 5) using the Rphenograph clustering algorithm and were visualized on a t-distributed stochastic neighbor embedding (tSNE) plot. (B) Representative tSNE plots of each group. Red dots indicate high frequency categorization of cells to a cluster; blue dots indicate low frequency. Red circles indicate clusters to which increased ratio of IHL were distributed in control IgG-treated FFC-fed mice and decreased ratio of IHL were distributed in VCAM1 Ab-treated FFC-fed mice. The blue circle indicate the cluster to which decreased ratio of IHL were distributed in control IgG-treated FFC-fed mice and increased ratio of IHL were distributed in VCAM1 Ab-treated FFC-fed mice. (C) Four clusters picked up in FIGS. 20A and 20B were categorized into distinct leukocyte subpopulations based on intensities of individual cell surface markers. n=3 per group

FIG. 21. Anti-VCAM-1 antibody treatment reduces FFC diet-induced liver fibrosis in murine NASH. (A) Representative images of Sirius red staining, quantification of Sirius red-positive areas. (B) Representative images of α-SMA staining of liver sections, quantification of α-SMA-positive areas. (C) Hepatic mRNA expressions of Collagen1α1 and Actα2 (α-SMA). Fold change was determined after normalization to 18s expression and expressed relative to Chow-IgG mice. Scale bar: 100 μm; n=5-7 per group; *p<0.05, **p<0.01, ***p<0.001, ****p<0.0001.

FIG. 22. Pathways related to leukocyte adhesion are profoundly activated in murine NASH liver. RNA-sequencing was performed on whole livers from FFC-fed NASH mice (n=3) and chow-fed control mice (n=3). Out of 13733 coding transcripts detected, 986 genes differentially upregulated in FFC-fed mice livers were subjected to Ingenuity Pathway Analysis (IPA). Top 10 ranked over-represented canonical pathways identified by IPA are shown. Filled circles indicate canonical pathways related to leukocyte adhesion and differentiation. Filled squares indicate canonical pathways in which Vcam1 is included.

FIG. 23. VCAM-1 is upregulated in LSEC in NASH via a mixed lineage kinase (MLK3) pathway. VCAM1 expression was assessed by immunohistochemistry in whole liver of chow and FFC-fed WT and mlk3^{-/-} mice, and quantified by morphometry.

FIG. 24. JNK inhibition did not alter VCAM1 expression in LSEC under lipotoxic conditions. TSEC were treated with either vehicle (Veh) or 500 μM of PA±SP600125 for 16 hours. (A) The mRNA expression levels of Vcam1 were evaluated by real-time qPCR. Fold change was determined after normalization to 18s mRNA expression, and expressed as fold change to that observed in vehicle-treated cells.

FIG. 25. B cells viability decrease during lipotoxic stress. Splenic B cells were isolated from C57BL/6J mouse treated with vehicle or 20 μM of LPC for 4 hours and cell viability was assessed. Relative numbers of viable cells were expressed as ratios (%) to those observed in vehicle-treated cells. Representative results of two independent experiments are shown. Bar graphs represents mean±SEM n=6 for each treatment group, ***p<0.001

FIG. 26. AGI-1067 inhibited PA-induced VCAM-1 upregulation in LSEC. TSEC were treated with either vehicle (Veh) or 500 μM of PA±AGI-1067 for 16 hours. (A) The mRNA expression levels of Vcam1 were evaluated by real-time qPCR. Fold change was determined after normalization to 18s mRNA expression, and expressed as fold change to that observed in vehicle-treated cells. (B) Protein level of VCAM-1 was assessed by Western blot. Beta-actin was used as a loading control.

FIG. 27. AGI-1067 treatment alters neither the metabolic phenotype nor the steatosis in FFC diet-fed mice. Wild-type C57BL/6J mice were fed either chow or FFC diet for 24 weeks to induce NASH, and treated with 25 mg/kg of AGI-1067 (AGI) or vehicle daily for the last 2 weeks of the feeding study. (A) Body weight and liver to body weight ratio at the time of sacrifice. (B) Daily caloric intake. (C) Representative images of H&E staining of liver tissues (scale bar, 100 μm). Arrows indicate inflammatory cells infiltrate. (D) NAS scores. (E) Hepatic triglyceride content. (F) HOMA-IR at 23 weeks of the feeding study e. n=4 for the Chow and Chow-AGI groups, n=5 for the FFC and FFC-AGI groups; *p<0.05, **p<0.01, ***p<0.001, ****p<0.0

FIG. 28. AGI-1067 treatment in FFC-fed mice attenuates hepatic injury, inflammation and fibrosis. Wild-type C57BL/6J mice were fed either chow or FFC diet for 24 weeks to induce NASH, and treated with or without 25 mg/kg of AGI-1067 (AGI) or vehicle daily for the last 2 weeks of the feeding study. (A) Plasma ALT levels. (B) Representative images of TUNEL staining of liver sections, quantification of TUNEL-positive cells. (C) Representative images of Galectin-3 staining of liver sections. Galectin-3 positive areas were quantified in 10 random 20× microscopic fields and averaged for each animal. (D) Hepatic mRNA expression levels of Cd68, Il1b, and TNFα were assessed by real-time PCR. Fold change was determined after normalization to 18s expression and expressed relative to chow-fed mice. (E) Representative images of Sirius red staining, quantification of Sirius red-positive areas. (F) Representative images of α-SMA staining of liver sections, quantification of α-SMA-positive areas. (G) Hepatic mRNA expressions of Collagen1α1 and Acta2 (α-SMA) assessed by real-time PCR. Fold change was determined after normalization to 18s expression and expressed relative to chow-fed mice. Scale bars: 100 μm; n=4 for the Chow-AGI group, n=4 for the Chow and Chow-AGI groups, n=5 for the FFC and FFC-AGI groups; *p<0.05, **p<0.01, ***p<0.001, ****p<0.0001, ns, nonsignificant.

### DETAILED DESCRIPTION

This document provides methods and materials involved in treating chronic liver disease (e.g., non-alcoholic fatty liver disease such as NASH). For example, this document provides methods and materials for administering one or more inhibitors of an ITGβ1 polypeptide, one or more inhibitors of an ITGα9 polypeptide, and/or one or more inhibitors of a VCAM-1 polypeptide to a mammal (e.g., a human) having a chronic liver disease to treat chronic liver disease.

Any appropriate mammal having chronic liver disease (e.g., non-alcoholic fatty liver disease such as NASH) can be treated as described herein (e.g., by administering one or more inhibitors of an ITGβ1 polypeptide, one or more inhibitors of an ITGα9 polypeptide, and/or one or more inhibitors of a VCAM-1 polypeptide). In some cases, a mammal can be an obese mammal. In some cases, a mammal can have type 2 diabetes. In some cases, a mammal can have a metabolic syndrome. Examples of mammals that can be treated as described herein include, without limitation, humans, non-human primates such as monkeys, dogs, cats, horses, cows, pigs, sheep, mice, and rats.

When treating a mammal (e.g., a human) having a chronic liver disease as described herein (e.g., by administering one or more inhibitors of an ITGβ1 polypeptide, one or more inhibitors of an ITGα9 polypeptide, and/or one or more inhibitors of a VCAM-1 polypeptide), the chronic liver disease can be any type of chronic liver disease. In some cases, a chronic liver disease to be treated as described herein can be a fatty liver disease such as a non-alcoholic fatty liver disease. Examples of chronic liver diseases that can be treated as described herein include, without limitation, NASH, and non-alcoholic fatty liver (NAFL; simple fatty liver). In cases where a chronic liver disease to be treated as described herein is non-alcoholic fatty liver disease, the non-alcoholic fatty liver disease can be a diet-induced non-alcoholic fatty liver disease such as diet-induced NASH.

In some cases, a mammal (e.g., a human) can be identified as having a chronic liver disease (e.g., non-alcoholic fatty liver disease such as NASH). Any appropriate method can be used to identify a mammal as having a chronic liver disease. For example, blood tests (e.g., complete blood counts, liver enzyme and liver function tests, tests for chronic viral hepatitis, celiac disease screening tests, fasting blood sugar tests, hemoglobin A1C tests, and lipid profile tests), imaging procedures (e.g., ultrasound, computerized tomography (CT) scanning, or magnetic resonance imaging (MRI), transient elastography, and magnetic resonance elastography), and/or liver tissue examination (e.g., to look for signs of inflammation and scarring) can be used to identify a mammal as having a chronic liver disease such as a fatty liver disease.

Once identified as having a chronic liver disease, a mammal can be administered one or more inhibitors of an ITGβ1 polypeptide, one or more inhibitors of an ITGα9 polypeptide, and/or one or more inhibitors of a VCAM-1 polypeptide.

In some cases, one or more inhibitors of an ITGβ1 polypeptide, one or more inhibitors of an ITGα9 polypeptide, and/or one or more inhibitors of a VCAM-1 polypeptide can be administered to a mammal having a chronic liver disease to reduce or eliminate one or more symptoms of chronic liver disease. Examples of symptoms of chronic liver disease that can be reduced or eliminated as described herein include, without limitation, enlarged liver, fatigue, pain in the upper right abdomen, abdominal swelling (ascites), enlarged blood vessels just beneath the skin's surface, enlarged breasts in men, enlarged spleen, red palms, and yellowing of the skin and eyes (jaundice).

In some cases, one or more inhibitors of an ITGβ1 polypeptide, one or more inhibitors of an ITGα9 polypeptide, and/or one or more inhibitors of a VCAM-1 polypeptide can be administered to a mammal having a chronic liver disease to reduce or eliminate liver inflammation in the mammal. Any appropriate method can be used to determine whether or not liver tissue has a reduced or eliminated level of inflammation. For example, a reduced level of monocyte-derived macrophages (MoMFs), a reduced level of one or more pro-inflammatory polypeptides (e.g., Cd68 polypeptides and Tnf-α polypeptides), and/or an increased level of one or more anti-inflammatory polypeptides (e.g., CD206 polypeptides, Lgals polypeptides, MERTK polypeptides, and F4/80 polypeptides) can be used to determine whether or not liver tissue contains a reduced or eliminated level of inflammation. A reduced level of MoMFs and/or a pro-inflammatory polypeptide can be any level that is lower than a level of that polypeptide that was observed prior to administration of one or more inhibitors of an ITGβ1 polypeptide, one or more inhibitors of an ITGα9 polypeptide, and/or one or more inhibitors of a VCAM-1 polypeptide. An increased level of an anti-inflammatory polypeptide can be any level that is higher than a level of that polypeptide that was observed prior to administration of one or more inhibitors of an ITGβ1 polypeptide, one or more inhibitors of an ITGα9 polypeptide, and/or one or more inhibitors of a VCAM-1 polypeptide. For example, one or more inhibitors of an ITGβ1 polypeptide, one or more inhibitors of an ITGα9 polypeptide, and/or one or more inhibitors of a VCAM-1 polypeptide can be effective to reduce inflammation in liver tissue (e.g., liver tissue in a mammal such as a human) by, for example, 10, 20, 30, 40, 50, 60, 70, 80, 90, 95, or more percent.

In some cases, one or more inhibitors of an ITGβ1 polypeptide, one or more inhibitors of an ITGα9 polypeptide, and/or one or more inhibitors of a VCAM-1 polypeptide can be administered to a mammal having a chronic liver disease to reduce or eliminate liver fibrosis in the mammal. Any appropriate method can be used to determine whether or not liver tissue has a reduced or eliminated level of fibrosis. For example, reduced levels of one or more polypeptides involved in fibrosis (e.g., collagen 1a1 polypeptides and osteopontin polypeptides) can be used to determine whether or not liver tissue contains a reduced or eliminated level of fibrosis. A reduced level of a polypeptide involved in fibrosis can be any levels of a polypeptide involved in fibrosis that is lower than a level of that polypeptide that was observed prior to administration of one or more inhibitors of an ITGβ1 polypeptide, one or more inhibitors of an ITGα9 polypeptide, and/or one or more inhibitors of a VCAM-1 polypeptide. For example, one or more inhibitors of an ITGβ1 polypeptide, one or more inhibitors of an ITGα9 polypeptide, and/or one or more inhibitors of a VCAM-1 polypeptide can be effective to reduce fibrosis in liver tissue (e.g., liver tissue in a mammal such as a human) by, for example, 10, 20, 30, 40, 50, 60, 70, 80, 90, 95, or more percent.

An inhibitor of an ITGβ1 polypeptide can be any appropriate inhibitor of an ITGβ1 polypeptide. An inhibitor of an ITGβ1 polypeptide can be an inhibitor of ITGβ1 polypeptide activity or an inhibitor of ITGβ1 polypeptide expression. In some cases, an inhibitor of an ITGβ1 polypeptide can cause an integrin heterodimer including an ITGα9β1 heterodimer to adopt a closed conformation (e.g., a formation that has a low affinity for ligand). Examples of compounds that can reduce ITGβ1 polypeptide activity include, without limitation, antibodies (e.g., neutralizing antibodies) and small molecules (e.g., a pharmaceutically acceptable salt of a small molecule). Examples of neutralizing anti-ITGβ1 polypeptide antibodies that can be used as described herein include, without limitation, those antibodies listed in Table 1. Examples of small molecule inhibitors of ITGβ1 polypeptide activity include, without limitation, BOP (R&D Cat. No. 6047), and R-BC154 (R&D Cat. No. 6048).

TABLE 1
Exemplary neutralizing anti- ITGβ1 polypeptide antibodies.

| Antibody Name | Source/Supplier/Reference |
|---|---|
| Ab52971 | Sigma |
| MAB13 | BD Pharmingen 552828 |
| 9EG7 | BD Pharmingen 553715 |
| Hmb1-1 | eBioscience |

Examples of compounds that can reduce ITGβ1 polypeptide expression and be used as described herein include, without limitation, nucleic acid molecules designed to induce RNA interference against ITGβ1 polypeptide expression (e.g., a siRNA molecule or a shRNA molecule), antisense molecules against ITGβ1 polypeptide expression, and miRNAs against ITGβ1 polypeptide expression. Examples of nucleic acid molecules designed to induce RNA interference against ITGβ1 polypeptide expression that can be used as described herein include, without limitation, shRNA including the nucleic acid sequence CCGGGCCTTGCA-TTACTGCTGATATCTCGAGATATCAGCAGTAATGCAAGGCTTTTTG (SEQ ID NO:1). In some cases, an inhibitor of an ITGβ1 polypeptide can reduce or prevent formation of an integrin heterodimer including ITGβ1 and an ITG α subunit (e.g., ITGα9). In some cases, an inhibitor of an ITGβ1 polypeptide can reduce or eliminated binding (e.g., covalently binding) between an ITGα9β1 heterodimer and an ITGα9β1 heterodimer binding partner (e.g., an ITGα9β1 heterodimer ligand such as VCAM-1).

An inhibitor of an ITGα9 polypeptide can be any appropriate inhibitor of an ITGα9 polypeptide. An inhibitor of an ITGα9 polypeptide can be an inhibitor of ITGα9 polypeptide activity or an inhibitor of ITGα9 polypeptide expression. In some cases, an inhibitor of an ITGα9 polypeptide can cause an ITGα9β1 heterodimer to adopt a closed conformation (e.g., a formation that has a low affinity for ligand). Examples of compounds that can reduce ITGα9 polypeptide activity include, without limitation, antibodies (e.g., neutralizing antibodies) and small molecules (e.g., a pharmaceutically acceptable salt of a small molecule). Examples of neutralizing anti-ITGα9 polypeptide antibodies that can be used as described herein include, without limitation, those antibodies listed in Table 2. Examples of small molecule inhibitors of ITGα9 polypeptide activity include, without limitation, BOP (R&D Cat. No. 6047), and R-BC154 (R&D Cat. No. 6048).

TABLE 2
Exemplary neutralizing anti-ITGα9 polypeptide antibodies.

| Antibody Name | Source/Supplier/Reference |
|---|---|
| MAB4574 | R&D |

Examples of compounds that can reduce ITGα9 polypeptide expression and be used as described herein include, without limitation, nucleic acid molecules designed to induce RNA interference against ITGα9 polypeptide expression (e.g., a siRNA molecule or a shRNA molecule), antisense molecules against ITGα9 polypeptide expression, and miRNAs against ITGα9 polypeptide expression. In some cases, an inhibitor of an ITGα9 polypeptide can reduce or prevent formation of an ITGα9β1 heterodimer. In some cases, an inhibitor of an ITGα9 polypeptide can reduce or eliminated binding (e.g., covalently binding) between an ITGα9β1 heterodimer and an ITGα9β1 heterodimer binding partner (e.g., an ITGα9β1 heterodimer ligand such as VCAM-1).

An inhibitor of a VCAM-1 polypeptide can be any appropriate inhibitor of a VCAM-1 polypeptide. An inhibitor of a VCAM-1 polypeptide can be an inhibitor of VCAM-1 polypeptide activity or an inhibitor of VCAM-1 polypeptide expression. Examples of compounds that can reduce VCAM-1 polypeptide activity include, without limitation, antibodies (e.g., neutralizing antibodies) and small molecules (e.g., a pharmaceutically acceptable salt of a small molecule). Examples of neutralizing anti-VCAM-1 polypeptide antibodies that can be used as described herein include, without limitation, those antibodies listed in Table 3, those antibodies described in U.S. Pat. No. 7,449,186, those antibodies described in WO 2013/160676, and those antibodies described in WO 2013/026878. Examples of small molecule inhibitors of VCAM-1 polypeptide activity include, without limitation, AGI-1067, BAY 11-7082, IkappaBalpha kinase inhibitor, and a delta-tocotrienol form of vitamin E.

TABLE 3
Exemplary neutralizing anti-VCAM-1 polypeptide antibodies.

| Antibody Name | Source/Supplier/Reference |
|---|---|
| BBA5 | R&D |
| AF809 | R&D |
| AF643 | R&D |
| 6G10 | hybridoma ATTC No. HB 10519 |

Examples of compounds that can reduce VCAM-1 polypeptide expression and be used as described herein include, without limitation, nucleic acid molecules designed to induce RNA interference against VCAM-1 polypeptide expression (e.g., a siRNA molecule or a shRNA molecule), antisense molecules against VCAM-1 polypeptide expression, and miRNAs against VCAM-1 polypeptide expression. In some cases, an inhibitor of a VCAM-1 polypeptide can reduce or eliminated binding (e.g., covalently binding) between a VCAM-1 polypeptide and a VCAM-1 polypeptide binding partner (e.g., an ITGβ1 polypeptide).

One or more inhibitors of an ITGβ1 polypeptide, one or more inhibitors of an ITGα9 polypeptide, and/or one or more inhibitors of a VCAM-1 polypeptide can be administered to a mammal having a chronic liver disease (e.g., non-alcoholic fatty liver disease such as NASH) at the same time or independently. When one or more inhibitors of an ITGβ1 polypeptide, one or more inhibitors of an ITGα9 polypeptide, and/or one or more inhibitors of a VCAM-1 polypeptide are administered at the same time, one or more inhibitors of an ITGβ1 polypeptide, one or more inhibitors of an ITGα9 polypeptide, and/or one or more inhibitors of a VCAM-1 polypeptide can be administered as separate compositions administered at the same time or can be present in a single composition.

In some cases, one or more inhibitors of an ITGβ1 polypeptide, one or more inhibitors of an ITGα9 polypeptide, and/or one or more inhibitors of a VCAM-1 polypeptide can be administered to a mammal having a chronic liver disease (e.g., non-alcoholic fatty liver disease such as NASH) as the sole active ingredient(s) used to treat the chronic liver disease. For example, in some cases, when using a composition containing an inhibitor of an ITGβ1 polypeptide, that inhibitor of an ITGβ1 polypeptide can be the sole active ingredient that treats the chronic liver disease.

In some cases, one or more inhibitors of an ITGβ1 polypeptide, one or more inhibitors of an ITGα9 polypeptide, and/or one or more inhibitors of a VCAM-1 polypeptide can be administered to a mammal having a chronic liver disease (e.g., non-alcoholic fatty liver disease such as NASH) as a combination therapy with one or more additional treatments used to treat a chronic liver disease. For example, a combination therapy used to treat chronic liver disease can include administering to the mammal (e.g., a human) one or more inhibitors of an ITGβ1 polypeptide, one or more inhibitors of an ITGα9 polypeptide, and/or one or more inhibitors of a VCAM-1 polypeptide as described herein and one or more chronic liver disease treatments such as anti-inflammatory drugs (e.g., pentoxifylline such as Trental® and obeticholic acid such as Ocaliva®), antioxidants (e.g., vitamin E), thiazolidinediones (e.g., pioglitazone), and/or obeticholic acid. In cases where one or more inhibitors of an ITGβ1 polypeptide, one or more inhibitors of an ITGα9 polypeptide, and/or one or more inhibitors of a VCAM-1 polypeptide described herein are used in combination with one or more additional chronic liver disease treatments, the one or more additional chronic liver disease treatments can be administered at the same time or independently. For example, one or more inhibitors of an ITGβ1 polypeptide, one or more inhibitors of an ITGα9 polypeptide, and/or one or more inhibitors of a VCAM-1 polypeptide described herein can be administered first, and the one or more additional chronic liver disease treatments can be administered second, or vice versa.

In some cases, one or more inhibitors of an ITGβ1 polypeptide, one or more inhibitors of an ITGα9 polypeptide, and/or one or more inhibitors of a VCAM-1 polypeptide can be formulated into a composition (e.g., pharmaceutically acceptable composition) for administration to a mammal having a chronic liver disease (e.g., non-alcoholic fatty liver disease such as NASH). For example, a therapeutically effective amount of one or more inhibitors of an ITGβ1 polypeptide, one or more inhibitors of an ITGα9 polypeptide, and/or one or more inhibitors of a VCAM-1 polypeptide can be formulated together with one or more pharmaceutically acceptable carriers (additives) and/or diluents. A pharmaceutical composition can be formulated for administration in any appropriate dosage form. Examples of dosage forms that can be used as described herein include, without limitation, solid and liquid forms such as gums, capsules, tablets (e.g., chewable tablets, and enteric coated tablets), suppository, liquid, enemas, suspensions, solutions (e.g., sterile solutions), sustained-release formulations, delayed-release formulations, pills, powders, gels, creams, ointments, and granules. Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, ascorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol such as Vitamin E TPGS, sodium carboxymethylcellulose, polyacrylates, waxes, polyethylene-polyoxypropylene-block polymers, and wool fat.

A composition (e.g., a pharmaceutical composition) containing one or more inhibitors of an ITGβ1 polypeptide, one or more inhibitors of an ITGα9 polypeptide, and/or one or more inhibitors of a VCAM-1 polypeptide can be designed for oral or parenteral (including subcutaneous, intramuscular, intratumoral, intravenous, topical, and intradermal) administration. When being administered orally, a pharmaceutical composition containing one or more inhibitors of an ITGβ1 polypeptide, one or more inhibitors of an ITGα9 polypeptide, and/or one or more inhibitors of a VCAM-1 polypeptide can be in the form of a pill, syrup, gel, liquid, flavored drink, tablet, or capsule. Compositions suitable for parenteral administration include, without limitation, aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

A composition (e.g., a pharmaceutical composition) containing one or more inhibitors of an ITGβ1 polypeptide, one or more inhibitors of an ITGα9 polypeptide, and/or one or more inhibitors of a VCAM-1 polypeptide can be administered locally or systemically. For example, a composition containing one or more inhibitors of an ITGβ1 polypeptide, one or more inhibitors of an ITGα9 polypeptide, and/or one or more inhibitors of a VCAM-1 polypeptide can be administered systemically by an oral administration or by injection to a mammal (e.g., a human).

Effective doses of one or more inhibitors of an ITGβ1 polypeptide, one or more inhibitors of an ITGα9 polypeptide, and/or one or more inhibitors of a VCAM-1 polypeptide can vary depending on the severity of the chronic liver disease (e.g., non-alcoholic fatty liver disease such as NASH), the route of administration, the age and general health condition of the subject, excipient usage, the possibility of co-usage with other therapeutic treatments such as use of other agents, the specific inhibitor being used, and/or the judgment of the treating physician.

An effective amount of a composition containing one or more inhibitors of an ITGβ1 polypeptide, one or more inhibitors of an ITGα9 polypeptide, and/or one or more inhibitors of a VCAM-1 polypeptide can be any amount that can treat the chronic liver disease (e.g., non-alcoholic fatty liver disease such as NASH) without producing significant toxicity to the mammal. An effective amount of an inhibitor of an inhibitor of an ITGβ1 polypeptide, an inhibitor of an ITGα9 polypeptide, and/or an inhibitor of a VCAM-1 polypeptide can be any appropriate amount. In some cases, an effective amount of an inhibitor of an ITGβ1 polypeptide such as a neutralizing antibody (e.g., an ITGβ1 neutralizing antibody) can be from about 1 mg/kg body weight of a mammal to about 10 mg/kg body weight of a mammal (e.g., from about 1 mg/kg body to about 8 mg/kg, from about 1 mg/kg body to about 5 mg/kg, from about 1 mg/kg body to about 4 mg/kg, from about 1 mg/kg body to about 3 mg/kg, from about 1 mg/kg body to about 2 mg/kg, from about 2 mg/kg body to about 10 mg/kg, from about 3 mg/kg body to about 10 mg/kg, from about 5 mg/kg body to about 10 mg/kg, from about 7 mg/kg body to about 10 mg/kg, from about 2 mg/kg body to about 8 mg/kg, from about 3 mg/kg body to about 5 mg/kg, or about 1 mg/kg body weight of a mammal). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the chronic liver disease (e.g., non-alcoholic fatty liver disease such as NASH) may require an increase or decrease in the actual effective amount administered.

The frequency of administration of a composition containing one or more inhibitors of an ITGβ1 polypeptide, one or more inhibitors of an ITGα9 polypeptide, and/or one or more inhibitors of a VCAM-1 polypeptide can be any frequency that can treat the chronic liver disease (e.g., non-alcoholic fatty liver disease such as NASH) without producing significant toxicity to the mammal. For example, the frequency of administration can be from about three times a day to about once a week, from about twice a day to about twice a week, or from about once a day to about twice a week. The frequency of administration can remain constant or can be variable during the duration of treatment. A course of treatment with a composition containing one or more inhibitors of an ITGβ1 polypeptide, one or more inhibitors of an ITGα9 polypeptide, and/or one or more inhibitors of a VCAM-1 polypeptide can include rest periods. For example, a composition containing one or more inhibitors of an ITGβ1 polypeptide, one or more inhibitors of an ITGα9 polypeptide, and/or one or more inhibitors of a VCAM-1 polypeptide can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and/or severity of the chronic liver disease (e.g., non-alcoholic fatty liver disease such as NASH) may require an increase or decrease in administration frequency.

An effective duration for administering a composition containing one or more inhibitors of an ITGβ1 polypeptide, one or more inhibitors of an ITGα9 polypeptide, and/or one or more inhibitors of a VCAM-1 polypeptide can be any duration that treat the chronic liver disease (e.g., non-alcoholic fatty liver disease such as NASH) without producing significant toxicity to the mammal. For example, the effective duration can vary from several days to several weeks, months, or years. In some cases, the effective duration for the treatment of chronic liver disease can range in duration from about one month to about 10 years. Multiple factors can influence the actual effective duration used for a particular treatment. For example, an effective duration can vary with the frequency of administration, effective amount, use of multiple treatment agents, route of administration, and/or severity of the condition being treated.

In some cases, the materials and methods described herein also can be used to treat other diseases or disorders that are characterized by aberrant signaling from an integrin receptor including α9 and β1 subunits interacting with a VCAM-1 ligand.

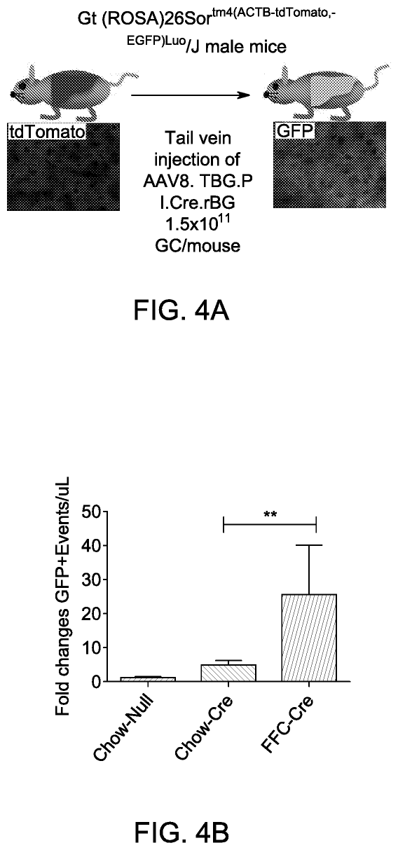

This document also provides non-human animal models (e.g., a mouse model) of chronic liver disease (e.g., non-alcoholic fatty liver disease such as NASH). As described herein, ITGα9β1 heterodimers are released from hepatocytes under lipotoxic stress as a cargo of EVs (also see, e.g., Ibrahim et al., Hepatology. 63(3):731-44 (2016); and Hirsova et al., Gastroenterology, 150(4):956-67 (2016)). Non-human animal models for chronic liver disease described herein can be non-human animals in which circulating EVs of hepatocyte origin can be tracked. In some cases, a non-human animal model for chronic liver disease described herein (e.g., in which circulating EVs of hepatocyte origin can be tracked) can be generated using a non-human animal engineered to have a cell membrane-targeted, dual (e.g., a two-color fluorescent) Cre-reporter allele and a Cre recombinase. A cell membrane-targeted, dual reporter allele can be used to detect a cellular membranous structures such as the cytoplasmic membrane, and can allow labelling of membrane-derived structures such as exosomes and EVs. In the absence of a Cre recombinase (e.g., prior to any Cre recombination), a non-human animal engineered to have a cell membrane-targeted, dual (e.g., a two-color fluorescent) Cre-reporter allele expresses a first cell membrane-localized label (e.g., a first fluorescent label), and in the presence of a Cre recombinase (e.g., following Cre recombination), a non-human animal engineered to have a cell membrane-targeted, dual (e.g., a two-color fluorescent) Cre-reporter allele can switch to expressing a second cell membrane-localized label (e.g., a second fluorescent label). For example, a non-human animal engineered to have a cell membrane-targeted, two-color fluorescent Cre-reporter allele can include, prior to any Cre recombination, a cell membrane-localized first fluorescent label (e.g., a red fluorescent protein such as tdTomato), and can include, following Cre recombination, a cell membrane-localized second fluorescent label (e.g., a green fluorescent protein (GFP) such as enhanced GFP (EGFP)). In some cases, a non-human animal model for chronic liver disease in which circulating EVs of hepatocyte origin can be tracked can be as described in Example 1. In some cases, a non-human animal model for chronic liver disease in which circulating EVs of hepatocyte origin can be tracked can be as shown in FIG. 4A.

Any appropriate non-human animal engineered to have a cell membrane-targeted, dual (e.g., a two-color fluorescent) Cre-reporter allele can be used to generate a non-human animal model for chronic liver disease described herein (e.g., a non-human animal model having labelled hepatocyte-derived EVs). In some cases, a non-human animal engineered to have a cell membrane-targeted, dual (e.g., a two-color fluorescent) Cre-reporter allele can be as described in Example 1. In some cases, a non-human animal engineered to have a cell membrane-targeted, dual (e.g., a two-color fluorescent) Cre-reporter allele can be as described elsewhere (see, e.g., The Jackson Laboratory Strain No. 007676; The Jackson Laboratory Strain No. 007576; and Muzumdar et al., Genesis 45(9):593-605 (2007)).

Any appropriate Cre recombinase can be used to generate a non-human animal model for chronic liver disease described herein (e.g., a non-human animal model having labelled hepatocyte-derived EVs). A Cre recombinase can be provided using any appropriate method. In some cases, a Cre recombinase can be provided by administering a Cre recombinase to a non-human animal engineered to have a cell membrane-targeted, two-color fluorescent Cre-reporter allele. In some cases, a Cre recombinase can be provided by administering a nucleic acid encoding a Cre recombinase to a non-human animal engineered to have a cell membrane-targeted, two-color fluorescent Cre-reporter allele. In cases where a nucleic acid encoding a Cre recombinase is administered to a non-human animal engineered to have a cell membrane-targeted, dual Cre-reporter allele, the nucleic acid can be any appropriate type of nucleic acid. For example, an expression vector or a viral vector such as an adeno-associate viral (AAV) vector (e.g., AAV8) encoding a Cre recombinase can be used to administer a Cre recombinase to a non-human animal engineered to have a cell membrane-targeted, dual Cre-reporter allele. In cases where a nucleic acid encoding a Cre recombinase is a viral vector, the viral vector can be derived from a virus having hepatotropism (e.g., AAV8). To generate labelled (e.g., fluorescently labelled) EVs of hepatocyte origin, Cre recombination can be provided in hepatocytes (e.g., but not in other cell types). For example, in cases where a nucleic acid encoding a Cre recombinase is administered to a non-human animal engineered to have a cell membrane-targeted, dual Cre-reporter allele, the nucleic acid encoding a Cre recombinase can be under the control of a hepatocyte specific promoter. Examples of hepatocyte specific promoters include, without limitation, a thyroxine binding globulin (TBG) promoter.

A non-human animal model for chronic liver disease can be a model for any appropriate type of chronic liver disease (e.g., non-alcoholic fatty liver disease such as NASH). In some cases, a non-human animal model for chronic liver disease can be a model for diet-induced NASH. In some cases, a non-human animal model for chronic liver disease can exhibit one or more symptoms of chronic liver disease. Examples of symptoms of chronic liver disease include, without limitation, enlarged liver, fatigue, pain in the upper right abdomen, abdominal swelling (ascites), enlarged blood vessels just beneath the skin's surface, enlarged breasts in males, enlarged spleen, red palms, and yellowing of the skin and eyes (jaundice).

The invention will be further described in the following examples, which do not limit the scope of the invention described in the claims.

### EXAMPLES

### Example 1: Integrin β₁-Enriched Extracellular Vesicles Mediate Monocyte Adhesion and Promote Liver Inflammation in Murine NASH

Hepatic recruitment of monocyte-derived macrophages (MoMF) contributes to the inflammatory response in nonalcoholic steatohepatitis (NASH). However, how hepatocyte lipotoxicity promotes MoMF inflammation is unclear. This Example demonstrates that lipotoxic hepatocyte-derived extracellular vesicles (EVs) are enriched with active integrin β₁ (ITGβ₁), which promotes monocyte adhesion and liver inflammation in murine NASH.

### Materials and Methods

### Materials

LPC (Sigma, St. Louis, Mo.) was dissolved as described elsewhere (see, e.g., Kakisaka et al., Am J Physiol Gastrointest Liver Physiol, 302(1):G77-84 (2012)). Primary antisera employed for the studies include: anti-integrin beta 1 (ITGβ₁) (Ab52971), anti-ITGα₅ (Ab150361), anti-ITGα_{V} (Ab179475), anti-ITGα₄ (ab81280), anti-TSG101 (Ab125011), and anti-alpha smooth muscle actin (α-SMA) (ab5694 or ab124964) antibodies from Abcam (Cambridge, Mass.), anti-ITGα₉ (MAB4574) from R&D (Minneapolis, Minn.), anti-talin-1 (4021), anti-Rab7 (9367), anti-LAMP1 (9091) from Cell Signaling Technology (Danvers, Mass.), anti-GAPDH (MAB374) from Millipore Sigma (St. Louis, Mo., USA), anti-β-actin (sc-47778) from Santa Cruz Biotechnologies (Santa Cruz, Calif.), anti-ITGβ₁ MAB13 (552828), anti-ITGβ₁ 9EG7 (553715), and isotype IgG control antibody (553927), anti-EEA1 (610456) from BD Pharmingen, (San Diego, Calif.), and anti-CD63 (MA1-19602), anti-Mac-2 (14530181) from Thermo Fisher Scientific (Waltham, Mass.), anti-ITGα9 antibody (MAB2078Z) from Millipore Sigma (St. Louis, Mo., USA), and anti-VCAM1 antibody (BBAS) from R&D (Minneapolis, Minn.).

### Cells

The human hepatoma cell line Huh7 originating from a well differentiated hepatocellular carcinoma in a 57-year-old male patient, the murine hepatocyte cell line AML12 derived from a male mouse transgenic for transforming growth factor-α, and the human monocyte cell line THP1 derived from a 1-year-old boy with acute monocytic leukemia were purchased form ATCC (Rockville, Md.). Huh7 cells were cultured in Dulbecco's modified Eagle's medium (DMEM) supplemented with 10% fetal bovine serum, and primocin (100 mg/ml) (InvivoGen, San Diego, Calif.). AML12 cells were cultured in DMEM/F12 medium supplemented with 10% fetal bovine serum, 1% Insulin-Transferrin-Selenium (ITS; Gibco, Grand Island, N.Y.), dexamethasone (40 ng/mL), and primocin (100 mg/ml). Primary mouse hepatocytes (PMHs) were isolated by collagenase perfusion and purified by Percoll (Sigma-Aldrich, St. Louis, Mo.) gradient centrifugation as described elsewhere (see, e.g., Hirsova et al., PLoS One, 8(7):e70599 (2013)). THP1 cells were cultured in RPMI-1640 medium supplemented with 10% fetal bovine serum. Mouse primary monocytes were isolated from mouse bone marrow using EasySep mouse monocyte isolation kit (StemCell Technologies, Vancouver, BC, Canada) and cultured in RPMI-1640 medium supplemented with 10% fetal bovine serum and primocin (100 mg/ml). The human liver sinusoidal endothelial cells (LSECs) were purchased from ScienCell Research Laboratories (San Diego, Calif.), and cultured in Endothelial Cell Growth Medium (ECM, ScienCell Research Laboratories) consisting of 5% FBS, 1% endothelial cells growth supplement, and 1% penicillin/streptomycin solution. Primary mouse LSECs were isolated from collagenase perfused liver using CD146 (LSEC) MicroBeads (Miltenyi Biotec, Bergisch Gladbach, Germany) following the manufacture's instruction and cultured in the above Endothelial Cell Growth Medium. All the cell cultures were maintained at 37° C. in a humidified atmosphere of 5% CO2.

ITGβ₁ shRNA Knockdown Cell Line

TRC2 pLKO.5-Puro ITGβ₁ shRNA-CCGGGCCTTGCATTACTGCTGATATCTCGAGATATCAGCAGTAATGCAAGGCT TTTTG (SEQ ID NO:1) was introduced to pack lentivirus by co-transfecting HEK293T cells with packaging vectors psPAX2 (viral proteins Gag and Rev under the SV40 promoter; Addgene plasmid #12260, a gift from D. Trono, École Polytechnique Federate de Lausanne, Lausanne, Switzerland) and pMD2.G (viral protein VSV-G expressed under the CMV promoter; Addgene plasmid #12259). Virus was harvested at 48 and 72 hours after transfection and filtered with a 0.45 μm filter. Huh7 cells were transduced by incubating with viral supernatant (diluted 1:2) in the presence of 8 μg/mL polybrene (Sigma) for 24 hours. Infected cells were selected by treating with 2 μg/mL puromycin (InvivoGen). The efficiency of shRNA mediated gene knockdown was examined by immunoblot analysis.

### Extracellular Vesicles Isolation, Labeling and Quantification

EVs were isolated from cell culture medium by differential ultracentrifugation as described elsewhere (see, e.g., Ibrahim et al., Hepatology, 63(3):731-44 (2016); and Hirsova et al., Gastroenterology, 150(4):956-67 (2016)). Collected medium was depleted of cells and cell debris initially by low-speed centrifugations (2,000 g for 20 minutes and 20,000 g, for 30 minutes). The supernatants were collected and centrifuged for 90 minutes at 100,000 g at 4° C. For labeling EVs, DiO dye (Thermo Fisher) was added to the pellet, which was re-suspended in PBS and ultracentrifuged for 90 minutes at 100,000 g at 4° C., twice. For functional and characterization studies pellets from the first ultracentrifugation step were washed in PBS, and centrifuged again for 90 minutes at 100,000 g at 4° C. The obtained pellets were lysed in lysis buffer, or re-suspended in PBS solution or RPMI-1640 medium, depending on the subsequent experiments. EVs used for monocyte treatment were sterile filtered through 0.22 μm syringe filter. For the functional assay, EVs were isolated using ultrafiltration and size-exclusion chromatography (UF-SEC). Briefly, conditioned media were harvested and centrifuged for 15 minutes at 5,000 g to remove cells and cell debris. Next, media were filtered through a 0.22 μm filter. For UF-SEC, media were loaded onto Amicon Ultra-15 Centrifugal Filter Units with Ultracel-10 membrane (MWCO=10 kDa; Millipore, Billerica, Mass.) and concentrated by repeated centrifugation at 4000×g. After sample volume was adjusted to 500 μl, the concentrate media was applied to a 10 ml sepharose CL-4B column (GE Healthcare) and 20 fractions of 0.5 ml were collected using PBS as an eluent. The EV rich fractions were collected from fractions 5-12 then concentrated to 250 μl on an Amicon Ultra-4 Centrifugal Filter Unit with Ultracel-10 membrane (MWCO=10 kDa; Millipore) by centrifugation at 4,000 g and stored at 80° C. Concentration and size distribution of isolated EVs were assessed by nanoparticle-tracking analysis (NTA) using NanoSight NS300 instrument (NanoSight Ltd., Amesbury, UK). Briefly, EV samples were diluted with PBS. Each sample was continuously run through a flow-cell top-plate using a syringe pump at a rate of 25 μL/minute. At least three videos of 30 seconds documenting Brownian motion of nanoparticles were recorded, and at least 1000 completed tracks were analyzed by the NanoSight software.

### Mass Spectrometry

Pulled-down immunocomplex from PMH protein samples, or EV lysate prepared as described above were run on a 4%-20% SDS-PAGE gel and fixed for 15 minutes with 50% methanol/10% acetic acid solution. The gel was subsequently washed for five minutes, stained with Coomassie blue (Bio-Rad) for one hour, washed for one hour, and then sectioned into six separate slices according to molecular weight. The gel pieces were destained in 50% acetonitrile/50 mM Tris pH 8.2 until clear, and the proteins reduced with 50 mM TCEP/50 mM Tris pH 8.2 at 60° C. for 30 minutes, followed with alkylation using 25 mM iodoacetamide/50 mM Tris pH 8.2 at room temperature for 30 minutes in the dark. Proteins were digested in-situ with 0.125 μg trypsin (Promega Corporation, Madison Wis.) in 25 mM Tris pH 8.2/0.0002% Zwittergent 3-16, at 37° C. overnight, followed by peptide extraction with 2% trifluoroacetic acid and acetonitrile. The pooled extracts were concentrated and the proteins were identified by nano-flow liquid chromatography electrospray tandem mass spectrometry (nanoLC-ESI-MS/MS) using a Thermo Scientific Q-Exactive Mass Spectrometer (Thermo Fisher Scientific, Bremen, Germany) coupled to a Thermo Ultimate 3000 RSLCnano HPLC system. The digest peptide mixture was loaded onto a Halo C18 2.7 μm EXP stem trap (Optimize Technologies, Oregon City, Oreg.) and chromatography was performed using 0.2% formic acid in both the A solvent (98% water/2% acetonitrile) and B solvent (80% acetonitrile/10% isopropanol/10% water), with a 5% B to 40% B gradient over 70 minutes at 400 nL/minute through a PicoFrit (New Objective, Woburn, Mass.) 100 μm×35 cm column handpacked with Agilent Poroshell 120 EC C18 packing. The Q-Exactive mass spectrometer experiment was a data dependent set up with a MS1 survey scan from 340-1500 m/z at resolution 70,000 (at 200 m/z), followed by HCD MS/MS scans on the top 15 ions having a charge state of +2, +3, or +4, at resolution 17,500. The ions selected for MS/MS were placed on an exclusion list for 30 seconds. The MS1 AGC target was set to 1e6 and the MS2 target was set to 1e5 with max ion inject times of 50 ms for both.

### Database Searching

Tandem mass spectra were extracted by msconvert version 3.0.9134. Charge state deconvolution and deisotoping was not performed. All MS/MS samples were analyzed using Mascot (Matrix Science, London, UK; version 2.4.0) and X! Tandem (The GPM, thegpm.org; version X! Tandem Sledgehammer (2013.09.01.1)). Mascot and X! Tandem were set up to search a current Swissprot mouse database with reverse decoy (33922 entries) assuming the digestion enzyme strict trypsin and with a fragment ion mass tolerance of 0.020 Da and a parent ion tolerance of 10.0 PPM. Glu->pyro-Glu of the n-terminus, ammonia-loss of the n-terminus, gln->pyro-Glu of the n-terminus, oxidation of methionine, phosphorylation of tyrosine were specified in X! Tandem as variable modifications and carbamidomethyl of cysteine was specified as a fixed modification. Oxidation of methionine and phosphorylation of tyrosine were specified in Mascot as variable modifications with carbamidomethyl of cysteine as fixed modifications.

### Criteria for Protein Identification

Scaffold (version Scaffold_4.8.3, Proteome Software Inc., Portland, Oreg.) was used to validate MS/MS based peptide and protein identifications. Peptide identifications were accepted if they could be established at greater than 95.0% probability by the Scaffold Local FDR algorithm. Protein identifications were accepted if they can be established at greater than 95.0% probability and contain at least two identified peptides. Protein probabilities were assigned by the Protein Prophet algorithm (Nesvizhskii et al. Anal. Chem., 75(17):4646-58 (2003)). The false discovery rate was less than 2%. Proteins that contained similar peptides and cannot be differentiated based on MS/MS analysis alone were grouped to satisfy the principles of parsimony. Proteins sharing significant peptide evidence were grouped into clusters. Protein comparisons were made with ratios of total spectral counts and considering total unique peptide counts.

### Immunoblot Analysis

Huh7 cells, AML12 cells, PMHs, and EVs were lysed using RIPA buffer (50 mM Tris-HCl, pH 7.4; 1% Nonidet P-40; 0.25% sodium deoxycholate; 150 mM NaCl; 1 mM EDTA with protease inhibitors) followed by centrifugation at 15,000 g for 15 minutes at 4° C. Protein concentrations of the lysates were measured by the Bradford assay method (Sigma-Aldrich). Equal amount of protein were loaded onto Sodium dodecyl sulfate (SDS)-Polyacrylamide gel electrophoresis (PAGE) gels, transferred to nitrocellulose membrane (Bio-Rad, Hercules, Calif.) and incubated overnight with the primary antibody of interest. All primary antibodies were used at a dilution of 1:1,000 unless otherwise recommended by the manufacturer. Horseradish peroxidase-conjugated secondary antibodies against rabbit (Alpha Diagnostic International, San Antonio, Tex.) or mouse (Southern Biotech, Birmingham, Ala.) were used at a dilution of 1:5,000 and incubated for 1 hour at room temperature. Proteins were detected using enhanced chemiluminescence reagents (GE Healthcare, Chicago, Ill.). β-actin and GAPDH protein levels, and EV markers CD81, CD63 and TSG101 protein levels were used as loading controls of whole cell lysates and EVs, respectively.

### Electron Microscopy and Immunogold Staining

The isolated EVs were fixed in 4% paraformaldehyde in 0.1 M phosphate buffer overnight at 4° C. The samples were then placed on a formvar-carbon-coated grid and air dried for 20 minutes. After being rinsed with PBS, grids were transferred to 1% glutaraldehyde for 5 minutes and washed with distilled water. The grids were first contrasted and embedded in a mixture of 4% uranyl acetate and 2% methylcellulose (1:9 ratio). For immunogold staining, grids were blocked with 10% FBS for 20 min followed by overnight incubation at 4° C. with a primary (anti-ITGβ₁ (9EG7) antibody diluted in 1:10 ratio in blocking solution). Next, grids were incubated with anti-rat IgG conjugated with 12 nm gold particles for 1 hour, rinsed with PBS, fixed in 1% glutaraldehyde for 5 minutes and washed with distilled water. The grids were then contrasted and embedded in a mixture of 4% uranyl acetate and 2% methylcellulose in 1:9 ratio. The grids were examined with a JEOL 1400 electron microscope (JEOL USA, Peabody, Mass.) at 80 kV.

### Immunoprecipitation and Pulldown Assays

Whole cell lysates (WCL) were obtained using immuno-precipitate (IP) buffer (50 mM Tris-HCl, pH 7.4; 1% Nonidet P-40; 150 mM NaCl) with protease inhibitor and phosphatase inhibitor followed by centrifugation at 15,000 g for 15 minutes at 4° C. Protein concentrations of the lysates were measured by the Bradford assay method (Sigma-Aldrich). In pull-down experiments, aliquots containing 1 mg of protein were incubated with either anti-ITGβ1 (MAB13), anti-ITGβ1 (9EG7) antibody or isotype IgG control antibody for 1 hour at 4° C., then incubated overnight with protein G sepharose beads (GE Healthcare) under rotary agitation. Pelleted proteins were washed with IP buffer for seven times, solubilized in SDS sample buffer, boiled for 10 minutes, and then subjected to SDS-PAGE and immunoblot analysis.

### Nanoscale Flow Cytometry

Cell culture medium from Huh7 or PMH, treated with vehicle or LPC 20 μM for 4 hours were collected and centrifuged at 2,000 g for 20 minutes to remove the cell debris. Supernatants were concentrated using ultrafiltration filter (Amicon Ultra-15, Millipore Sigma, USA) at 4,000 g for 30 minutes. Anti-ITGβ₁ (9EG7) was conjugated with Alexa Fluro-647 using conjugation kit (A20186, Thermo Fisher Scientific). To quantify ITGβ₁+ EVs in each condition, 50 μL of concentrated culture media were incubated with anti-ITGβ₁ (9EG7)-AF647 at 10 μg/mL for 30 minutes at room temperature in the dark. To control for non-specific binding and autofluorescence, samples were also incubated with rat IgG2a-APC isotype antibody. After incubation, the samples were then further diluted with PBS and analyzed using nanoscale flow cytometry. Active conformation sensitive ITGβ₁ antibody 9EG7-labeled EVs from Huh7 and PMH conditioned culture media were analyzed using the A50-Micro Plus nanoscale flow cytometry (Apogee FlowSystems Inc.), calibration was done using Silica nanoparticles of various sizes. Sample flow rate of 1.5 μL/minute was used for all measurements and the time of acquisition was held constant for all samples at 60 seconds to yield enough events. Data were analyzed using FlowJo v10.5 software. Detection settings were defined with isotype-matched controls to minimize unspecific signal and background noise. Antibody-labeled samples were run using same settings.

### Immunocytochemistry and Confocal Microscopy

Huh7 cells were seeded on fibronectin-coated coverslips and fixed with 4% paraformaldehyde following LPC treatment. After permeabilization using 0.2% TritonX-100, the cells were blocked using blocking buffer (5% bovine serum albumin, 0.1% glycine in PBS) for one hour at room temperature, and then incubated with the primary antibody of interest overnight at 4° C. Antibodies were diluted in PBS containing 5% bovine serum albumin at a dilution of 1:200. After washing, slides were incubated with corresponding secondary antibodies in the dark for one hour at room temperature. Slides were mounted using Prolong Gold Anti-fade reagent (Thermo Fisher Scientific), and then examined by fluorescent confocal microscopy equipped with an ultraviolet laser (LSM 780; Zeiss, Jena, Germany). ZEN 2.3 lite software (ZEISS) was used for acquiring images. ImageJ software (National Institute of Health, Bethesda, USA) was used to analyze images. Pearson's correlation coefficient of co-localization was employed to quantify the degree of colocalization between fluorophores. Ten to 20 random fields in each group were selected for quantification and statistical analysis.

### RNA Sequencing and Bioinformatics Analysis

RNA sequencing was performed on primary mouse monocytes stimulated with EVs derived from LPC or vehicle-treated PMH at the Mayo Clinic Genotyping Shared Resource facility. RNA libraries were prepared using 200 ng of total RNA according to manufacturer's instructions for the TruSeq RNA Sample Prep Kit v2 (Illumina, San Diego, Calif.). The concentration and size distribution of the completed libraries was determined using an Agilent Bioanalyzer DNA 1000 chip (Santa Clara, Calif.) and Qubit fluorometry (Invitrogen, Carlsbad, Calif.). Libraries were sequenced at 53 million to 90 million reads per sample following Illumina's standard protocol using the Illumina cBot and HiSeq 3000/4000 PE Cluster Kit. The flow cells were sequenced as 100×2 paired end reads on an Illumina HiSeq 4000 using HiSeq 3000/4000 sequencing kit and HCS v3.3.20 collection software. Base calling was performed using Illumina's RTA version 2.5.2. Ingenuity Pathway Analysis (IPA) software was used to analyze the data.

### Adhesion Assay

A flow-based shear stress adhesion assay was used to assess adhesion of monocytes to sinusoidal endothelial cells. Briefly, microfluidic chambers were sterilized, coated with collagen solution (0.2 mg/mL) for 4 hours at 37° C., washed with PBS, and then filled with culture media. For sinusoidal endothelial cells to form a monolayer prior to the adhesion assay, the cells were seeded in the chamber channel three days prior to the assay at a density of 10⁷ cell/mL, and cultured at 37° C. in a humidified atmosphere of 5% CO2. Primary mouse monocytes or THP-1 cells at a density of 1×10⁶/mL were incubated overnight with EVs derived from LPC or vehicle-treated hepatocytes, and then incubated with or without ITGβ1 neutralizing antibody (Hmb1-1, eBioscience, San Diego, Calif.) (30 μg/mL) or ITGα₉ neutralizing antibody (Y9A2, Millipore Sigma) (10 μg/ml) for 30 minutes, re-suspended in serum-free RPMI-1640. To block the ITG β₁ ligand on the endothelial cells, primary human LSEC were incubated with VCAM-1 neutralizing antibody (R&D) (50 μg/mL) for 60 minutes. After infusing endothelial basal medium in the microfluidic chamber to remove any debris, 400 μL of the monocytes suspension were infused through the chamber at a constant shear stress of 0.01 Pa for 5 minutes. Then a video of 20 seconds was taken using a Lionheart FX automated live cell microscope (Bio Tek, Winooski, Vt.). To remove non-adherent monocytes, the chamber was infused with endothelial basal medium for 5 minutes. Images of 4-6 random fields were taken with 4×/10× objective magnification. Adherent monocytes were quantified using ImageJ software.

### Animals

Study protocols were conducted as approved by the Institutional Animal Care and Use Committee (IACUC) of Mayo Clinic. The methods employed in the current study were carried out in accordance with IACUC guidelines for the use of anesthetics in experimental mice. C57BL/6J male mice were purchased from Jackson Laboratory (Bar Harbor, Me.). Mice were housed and bred in a temperature-controlled 12:12-hour light-dark cycle facility with free access to diet. The mice were fed either a chow diet (5053 PicoLab Rodent Diet 20, LabDiet, St. Louis, Mo.) or a diet rich in fat, fructose, and cholesterol (FFC) at 8-weeks old for 24 weeks. FFC diet consists of 40% energy as fat (12% saturated fatty acid, 0.2% cholesterol) (AIN-76A Western Diet, TestDiet, St Louis, Mo.), with fructose (23.1 g/L) and glucose (18.9 g/L) in the drinking water. The FFC diet induces steatohepatitis with pronounced hepatocellular ballooning, lipoapoptosis, and progressive fibrosis with a high fidelity to the human NASH histology and metabolic profile (see, e.g., Charlton et al., Am J Physiol Gastrointest Liver Physiol, 301(5):G825-34 (2011); Ibrahim et al., Dig Dis Sci, 61(5):1325-36 (2016); Krishnan et al., Am J Physiol Gastrointest Liver Physiol, 312(6):G666-G680 (2017); and Idrissova et al., Journal of Hepatology, 62(5):1156-1163 (2015)). At 20 weeks on the diet, the mice were randomized to receive either anti-ITGβ₁ neutralization antibody (Hmb1-1) (16-0291-85, eBioscience) or IgG isotype antibody (14-4888-85, eBioscience). Mice were injected with 1 μg/g body weight of either of the antibodies intraperitoneally, twice per week for 4 weeks. At third week of antibody treatment, metabolic parameters, including food intake, oxygen consumption, carbon dioxide production, and locomotor activity, were measured using a Comprehensive Lab Animal Monitoring System (CLAMS, Columbus Instruments, OH). Blood glucose levels and plasma insulin levels were measured using Assure 4 (Arkray, Edina, Minn.) and Ultra-Sensitive Mouse Insulin enzyme-linked immunosorbent assay (ELISA) kit (Crystal Chem Inc., Downers Grove, Ill.), respectively. Homeostasis model assessment of insulin resistance (HOMA-IR) was calculated by using the following formula: HOMA-IR=26× fasting insulin level (ng/mL)×fasting glucose level (mg/dL)/405. Mice were sacrificed under general anesthesia induced by a ketamine/xylazine cocktail (83 mg/kg ketamine, 16 mg/kg xylazine, intraperitoneal). All interventions, including sacrifice, were made during the light cycle. Blood, and liver samples were collected for further study.

### Mouse Model to Track EVs of Hepatocyte Origin

Gt(ROSA)26Sor^{tm4(ACTB-tdTomato,-EGFP)Luo}/j mice (Jackson Lab, cat #007676) have a cell membrane targeted two-color fluorescent Cre-reporter allele that highlights the cytoplasmic membrane and other cellular membranous structures, allowing the fluorescent labelling of both exosomes and microvesicles. Cre recombinase expressing cells (and future cell lineages derived from these cells) have cell membrane-localized EGFP (mG) fluorescence expression replacing the red fluorescence. These mice are referred to as red-green mice (FIG. 4). Adult male mice were fed either chow or FFC diet for 16 weeks. At week 9 on the diet, these mice were injected through tail vein with 1.45×10¹¹ GC of the Adeno-Associated Viral Vector (AAV) 8 TBG.PI.Cre.rBG (Penn vector core)/mouse to generate GFP+ hepatocytes. The AAV8 serotype was chosen for its hepatotropism and the thyroxine binding globulin (TBG) promoter to assure efficient and sustained cre expression in hepatocytes. AAV8.TBG.PI.Null.bgh was employed as control. To prepare platelet-free plasma (PFP), the red-green mouse blood was collected using heparin blood vacutainers (BD Biosciences Inc.) and centrifuged at 2,500 g for 20 minutes (at room temperature, with no brake), twice. Quantification of GFP+ EVs in the plasma was then performed by nanoscale flow cytometry as described above.

### Liver Triglyceride and Alanine Aminotransferase Measurement

Liver triglyceride levels were measured in mouse liver homogenates. Fifty milligrams of liver tissue was homogenized in a 5% NP-40 solution. EnzyChrom Triglyceride Kit (BioAssay System, CA) was used for the assay according to the manufacturer's instruction. Photometric absorbance was read at 570 nm using a Synergy H1 microplate reader (BioTek). Serum alanine aminotransferase (ALT) levels were measured by VetScan2 (Abaxis Veterinary Diagnostics, Union City, Calif.).

### Histology, Immunohistochemistry, and Digital Image Analysis

Liver histology was performed using tissue fixed in 10% formalin, dehydrated, and embedded in paraffin. Hematoxylin and eosin (H&E) staining and Sirius red staining were performed. Histology was assessed using nonalcoholic fatty liver disease (NAFLD) activity score (NAS), a semi-quantitative score that accounts for steatosis, ballooned hepatocytes, and lobular inflammation. Terminal deoxynucleotidyl transferase deoxyuridine triphosphate nick-end labeling (TUNEL) assay was performed using the ApopTag Peroxidase In Situ Apoptosis Detection Kit (Millipore Sigma, St. Louis, Mo.); diaminobenzidine (DAB) was used as a peroxidase substrate (Vector Laboratories, Burlingame, Calif.); 0.5% methyl green was used for the counterstain. In the immunohistochemistry studies, formalin-fixed paraffin-embedded liver tissue sections were deparaffinized, hydrated, and stained with antibody against ITCβ1 (1:1000), Mac-2 (1:250), or alpha smooth muscle actin (α-SMA) (1:1000). Bound antibodies were detected using a Vectastain ABC kit (Vector Laboratories) and DAB substrate (Vector Laboratories) according to the manufacturer's instructions; the tissue sections were counterstained with hematoxylin. Sirius red-positive, ITCβ1, Mac-2, or α-SMA-positive areas were quantified by digital image analysis of 10 random fields per slide per animal using the ImageJ software. TUNEL-positive cells were quantified by counting positive nuclei in 10 random fields per slide per animal.

### Quantitative Real-Time PCR

Total RNA was isolated with the RNeasy Mini Kit (Qiagen, Valencia, Calif.) and was reverse transcribed with moloney murine leukemia virus reverse transcriptase and oligo-dT random primers (both from Invitrogen, CA, USA). Quantification of gene expression was performed by real-time PCR using SYBR green fluorescence on a LightCycler 480 instrument (Roche Applied, IN, USA) (Primers are listed in Table 4). Target gene expression was calculated using the ΔΔCt method and was normalized to 18s rRNA expression levels, which were stable across experimental groups.

TABLE 4
Primers used for qRT-PCR

| | Forward | SEQ | Reverse | SEQ |
|---|---|---|---|---|
| | Primer | ID | Primer | ID |
| Gene | (5′-3′) | NO: | (5′-3′) | NO: |
| Cd68 | TGTCTGATCT | 2 | GAGAGTAACGG | 3 |
| | TGCTAGGACG | | CCTTTTTGTGA | |
| Ccr2 | ATCCACGGCA | 4 | CAAGGCTCACC | 5 |
| | TACTATCAAC | | ATCATCGTAG | |
| | ATC | | | |
| Tnf-α | CCCTCACACT | 6 | GCTACGACGTG | 7 |
| | CAGATCATCT | | GGCTACAG | |
| | TCT | | | |
| Collagen1α1 | GCTCCTCTTA | 8 | CCACGTCTCAC | 9 |
| | GGGGCCACT | | CATTGGGG | |
| Osteopontin | CTCCATCGTC | 10 | TGCACCCAGAT | 11 |
| | ATCATCATCG | | CCTATAGCC | |
| 18s | CGCTTCCTTA | 12 | GAGCGACCAAA | 13 |
| | CCTGGTTGAT | | GGAACCATA | |

### Flow Cytometry

One gram of liver tissue was dissociated using mouse liver dissociation kit (Miltenyi Biotec, Bergisch Gladbach, Germany) according to the manufacturer's instruction. Intrahepatic leukocytes were purified by Percoll (Sigma-Aldrich) gradient centrifugation. Cells were stained with the following antibodies for 20 minutes at room temperature in the dark: anti-CD45-Vio-green (130-110-665, Miltenyi Biotec), anti-CD11b-PerCp-vio700 (130-109-289, Miltenyi Biotec), anti-F4/80-APC-Cy7 (123117, Biolegend, San Diego, Calif.), anti-CCR2-PE (130-108-724, Miltenyi Biotec), anti-CLEC4F (370901, R&D) conjugated with Alexa Fluro-647 using conjugation kit (A20186, Thermo Fisher Scientific). Stained cells were analyzed on MACSQuant Analyzer 16 Flow Cytometer (Miltenyi Biotec). Data were analyzed using FlowJo v10.5 software.

### Mass Cytometry by Time-of-Flight (CyTOF) Analysis

Intrahepatic mouse leukocytes were isolated using liver dissociation kit and Percoll gradient centrifugation as described above. Cells were suspended in Maxpar Cell Staining Buffer (CSB, Fluidigm, San Francisco, Calif.) and labelled with 0.5 μM cisplatin (Fluidigm) solution. After centrifugation, cells were resuspended in CSB prior to addition of the antibody cocktail (composition shown in Table 5) in an equal volume of CSB. Cells were incubated with gentle agitation at room temperature for 45 minutes. Following wash, cells were fixed overnight with 2% paraformaldehyde with gentle agitation at 4° C. DNA intercalation was performed by adding 1:10000 diluted 125 μM of Cell-ID™ Intercalator-Ir (Fluidigm) with gentle agitation in 4° C. for 30 minutes. Cells were resuspended in 1:10 dilution of EQ beads (EQ Four Element Calibration Beads, Fluidigm), and then loaded onto Helios sample loader for data acquisition. Mass cytometry was performed in the Immune Monitoring Core at Mayo Clinic, and employed antibodies conjugated to stable heavy-metal isotopes to detect cellular antigens by mass cytometry time-of-flight (CyTOF) and enables comprehensive profiling of the phenotype and function of the intrahepatic leukocytes. After data acquisition, fcs files were normalized using CyTOF Software (version 6.7.1014). Cleanup of cell debris, removal of doublets and dead cells was performed using FlowJo software version 10.5.3 (Ashland, Oreg.). Cleaned fcs files were analyzed by the R-based tool Cytofkit version 3.8. Clustering and dimensionality reduction to 20,000 events per file was performed using the Rphenograph algorhithm that included all 24 markers in the panel (Table 5). Visualization of clusters was mapped onto a tSNE map. Relative marker intensities and cluster abundances per sample were visualized by heatmap.

TABLE 5
CyTOF Panel.

| Species | | | |
|---|---|---|---|
| Specificity | Label | Target | Clone |
| Mouse | 150Nd | I-A/I-E | M5/114.15.2 |
| Mouse | 143Nd | TCRb | H57-597 |
| Mouse | 144Nd | MHC Class I | 28-14-8 |
| Mouse | 152Sm | CD3e | 145-2C11 |
| Mouse | 161Dy | Ly6G | 1A8 |
| Mouse | 164Dy | CX3CR1 | SA011F11 |
| Mouse | 168Er | CD8a | 53-6.7 |
| Mouse | 172Yb | CD11b (Mac-1) | M1/70 |
| Mouse | 175Lu | Ly6C | HK1.4 |
| Mouse | 089Y | CD45 | 30-F11 |
| Mouse | 166Er | CD19 | 6D5 |
| Mouse | 142Nd | CD11c | N418 |
| Mouse | 154Sm | TER-119 | TER-119 |
| Mouse | 156Gd | CCR2 | 475301 |
| Mouse | 159Tb | F4/80 | BM8 |
| Mouse | 170Er | CD161 (NK1.1) | PK136 |
| Mouse | 174Yb | CD115/CSF1R | AFS98 |
| Mouse | 176Yb | CD45R (B220) | RA3-6B2 |
| Mouse | 141Pr | Lgals3 | 202213 |
| Mouse | 151Eu | CD206 (MMR) | C068C2 |
| Mouse | 155Gd | MERTK | 108928 |
| Mouse | 160Gd | CD64 | 290322 |
| Mouse | 165Ho | CD14 | Sa14-2 |
| Mouse | 149Sm | Tim4 | 370901 |

Immunostaining of EVs from Patient Serum

De-identified archived serum samples from 8 patients with simple steatosis and 17 patients with stage I-II NASH were acquired through a human study that was approved by the Institutional Review Board of the Mayo Clinic and previously published. All subjects provided written informed consent. The diagnosis of NASH was based on histologic categorization according to established NASH criteria as assessed by experienced pathologist. Subjects with secondary causes of steatohepatitis (drugs, and prior gastric surgery for obesity) and other chronic liver diseases (cholestatic liver disease, hemochromatosis, excessive alcohol consumption, viral hepatitis (B, C), Wilson disease, drug-induced liver disease, and alpha-1-antitrypsin deficiency) were excluded. Sera were pre-cleared by centrifuging at 2,500 g for 15 minutes, twice, prior to nanoscale flow cytometry staining; 10 μL serum was co-stained with anti-CD29-APC and ASGR1-PE or isotype antibodies at 10 μg/mL for 30 minutes at room temperature in the dark. After incubation, the samples were then further diluted with PBS and analyzed using nanoscale flow cytometry as described above.

### Statistical Analysis

Data are expressed as the means±SEM. Differences between multiple groups were compared using one-way analysis of variance followed by Bonferroni's multiple comparisons test or Student t test when comparing two groups. *, **, ***, **** indicate statistical significance with p<0.05, p<0.01, p<0.001 and p<0.0001 respectively. Statistically non-significant results were labelled as ns where appropriate. All analyses were performed using GraphPad Prism 8 software (CA, USA).

### Results

Lipotoxic Hepatocyte-Derived EVs are Enriched with Integrins

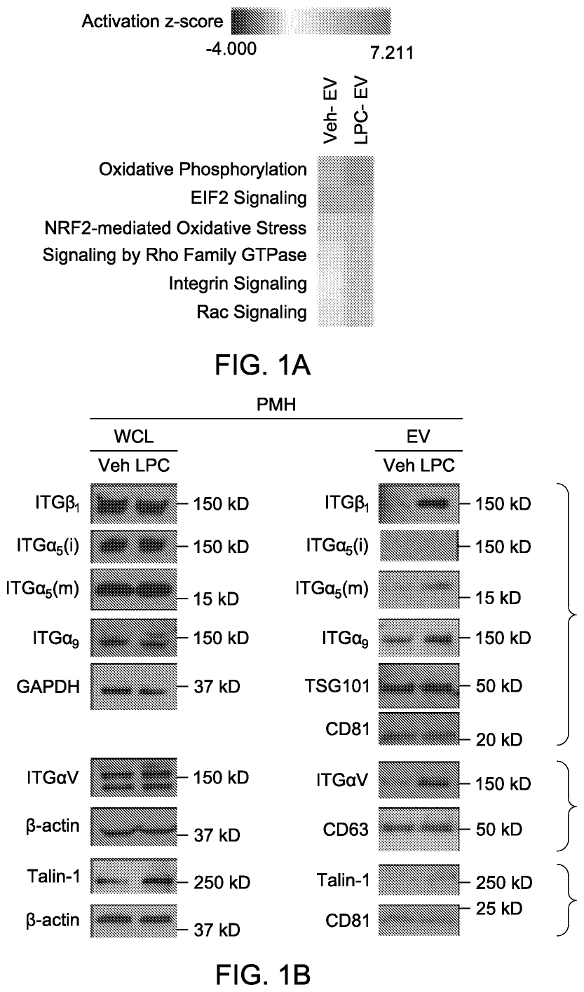

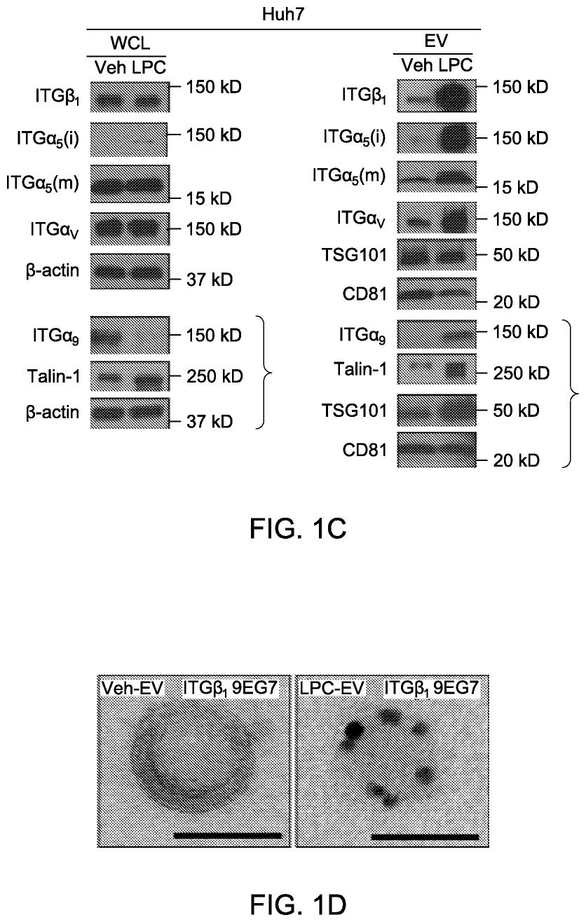

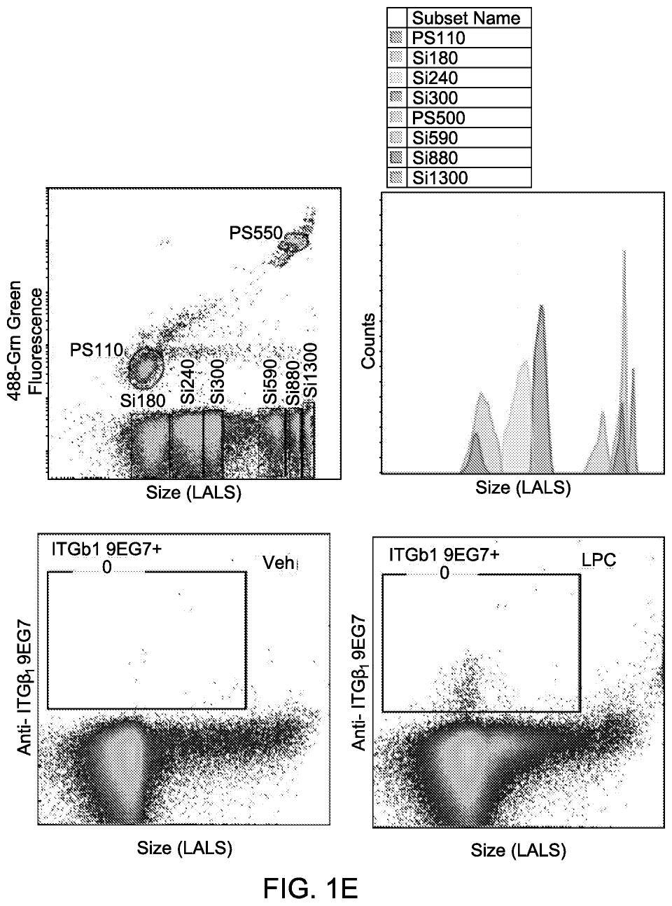

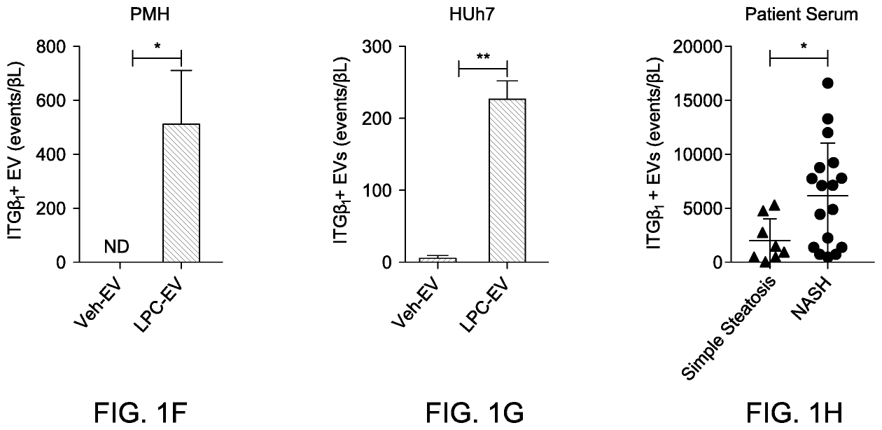

A non-biased approach was adopted to identify and characterize the key proteins on lipotoxic hepatocyte-derived extracellular vesicles (EVs). To this end, proteomics analysis was performed by mass spectrometry (MS) on the EVs derived from primary mouse hepatocytes (PMH) treated with vehicle (Veh) and the toxic lipid mediator lysophosphatidylcholine (LPC). LPC was employed since the toxicity of the saturated free fatty acid palmitate is dependent upon its metabolism to LPC. Unbiased Ingenuity pathway analysis (IPA) of the proteomics data identified ITG signaling among the top represented canonical pathways, particularly in EVs from LPC-treated hepatocytes when compared to EVs from vehicle-treated hepatocytes (FIG. 1A). Next, immunoblot analysis was performed for different ITG in hepatocytes treated with vehicle and LPC, and their derived EVs. Western blot identified selective enrichment of ITGβ₁, ITGα₅, ITGα₉, and ITGαᵥ in EVs released from lipotoxic PMH, without changes at the cellular levels (FIG. 1B). Similar results were obtained with the human hepatoma cell line Huh7 (FIG. 1C). Since ITGβ₁ is the most abundant integrin on hepatocytes and the only integrin β expressed on EVs based on the mass spectrometry data, ITGβ₁ was focused on as the key functional integrin family member on lipotoxic EVs. The protein level of Talin-1 (a versatile ITGβ₁ affinity regulator implicated in adhesion), was also increased in lipotoxic EVs, suggesting that the ITGβ₁ on lipotoxic EVs is in active conformation status. Immunogold electron microscopy was used to demonstrate the active conformation sensitive ITGβ1 antibody (9EG7) enrichment of ITGβ₁ in EVs released from lipotoxic PMH (FIG. 1D). This observation was further confirmed by nanoscale flow cytometry, which allows the quantification of active ITGβ₁-bearing EVs. LPC-treated PMH released more abundant active ITGβ₁-positive EVs as compared to Veh-treated PMH (FIG. 1F). These findings were also confirmed using Huh7 cells (FIG. 1G). ITGβ1 expression was increased in the serum EVs of patients with NASH (FIG. 1H). Collectively, these data indicate that ITGβ₁ in an active conformation is selectively sorted into EVs released from lipotoxic hepatocytes.

### Hepatocyte Lipotoxic Treatment Induces ITGβ₁ Activation and Endocytic Trafficking

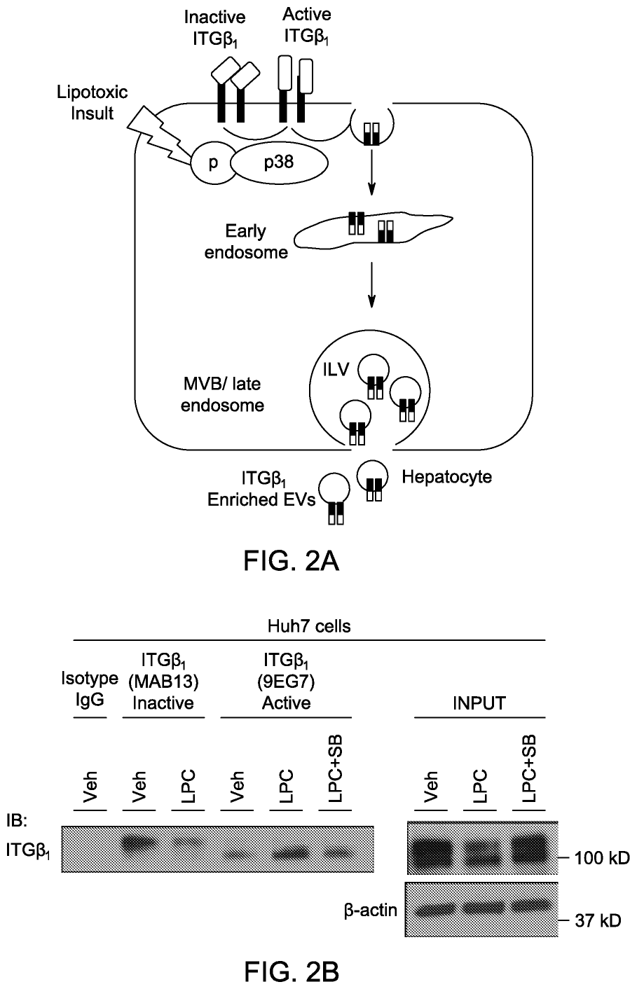

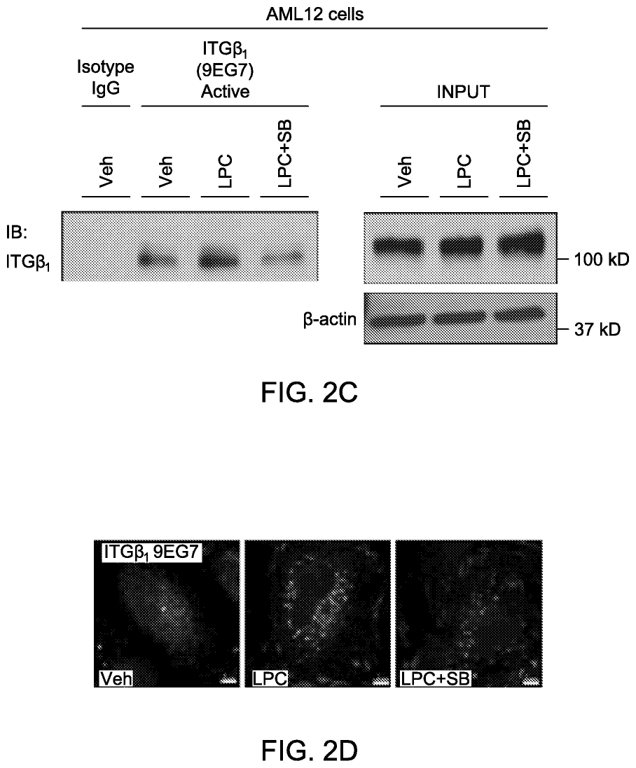

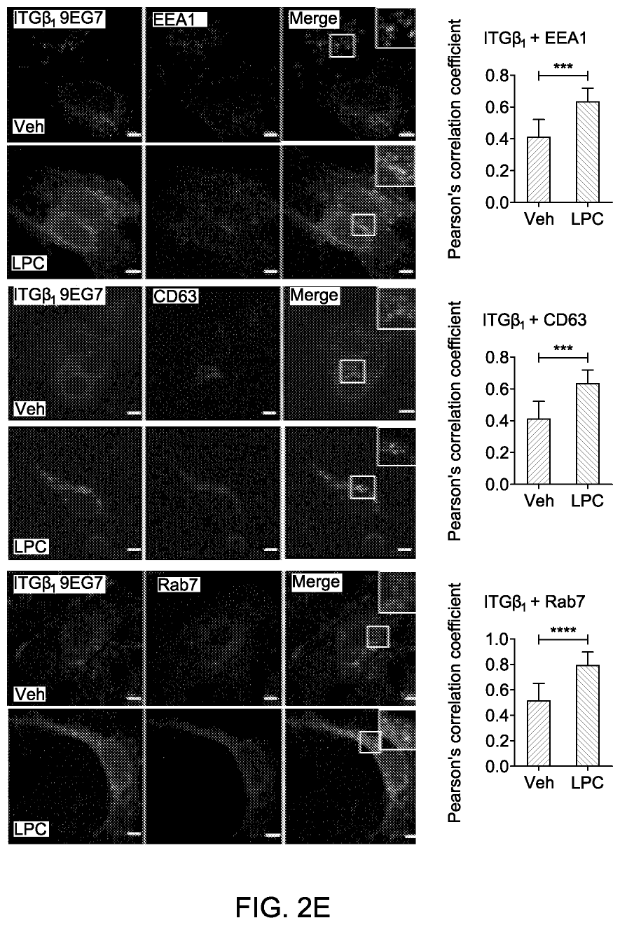

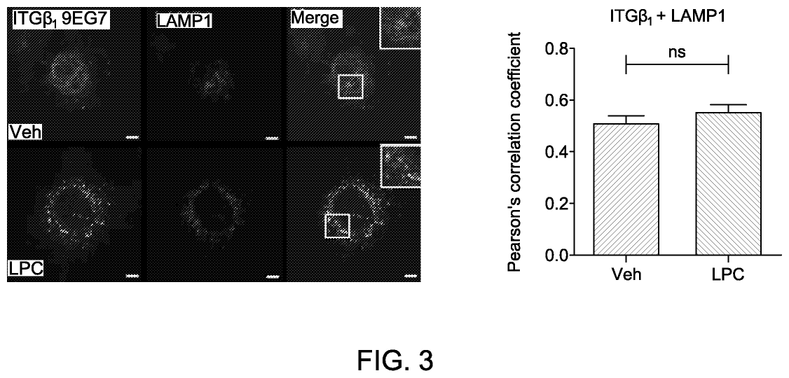

The activation and endocytic trafficking of ITGβ₁ in hepatocytes under lipotoxic stress was examined (FIG. 2A). Conformation-specific antibodies against ITGβ₁ cannot detect the SDS-denatured target protein, and thus are not suitable for immunoblot assay. Therefore, to determine if hepatocyte ITGβ₁ is activated by lipotoxic treatment, lysates from Veh or LPC-treated Huh7 cells were immunoprecipitated with the inactive (MAB13) or the active (9EG7) conformation-specific ITGβ₁ antibodies, and immunoblotted with an antibody for total ITGβ₁ (FIG. 2B). Whether p38 mediates LPC-induced ITGβ₁ activation in hepatocyte was investigated. LPC treatment causes a significant decrease in inactive (tyrosine phosphorylated ITGβ₁ tail, higher molecular weight), and increase in active (tyrosine dephosphorylated tail, lower molecular weight) ITGβ₁, which is reduced in the presence of the p38 inhibitor SB203580, indicating that lipotoxic stress-induced hepatocyte ITGβ₁ activation occurs via a p38-mediated pathway. Similar results were obtained with the mouse hepatocyte cell line AML12 (FIG. 2C), (the inactive conformation sensitive antibody MAB13 reacts only with human species hence it was not used with the mouse AML12 cells). To confirm that the ITGβ₁ 9EG7 antibody immunoprecipitates active ITGβ₁, the ITGβ₁ immunoprecipitates from vehicle- and LPC-treated PMH were analyzed by MS. The MS data was searched allowing for phosphostyrosine as a variable modification and showed absence of phosphorylation on the first NPxY motif on the ITGβ₁ C terminal, confirming that the pulled ITGβ₁ is in an active conformation. Immunofluorescence (IF) microscopy was employed and it was confirmed that LPC treatment induced activation of ITGβ₁, which was diminished with SB203580 (FIG. 2D). Moreover, it was noted that in lipotoxic hepatocytes, the active ITGβ₁ accumulated in cytoplasmic structures consistent with intracellular vesicles (FIG. 2D, second panel). To further explore the intracellular trafficking of active ITGβ₁, the co-localization of active ITGβ₁ with the early endosome marker early endosome antigen (EEA) 1, the MVB marker CD63, and the late endosome marker Rab7 were examined. ITGβ₁ co-localization with EEA1, CD63 or Rab7 (FIG. 2E) was increased with LPC treatment when quantified using the Pearson's correlation coefficient. To examine if lipotoxicity regulates active ITGβ₁ lysosomal degradation, the co-localization of active ITGβ₁ with the lysosome marker LAMP1 was assessed; however, there was no obvious co-localization of ITGβ₁ with LAMP1 (FIG. 3). Collectively, these results suggest that hepatocyte lipotoxic treatment induces ITGβ₁ activation and endocytic trafficking, resulting in active ITGβ1 release in EVs.

### Lipotoxic Hepatocytes Release EVs in the Circulation

A mouse model to track circulating EVs of hepatocyte origin was developed (FIG. 4A). The hepatocyte-derived EVs in the plasma were quantified by nanoscale flowcyometry, and identified a 10-fold increase with the NASH-inducing diet (FIG. 4B). These data conclusively demonstrate for the first time that lipotoxic hepatocytes release large number of EVs in the circulation in vivo.

### Lipotoxic Hepatocyte-Derived EVs Promote Monocytes Adhesion to LSECs Via an ITGβ₁-Dependent Mechanism

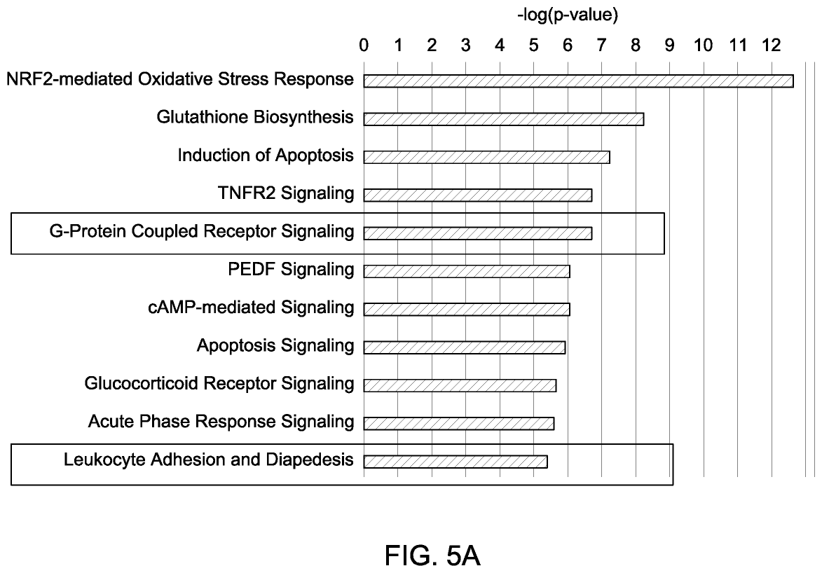

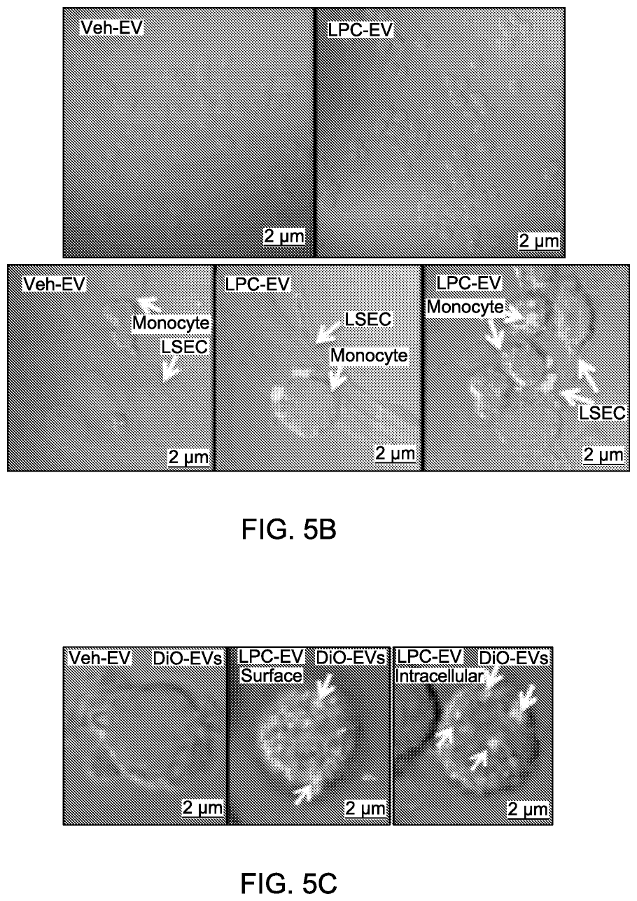

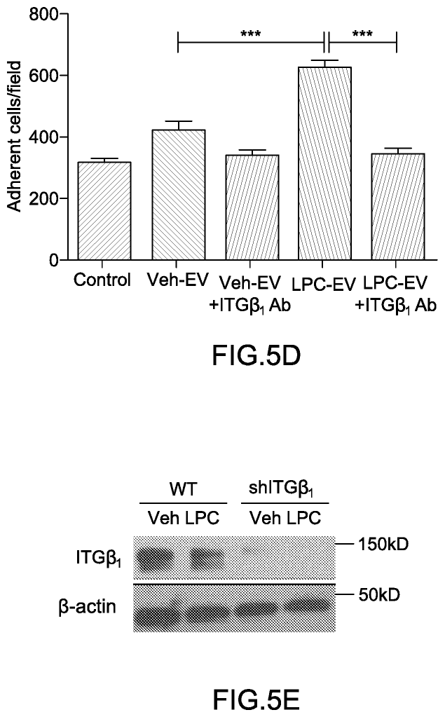

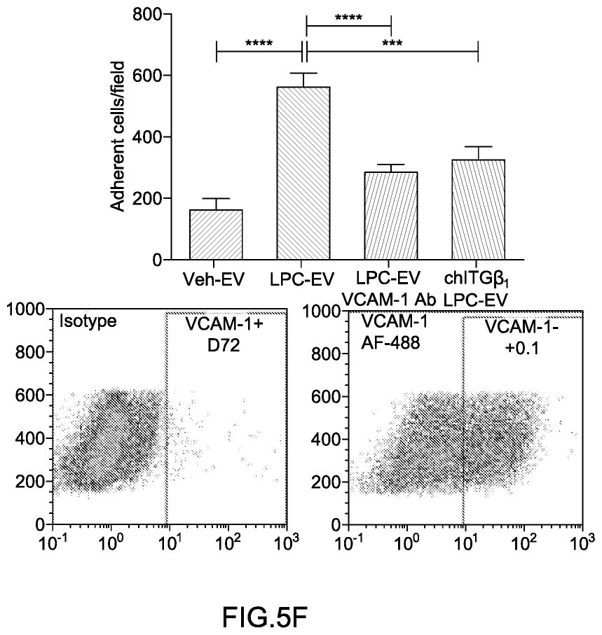

To understand the biological functions exerted by lipotoxic hepatocyte-derived EVs on monocytes, RNA sequencing (RNAseq) was performed on primary mouse monocytes incubated with EVs from LPC (LPC-EVs)- or vehicle (Veh-EVs)-treated PMH. Ingenuity pathway analysis of RNAseq data showed that leukocyte adhesion and diapedesis-related signaling among the top overrepresented canonical pathways in monocytes stimulated with LPC-EVs, suggesting the involvement of LPC-EVs in monocyte adhesion to LSECs (FIG. 5A).

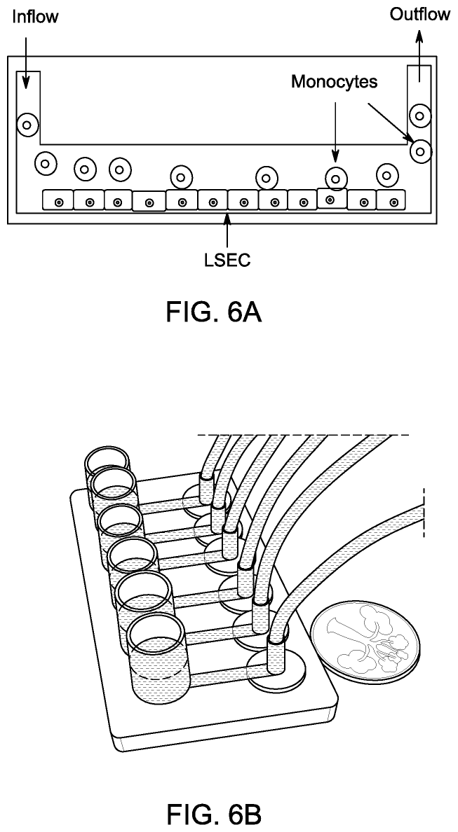

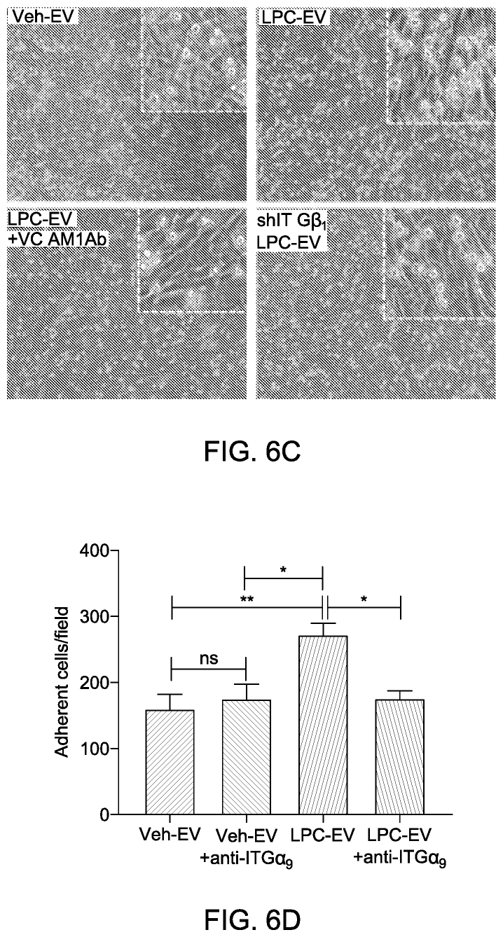

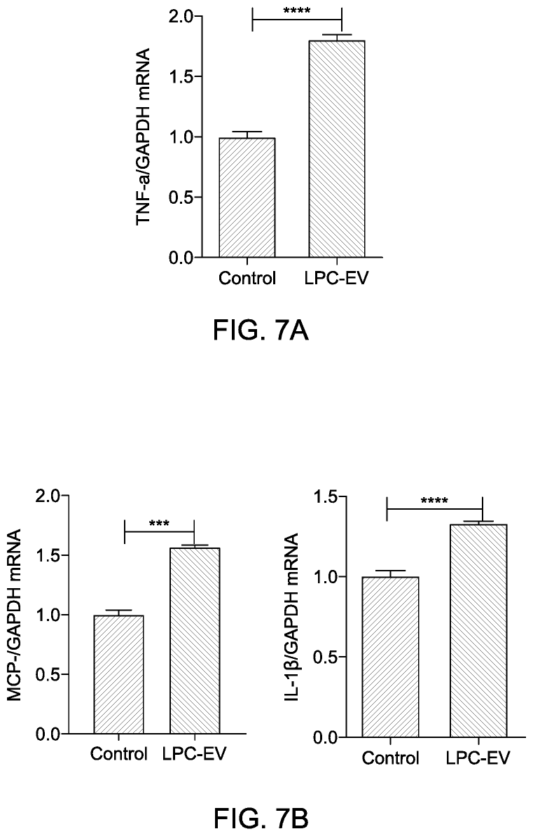

To examine the interaction between lipotoxic EVs-stimulated monocytes and LSECs, the human monocyte cell line, THP1, was co-cultured with EVs derived from equal number of hepatocytes treated with either vehicle or LPC. EVs were labelled with a fluorescent lipophilic dye DiO. Confocal microscopy revealed that monocytes incubated with LPC-EVs were more likely to adhere to LSECs (FIG. 5B). Monocytes were then subjected to live cell imaging with Z stack microscopy following incubation with EVs. EVs were observed both on the surface and on deeper focal plane (intracellular) of the THP1 cells (FIG. 5C), suggesting that ITGβ₁-enriched EVs interact with monocytes in a topography that allows them to potentially tether monocytes to LSECs. Interestingly, many of these EVs are also internalized by monocytes (FIG. 5C), which has implications for ITGβ₁ recycling to the cell surface. To further explore if LPC-EVs enriched with ITGβ₁ mediates monocyte adhesion to LSECs, a key stage in liver inflammation, a flow-based adhesion assay was employed using microfluidic chambers (FIG. 6A-B) coated with a monolayer of mouse primary LSECs. Monocytes stimulated with LPC-EVs have enhanced adhesion to LSECs (FIG. 5D). Interestingly this enhanced adhesion was diminished when monocytes were incubated with anti-ITGβ₁ neutralizing antibody (ITGβ₁Ab), suggesting that ITGβ₁ on lipotoxic EVs may be responsible for EV-induced monocyte adhesion to LSEC. This finding was confirmed using ITGβ₁-knockdown Huh7 by shRNA technology (shIβ₁) (FIG. 5E-F, FIG. 6C). Likewise, the adhesion of lipotoxic EVs-stimulated monocytes to endothelial cells was reduced in the presence of ITGα9 neutralizing antibody (FIG. 6D). Moreover, pretreatment of LSECs with a neutralizing antibody against VCAM-1 (an ITGα₉β₁ ligand expressed on LSECs under basal conditions as examined by flowcytometry), significantly diminished the adhesion of LPC-EVs-stimulated monocytes to LSECs (FIG. 5F). Taken together, these results support a role for LPC-EV ITGα₉β₁ in monocyte adhesion to LSEC via an ITGα₉β₁-VCAM-1 binding interactions. Monocyte inflammatory activation markers expression also was enhanced with lipotoxic EVs stimulation (FIG. 7), supporting the proinflammatory role of lipotoxic EVs.

Anti-ITGβ₁ Antibody Treatment does not Alter the Metabolic Phenotype or the Steatosis in FFC Diet-Fed Mice

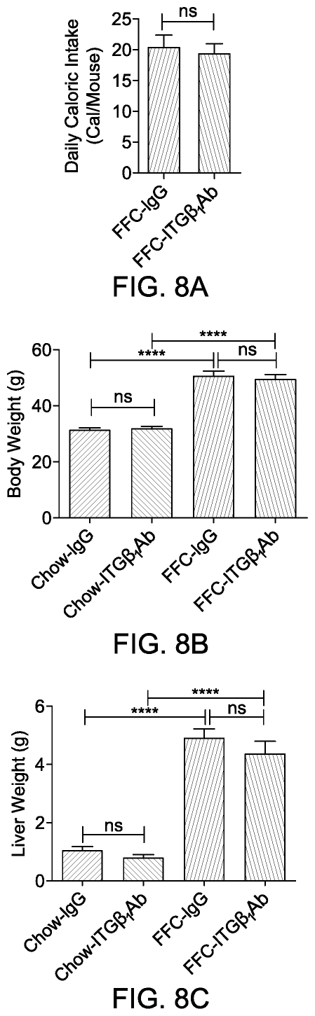

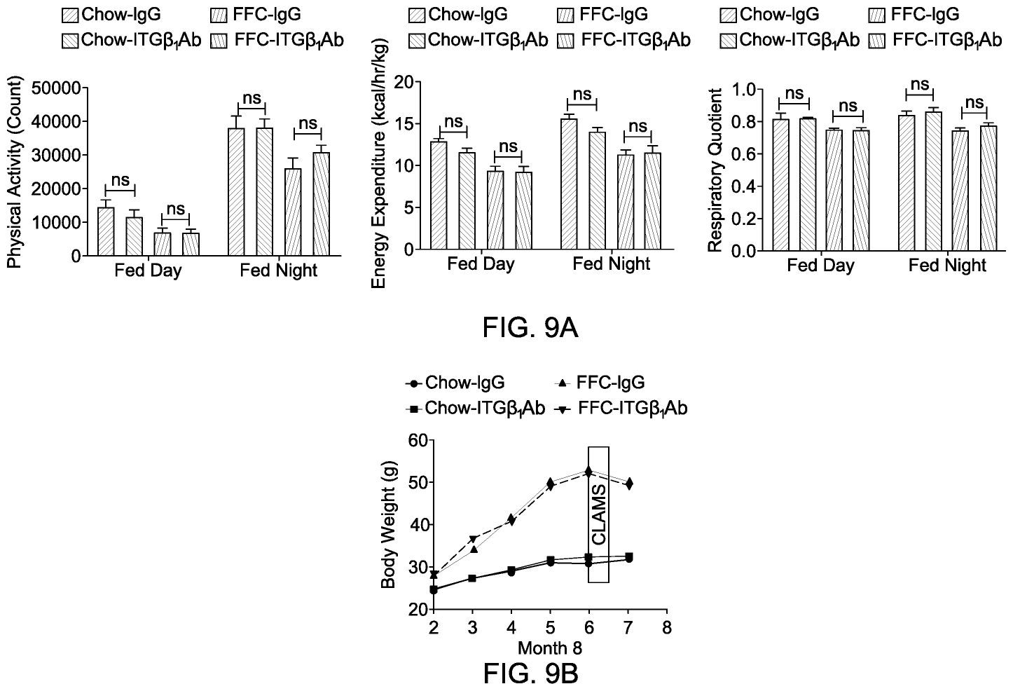

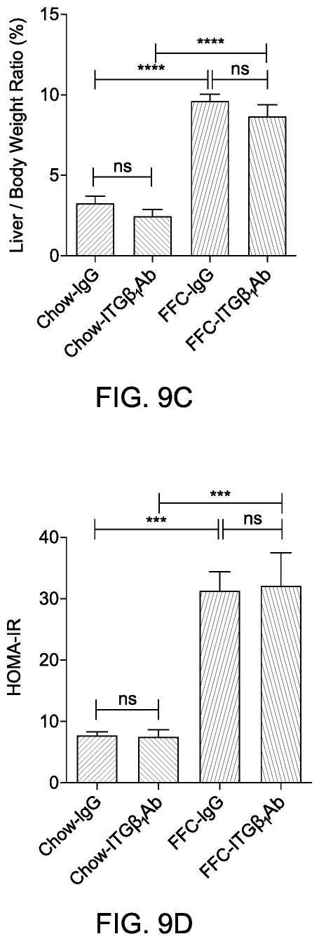

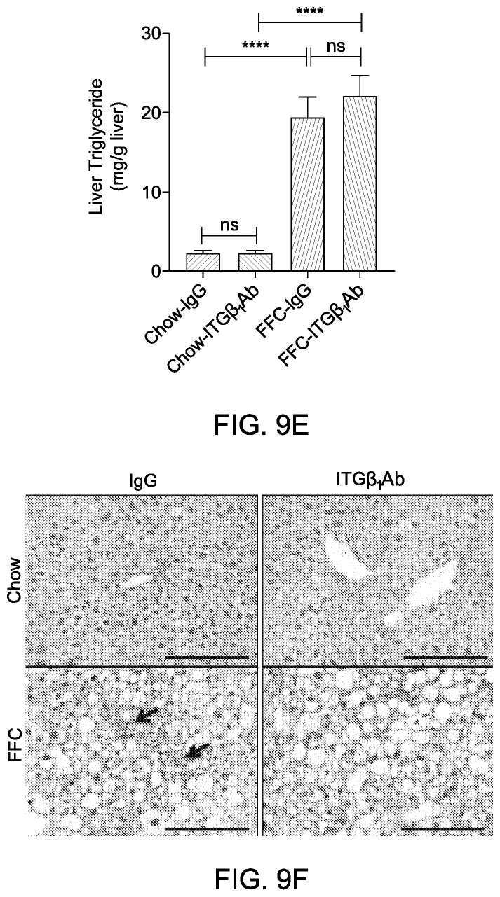

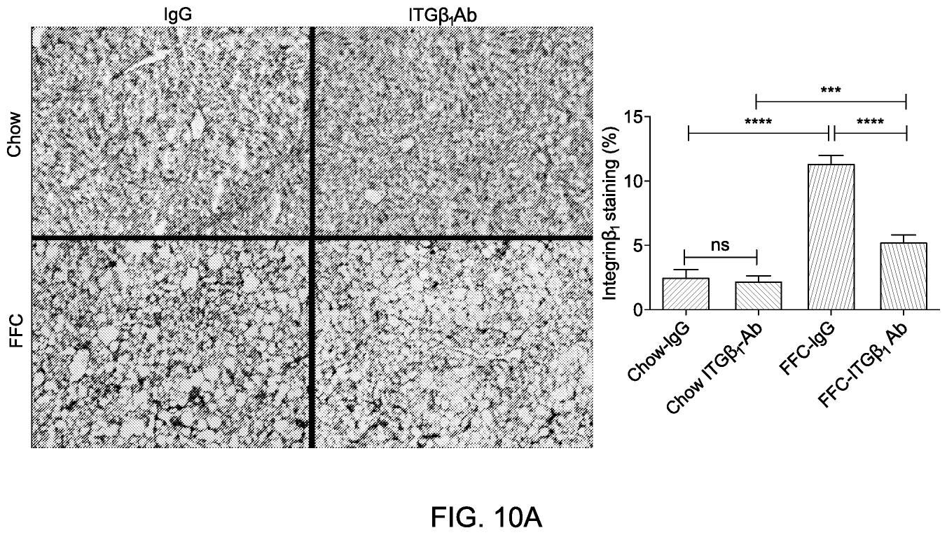

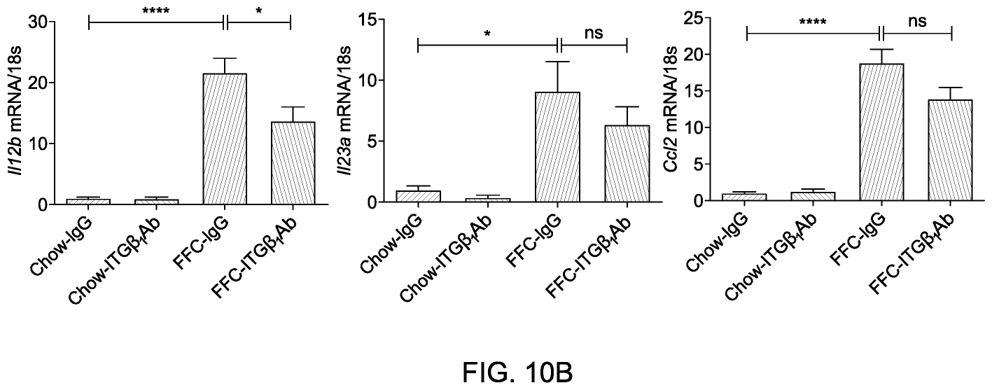

Based on the in vitro findings supporting a key role of lipotoxic EV ITGβ₁ in monocyte adhesion to LSECs and potentially in liver inflammation; the potential beneficial effect of ITGβ₁ neutralizing antibody in a mouse model of diet-induced NASH was examined. Eight-week-old C57BL/6J wild-type mice were fed either chow or a diet high in saturated fat, fructose, and cholesterol (FFC) for 24 weeks. At 20 weeks of the diet mice were treated with either anti-ITGβ₁ neutralizing antibody (ITGβ₁Ab) or control IgG isotype antibody (IgG) twice per week for 4 weeks. The metabolic status of each group of mice was assessed. Comprehensive Laboratory Animal Monitoring System (CLAMS) study showed that total daily caloric intake (FIG. 8A), physical activity, energy expenditure, and respiratory quotient (FIG. 9A) were similar between FFC-fed ITGβ₁Ab-treated versus control IgG-treated mice. Body weight during the whole study period (FIG. 9B, FIG. 8B), liver weight (FIG. 8C), and liver to body weight ratio (FIG. 9C) at the time of sacrifice were significantly increased with the FFC diet, but similar between ITGβ₁Ab-treated and control IgG-treated groups. Likewise, homeostasis model assessment of insulin resistance (HOMA-IR) (FIG. 9D), and triglyceride content in liver tissue (FIG. 9E) were increased with the FFC diet, but were not different between the 2 treatment groups on the FFC diet although HOMA-IR has some limitations in assessing insulin sensitivity in vivo. Moreover, histological examination of the liver by hematoxylin and eosin (H&E) stain displayed similar extent of steatosis in the FFC-fed mice from the different treatment groups (FIG. 9F). Interestingly, ITGβ₁Ab-treated mice had less inflammatory infiltrates compared to IgG-treated, FFC-fed mice (FIG. 9F). Consistent with the in vitro data, active ITGβ₁ expression was increased with FFC diet when assessed by immunohistochemistry and reduced with the ITGβ₁ neutralizing Ab (FIG. 10A). Collectively, ITGβ₁Ab treatment in FFC-fed mice was well tolerated, and did not affect the metabolic phenotype or the hepatic steatosis.

### Anti-ITGβ₁ Antibody Treatment in FFC-Fed Mice Attenuates Hepatic Inflammation

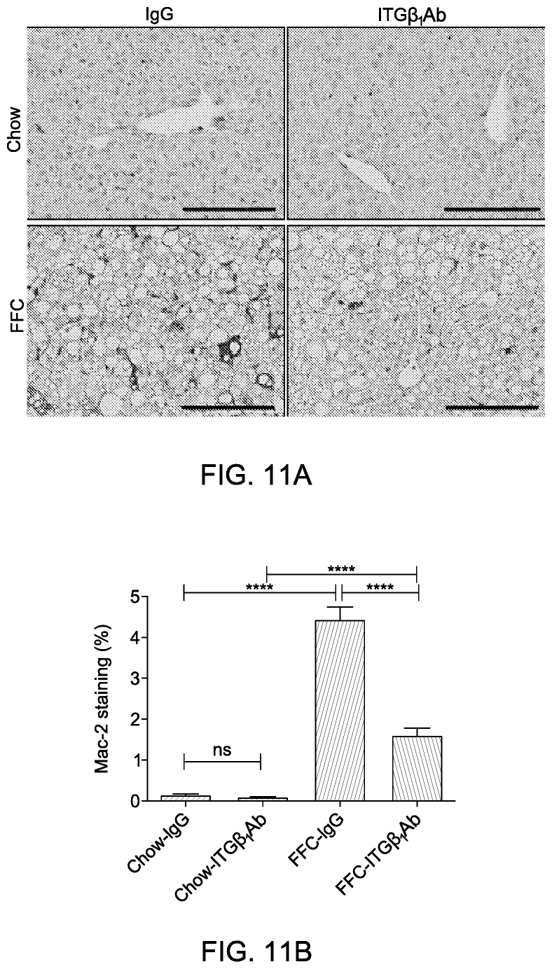

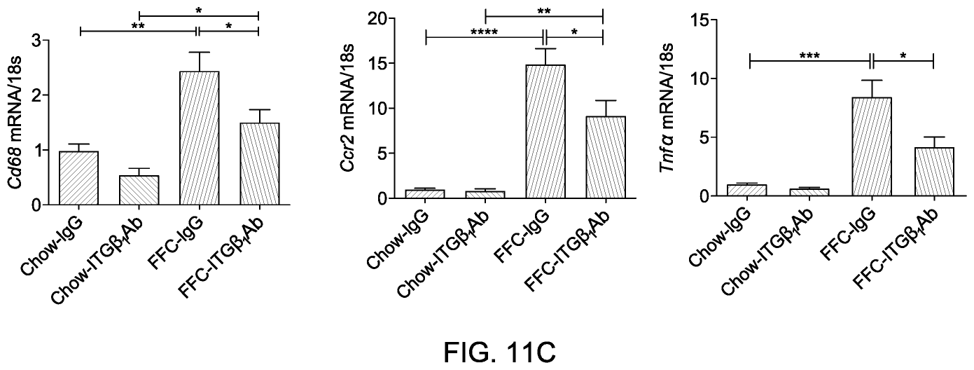

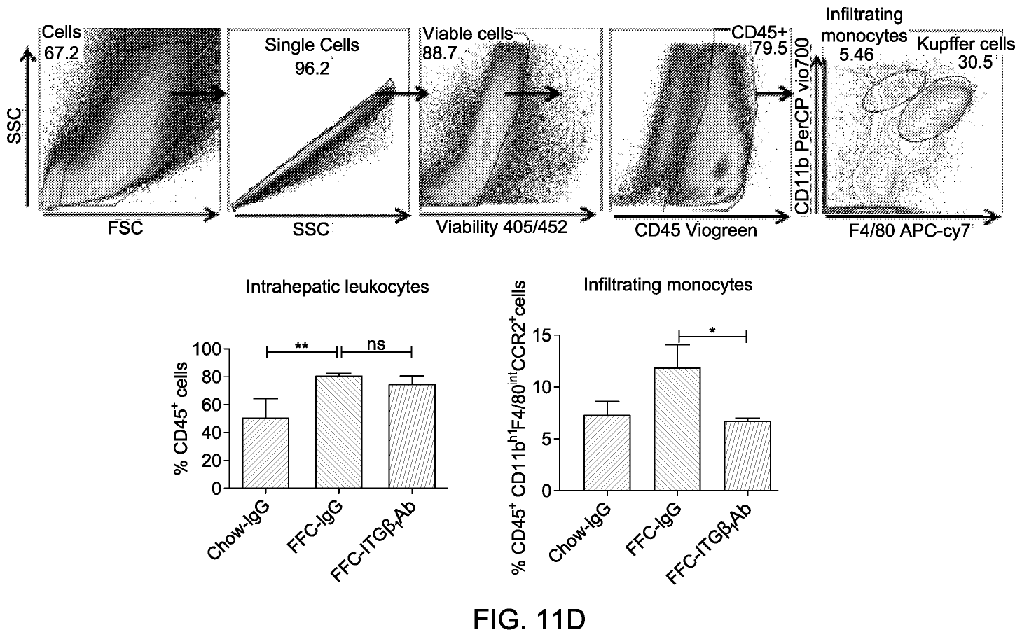

Given the key role of ITGβ₁ in monocyte adhesion to LSECs (FIG. 5D-F), whether ITGβ₁Ab reduces hepatic proinflammatory monocyte recruitment and macrophage-mediated liver inflammation was examined in a dietary mouse model of NASH. Immunostaining of liver tissues revealed that ITGβ₁Ab-treated mice had reduced area positive for Mac-2, a marker of phagocytically active macrophages (FIG. 11A-B). This finding was supported by the decrease in hepatic mRNA expressions of the macrophage marker Cd68, the infiltrating proinflammatory monocyte marker Ccr2, proinflammatory cytokines Tnf-α and Il12b in FFC-fed ITGβ₁Ab-treated mice (FIG. 11C, and FIG. 10B). Furthermore, flow cytometric analysis of the IHL population identified an increase in CD45⁺ cells in the FFC-fed mice, without significant alteration with ITGβ₁Ab treatment (FIG. 11D). In contrast, ITGβ₁Ab-treated FFC-fed mice did display a significant decrease in the infiltrating proinflammatory monocytes (M1 polarized) defined as CD45⁺CD11b^{hi} F4/80^{int} CCR2⁺ cells. Collectively, these findings suggest that blockade of ITGβ₁ reduces hepatic proinflammatory monocyte infiltration and MoMF-mediated liver inflammation.

### Anti-ITGβ₁ Antibody Treatment in FFC-Fed Mice Reduces the Pro-Inflammatory Monocyte Hepatic Infiltration

Macrophages are characterized using a variety of criteria, including ontogeny (yolk sac- vs. bone marrow-derived), and function (pro-inflammatory vs. restorative). Liver macrophages are also frequently classified as resident macrophages (Kupffer cells) or recruited macrophages (i.e., circulating bone marrow-derived monocytes differentiating into macrophages). Functionally, macrophages exist as a continuum, with tissue damaging or pro-inflammatory at one end of the spectrum (M1-like), and restorative macrophages involved in tissue repair and healing at the other end (M2-like).

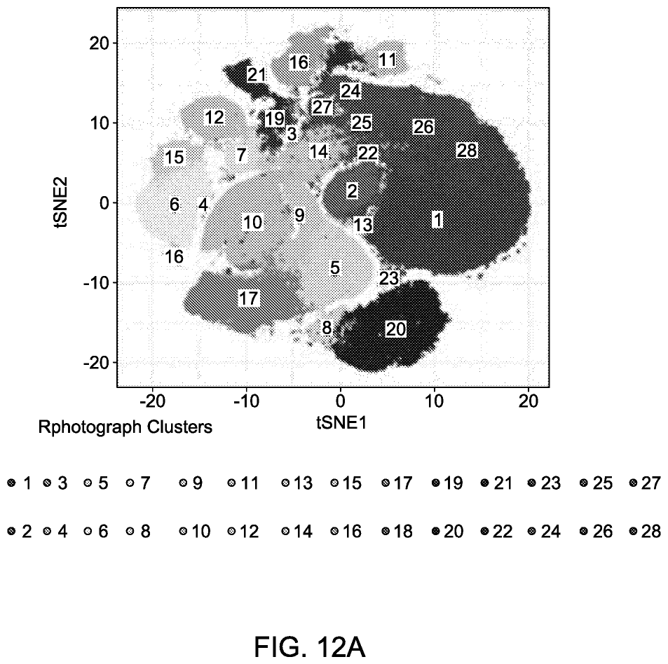

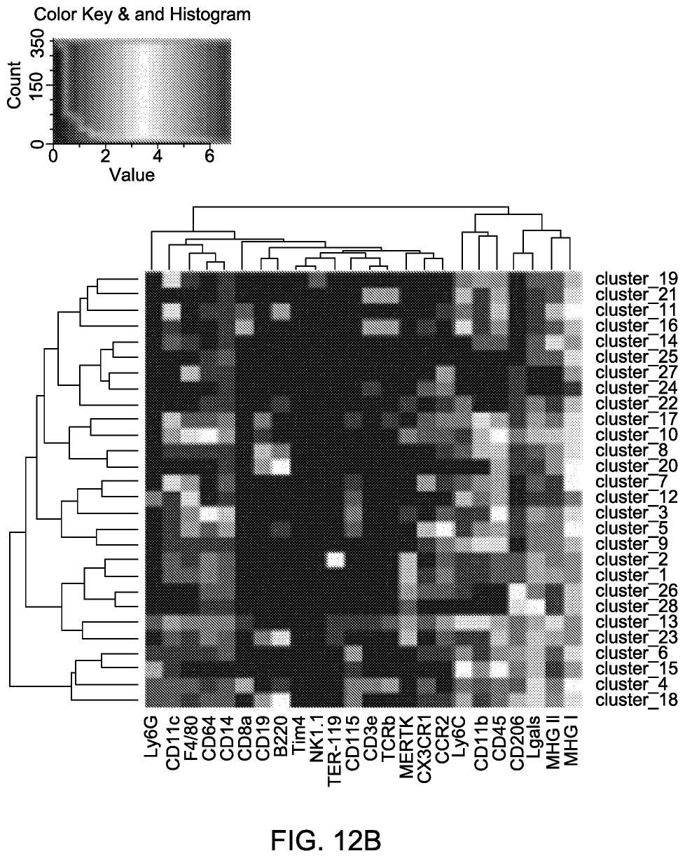

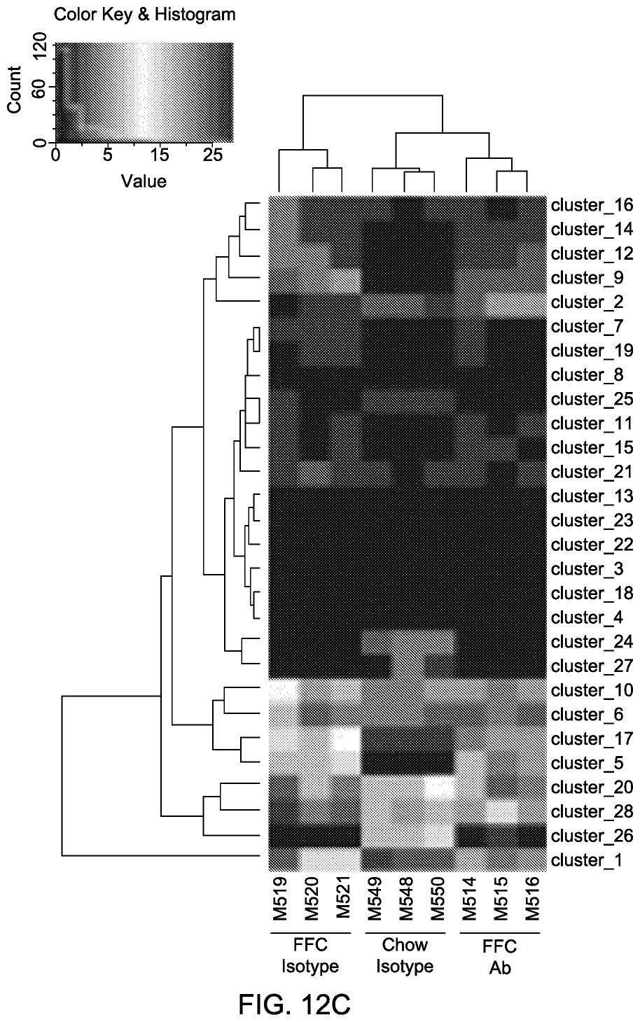

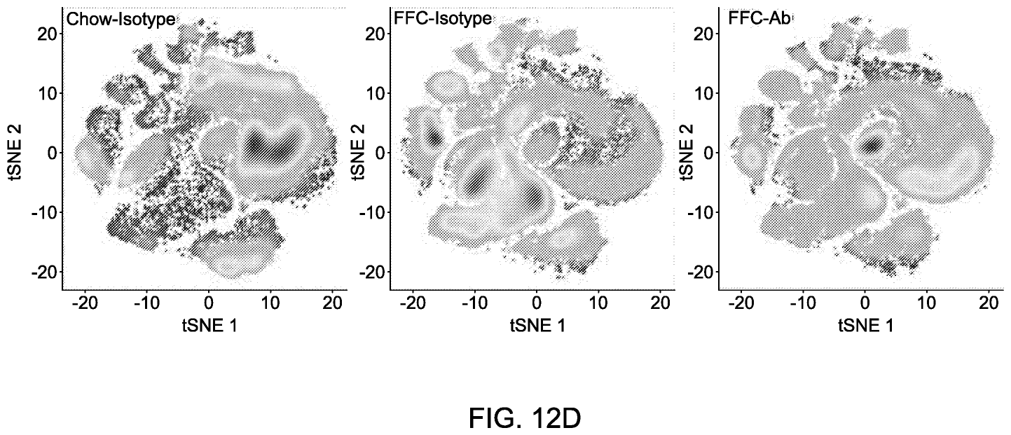

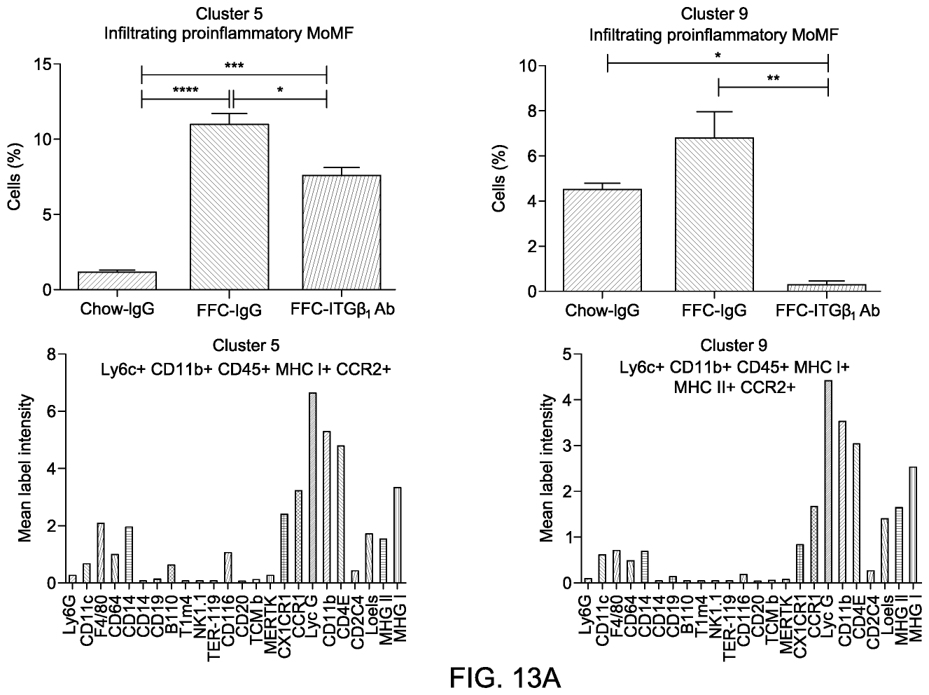

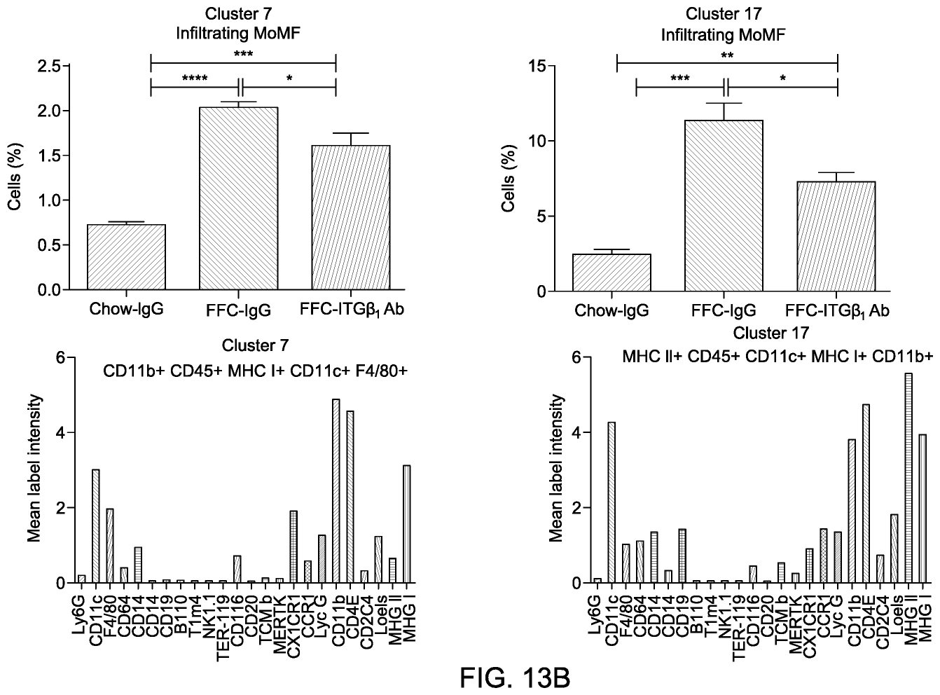

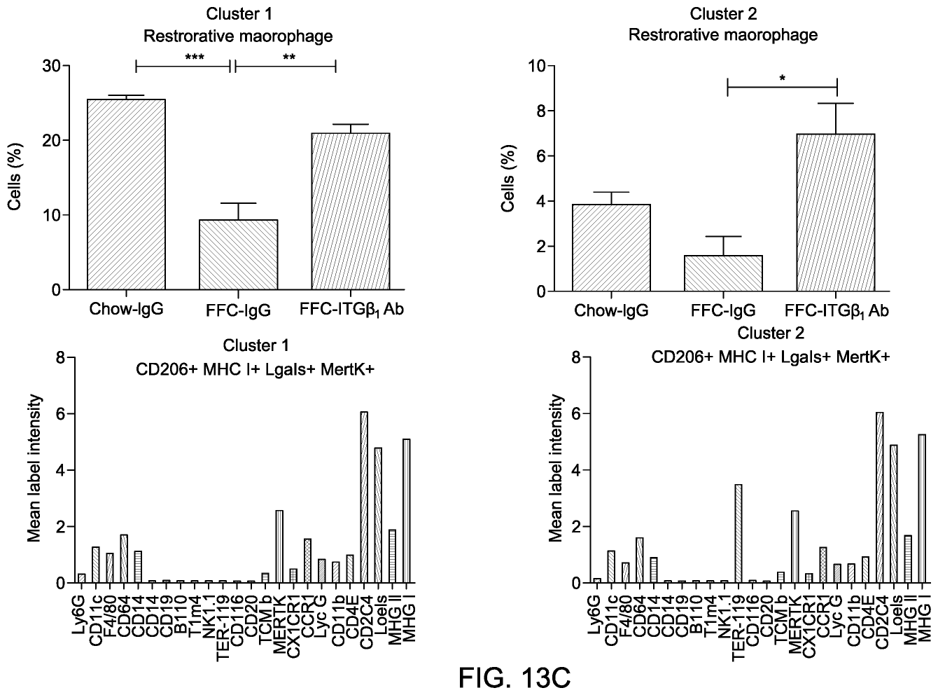

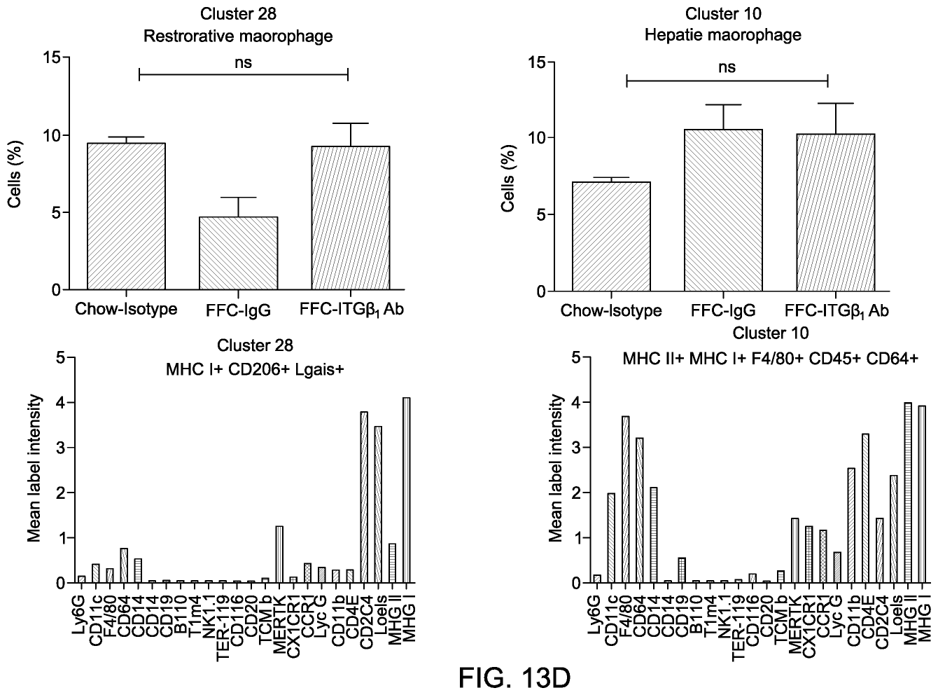

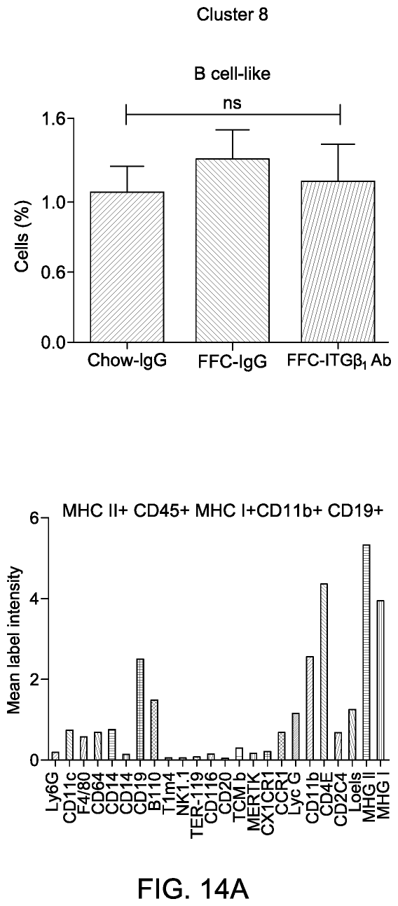

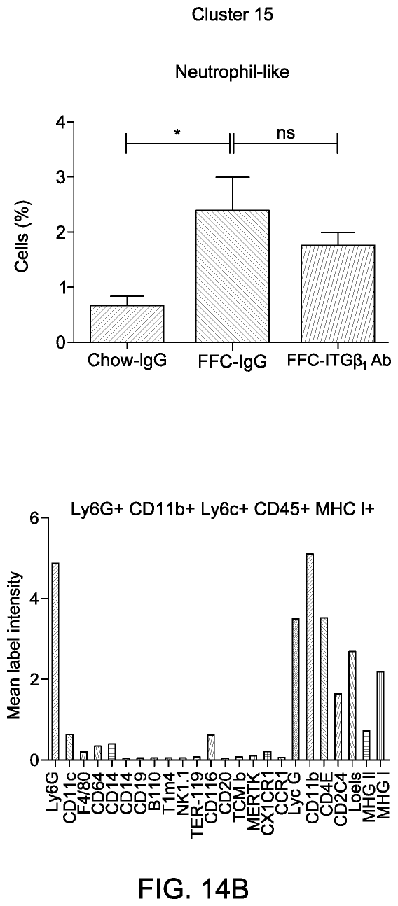

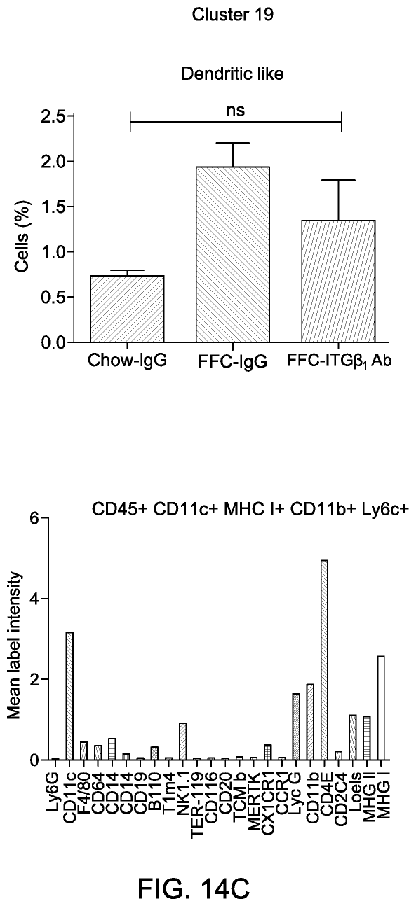

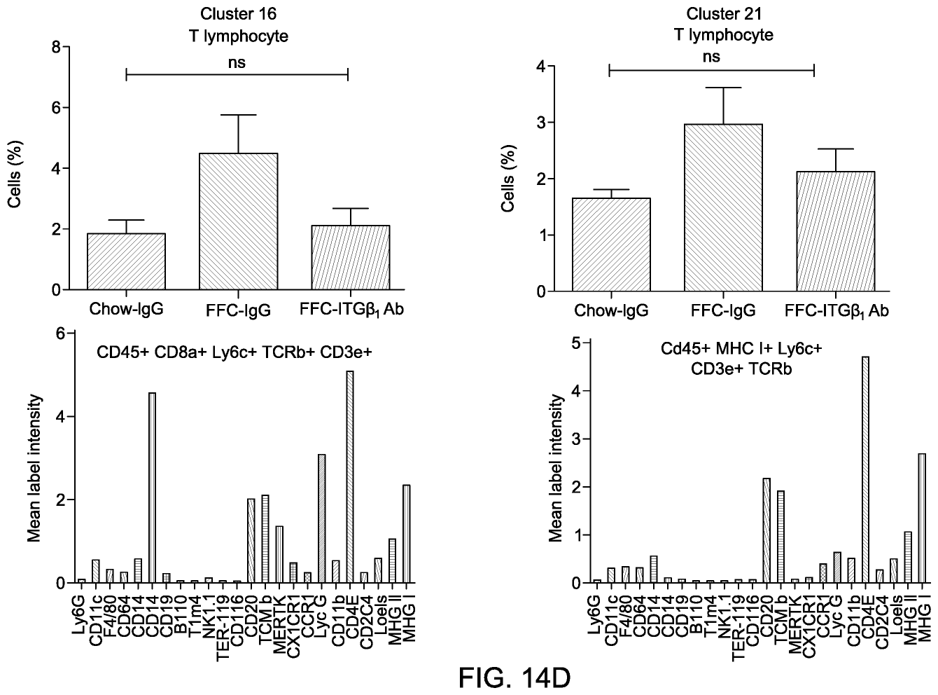

While MoMFs play a crucial role in NASH pathogenesis and progression, various other immune cells including neutrophils, dendritic cells, and lymphocytes are involved in NASH pathogenesis. To determine the contribution of the different subset of macrophages, and other immune cells in ITGβ₁Ab protective effect in NASH, B lymphocytes, T lymphocytes, natural killer cells, NKT cells, dendritic cells and neutrophils in addition to monocytes and macrophages were profiled using the state of the art technology mass cytometry by time-of-flight (CyTOF). Twenty eight clusters were obtained (FIG. 12A) based on the intensities of 24 different cell surface markers (FIG. 12B). Each group of mice had a characteristic pattern of cluster abundance (FIGS. 12C and D). Out of 28 clusters obtained by CyTOF, 13 clusters were differentially expressed between the study groups and categorized into distinct leukocyte subpopulations based on the intensities of individual cell surface markers (Table 6). Clusters 5 and 9 had typical expression markers of infiltrating proinflammatory MoMFs, the abundance of these clusters was increased with the FFC-diet, but significantly reduced with ITGβ₁Ab treatment (FIG. 13A), confirming the flow cytometry data. Likewise, clusters 7 and 17 (FIG. 13B) defined as infiltrating MoMF, were reduced in the FFC-fed ITGβ₁Ab-treated mice. In contrast, clusters 1 and 2 had typical marker expression patterns of alternative, M2 polarized, or restorative macrophages defined by increased expression of the anti-inflammatory surface marker CD206, as well as the hepatic macrophage markers Lgals, MERTK, and F4/80. The abundance of clusters 1 and 2 was decreased in the NASH liver (FFC-fed) mice, but significantly increased with ITGβ₁ blockade (FIG. 13C). Other clusters were assessed and were defined as B cell-like, cluster 8 (FIG. 14A), neutrophil-like, cluster 15 (FIG. 14B), dendritic cell-like, cluster 19 (FIG. 14C), and T lymphocyte cluster 16 and 21 (FIG. 14D). These clusters showed no statistically significant difference between the FFC-fed experimental groups, indicating that the protective effect of ITGβ₁ blockade in the FFC-diet induced NASH is mainly through reduced proinflammatory monocyte trafficking and retention in the liver without significant effect on other immune cells.

TABLE 6
Predominant immune cell markers for the differentially
expressed clusters between the study groups.

| Cluster | Phenotype | Predominant Cell Type |
|---|---|---|
| 5 | Ly6c+ CD11b+ CD45+ MHC I+ CCR2+ | Infiltrating proinflammatory MoMF |
| 9 | Ly6c+ CD11b+ CD45+ MHC I+ MHC II+ CCR2+ | Infiltrating proinflammatory MoMF |
| 7 | CD11b+ CD45+ MHC I+ CD11c+ F4/80+ | Infiltrating MoMF |
| 17 | MHC II+ CD45+ CD11c+ MHC I+ CD11b+ | Infiltrating MoMF |
| 1 | CD206+ MHC I+ Lgals+ MertK+ | Restorative macrophage |
| 2 | CD206+ MHC I+ Lgals+ MertK+ | Restorative macrophage |
| 28 | MHC I+ CD206+ Lgals+ | Restorative macrophage |
| 10 | MHC II+ MHC I+ F4/80+ CD45+ CD64+ | Resident macrophage |
| 8 | MHC II+ CD45+ MHC I+ CD11b+ CD19+ | B cell-like |
| 15 | Ly6G+ CD11b+ Ly6c+ CD45+ MHC I+ | Neutrophil-like |
| 19 | CD45+ CD11c+ MHC I+ CD11b+ Ly6c+ | Dendritic cell-like |
| 16 | CD45+ CD8a+ Ly6c+ TCRb+ CD3e+ | T lymphocyte |
| 21 | CD45+ MHC I+ Ly6c+ CD3e+ TCRb+ | T lymphocyte |

Out of 28 clusters obtained by CyTOF, 13 clusters were differentially expressed between the study groups and categorized into distinct leukocyte subpopulations based on the intensities of individual cell surface markers.

### Anti-ITGβ₁ Antibody Treatment Reduces FFC Diet-Induced Liver Injury and Fibrosis in Murine NASH

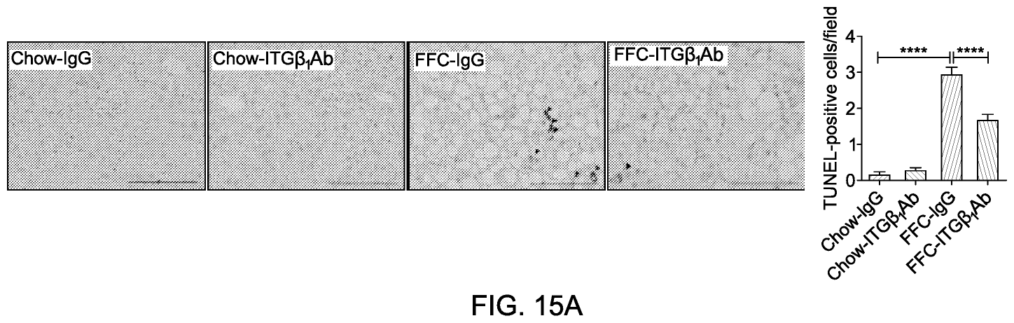

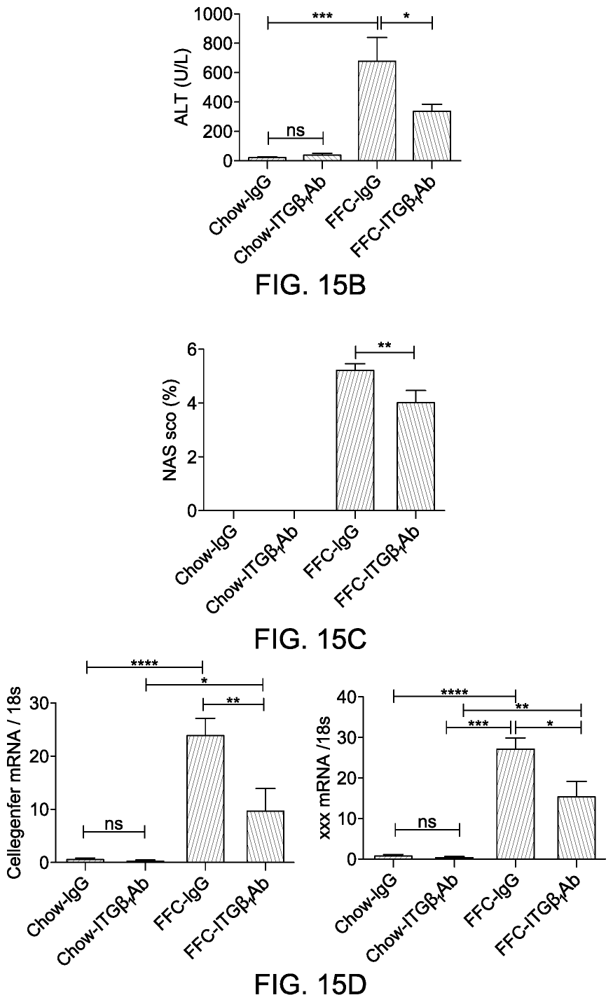

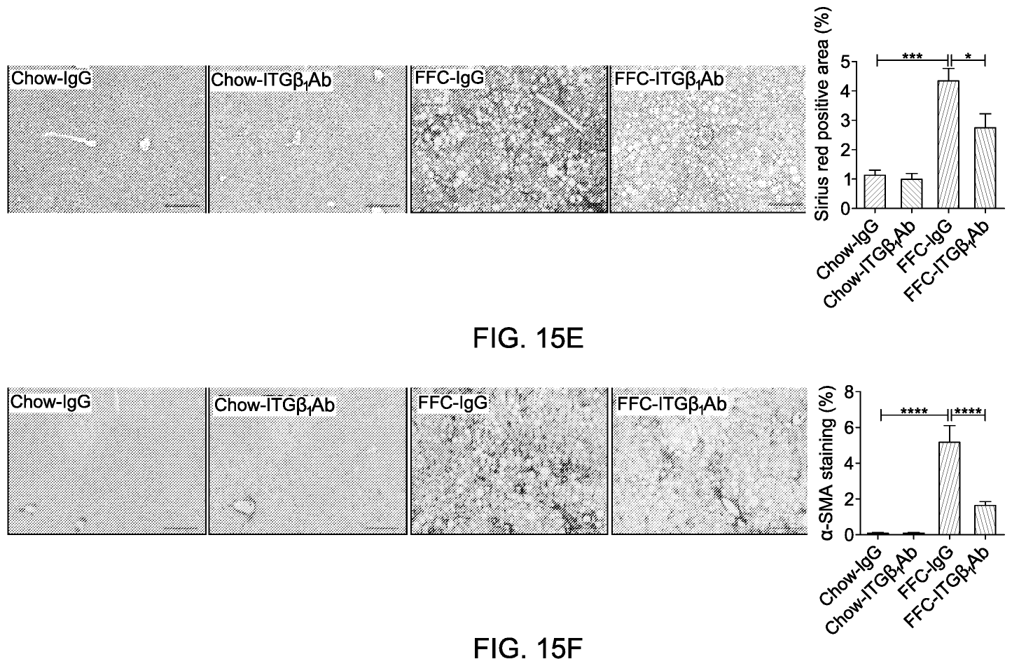

To determine if reduced hepatic inflammation through ITGβ₁ blockade may protect against NASH progression and liver fibrosis, liver injury and fibrosis were examined. FFC-fed, ITGβ₁Ab-treated mice were relatively protected against hepatocyte apoptosis compared to control IgG-treated mice, as demonstrated by reduced TUNEL-positive cells (FIG. 15A) and serum alanine aminotransferase (ALT) levels (FIG. 15B) as well as reduced NAFLD activity score (NAS) (FIG. 15C) when compared to IgG-treated mice on the same diet. Next, the expression of fibrosis-related genes were examined, mRNA levels of both Collagen 1α1 and Osteopontin were elevated in the FFC-fed mice, and significantly decreased with ITGβ₁Ab treatment (FIG. 15D), indicating the possible anti-fibrotic effect of ITGβ₁Ab. This finding was further confirmed by Sirius red staining (FIG. 15E) as well as α-smooth muscle actin (α-SMA) immunohistochemistry (FIG. 15F). Taken together, these findings indicate a protective effect of ITGβ₁Ab against NASH-associated liver injury and fibrosis in diet-induced NASH.

Taken together, these results demonstrate that ITGβ₁ neutralizing antibody can reduce pro-inflammatory monocyte hepatic infiltration and expand the restorative macrophage population, and that blocking ITGβ₁ can attenuate liver injury, inflammation and fibrosis.

### Example 2: Anti-VCAM-1 Antibody Treatment Reduces FFC Diet-Induced Liver Fibrosis in Murine NASH

### Materials and Methods

### Materials

Palmitate (PA) (P0500) was obtained from Sigma-Aldrich (St. Louis, Mo.). AGI-1067 (216167-82-7) was from MedChemExpress (Princeton, N.J.). URMC-099 was kindly provided by Dr. Harris A. Gelbard (University of Rochester Medical Center, Rochester, N.Y.). SB203580 (559389) was from Millipore Sigma (St. Louis, Mo., USA). Primary antisera employed for the studies include: anti-alpha smooth muscle actin (α-SMA) (ab124964), and anti-human VCAM-1 (ab134047) antibody from Abcam (Cambridge, Mass.), anti-p-p38 (9211), anti-p38 (9212), anti-p-MKK3/MKK6 (12280), anti-MKK3 (8535), p-JNK (9255), JNK (9252), and mouse VCAM-1 (32653) from Cell Signaling Technology (Danvers, Mass.), anti-GAPDH (MAB374) from Millipore Sigma, anti-β-actin (sc-47778) from Santa Cruz Biotechnologies (Santa Cruz, Calif.), and anti-Galectin-3 (14530181) from Thermo Fisher Scientific (Waltham, Mass.). Neutralizing anti-VCAM-1 antibody (GTX14360) and IgG isotype control (BE0088) were obtained from GeneTex and InVivoMab, respectively.

### Human Liver Samples

De-identified archived liver specimens obtained by liver biopsy or surgical hepatic resection from patients with normal liver, isolated steatosis, or NASH were acquired through a human study. Histological diagnosis was based on established NASH criteria as assessed by experienced pathologist. Subjects with other chronic liver diseases (cholestatic liver disease, hemochromatosis, excessive alcohol consumption, viral hepatitis, Wilson disease, drug-induced liver disease, and alpha-1-antitrypsin deficiency) were excluded.

### Cells

Transformed mouse liver sinusoidal endothelial cells (TSEC) were obtained. Primary human liver sinusoidal endothelial cells (LSEC) were purchased from ScienCell Research Laboratories (San Diego, Calif.). To yield primary mouse LSECs, cell suspensions obtained with liver collagenase perfusion were centrifuged at 50 g for 2 minutes to remove hepatocytes. The supernatant which includes non-parenchymal cells was subjected to LSEC isolation using CD146 MicroBeads (Miltenyi Biotec, Bergisch Gladbach, Germany) following the manufacture's instruction. TSEC, primary human LSEC, and primary mouse LSEC were cultured in Endothelial Cell Growth Medium (ECM, ScienCell Research Laboratories) consisting of 5% FBS, 1% endothelial cells growth supplement, and 1% primocin (InVivoGen, San Diego, Calif.) solution. All the cell cultures were maintained at 37° C. in a humidified atmosphere of 5% CO2.

### PA Treatment of Cells

PA was dissolved in isopropanol at a concentration of 80 mM as a stock solution. The PA stock solution was added to Endothelial Cell Growth Medium containing 1% fatty acid free low endotoxin BSA; the final experimental concentrations of PA 500 or 800 μM were used for the treatment of cells. For the negative control non-treated (vehicle-treated) cells, the same concentration of isopropanol was added to Endothelial Cell Growth Medium containing 1% BSA.

### Immunoblot Analysis

Cells were lysed using RIPA buffer (50 mM Tris-HCl, pH 7.4; 1% Nonidet P-40; 0.25% sodium deoxycholate; 150 mM NaCl; 1 mM EDTA with protease inhibitors) followed by centrifugation at 15,000 g for 15 minutes at 4° C. Protein concentrations of the lysates were measured by the Bradford assay method (Sigma-Aldrich). Equal amount of protein were loaded onto Sodium dodecyl sulfate (SDS)-Polyacrylamide gel electrophoresis (PAGE) gels, transferred to nitrocellulose membrane (Bio-Rad, Hercules, Calif.) and incubated overnight with the primary antibody of interest. All primary antibodies were used at a dilution of 1:1,000 unless otherwise recommended by the manufacturer. Horseradish peroxidase-conjugated secondary antibodies against rabbit (Alpha Diagnostic International, San Antonio, Tex.) or mouse (Southern Biotech, Birmingham, Ala.) were used at a dilution of 1:5,000 and incubated for 1 hour at room temperature. Proteins were detected using enhanced chemiluminescence reagents (GE Healthcare, Chicago, Ill.). β-actin and GAPDH protein levels were used as loading controls.

### Animals

C57BL/6J male mice were purchased from Jackson Laboratory (Bar Harbor, Me.). Mice were housed and bred in a temperature-controlled 12:12-hour light-dark cycle facility with free access to diet. The mice were fed either a chow diet (5053 PicoLab Rodent Diet 20, LabDiet, St Louis, Mo.) or a diet rich in fat, fructose, and cholesterol (FFC) at 8-weeks old for 24 weeks. FFC diet consists of 40% energy as fat (12% saturated fatty acid, 0.2% cholesterol) (AIN-76A Western Diet, TestDiet, St Louis, Mo.), with fructose (23.1 g/L) and glucose (18.9 g/L) in the drinking water. The FFC diet induces steatohepatitis with pronounced hepatocellular ballooning, lipoapoptosis, and progressive fibrosis with a high fidelity to the human NASH histology and metabolic profile. At 20 weeks on the diet, the mice were randomized to receive either anti-VCAM-1 neutralizing antibody (M/K-2.7), (Genetex, GTX14360) or IgG isotype antibody (BE0088, InVivoMAb). Mice were injected with 10 mg/kg body weight of either of the antibodies intraperitoneally, twice per week for 4 weeks. Another cohort of Chow or FFC diet-fed mice was randomized to receive either vehicle or VCAM-1 inhibitor succinobucol (AGI-1067). Mice were injected with either vehicle or 25 mg/kg body weight of AGI-1067 intraperitoneally, daily for 15 days. Total caloric intake at the 3rd week of VCAM-1 antibody treatment and the first week of AGI-1067 treatment was calculated based on the weight of food and drinking water consumption. At the third week of anti-VCAM-1 neutralizing antibody treatment, metabolic parameters, including oxygen consumption, carbon dioxide production, and locomotor activity, were measured using a Comprehensive Lab Animal Monitoring System (CLAMS, Columbus Instruments, OH). Anti-VCAM-1 antibody from Bio Xcell (West Lebanon, N.H.), (BE0027) was used for the CLAMS study. Blood glucose levels and plasma insulin levels were measured using Assure 4 (Arkray, Edina, Minn.) and Ultra-Sensitive Mouse Insulin enzyme-linked immunosorbent assay (ELISA) kit (Crystal Chem Inc., Downers Grove, Ill.), respectively. Homeostasis model assessment of insulin resistance (HOMA-IR) was calculated by using the following formula: HOMA-IR=26×fasting insulin level (ng/mL)×fasting glucose level (mg/dL)/405. Mice were sacrificed under general anesthesia induced by a ketamine/xylazine cocktail (83 mg/kg ketamine, 16 mg/kg xylazine, intraperitoneal injection). All interventions were made during the light cycle. Blood and liver samples were collected for further study.

### RNA Sequencing and Bioinformatics Analysis

RNA sequencing was performed on whole liver from both Chow and FFC diet-fed mice. Three mice per group were included for the study. RNA libraries were prepared using 200 ng of total RNA according to manufacturer's instructions for the TruSeq RNA Sample Prep Kit v2 (Illumina, San Diego, Calif.). The concentration and size distribution of the completed libraries was determined using an Agilent Bioanalyzer DNA 1000 chip (Santa Clara, Calif.) and Qubit fluorometry (Invitrogen, Carlsbad, Calif.). Libraries were sequenced at 53 million to 90 million reads per sample following Illumina's standard protocol using the Illumina cBot and HiSeq 3000/4000 PE Cluster Kit. The flow cells were sequenced as 100×2 paired end reads on an Illumina HiSeq 4000 using HiSeq 3000/4000 sequencing kit and HCS v3.3.20 collection software. Base calling was performed using Illumina's RTA version 2.5.2. Genes with Log 2 fold change (Log FC) more than 1.5 and p-value less than 0.05 were considered differentially expressed. Ingenuity Pathway Analysis (IPA) software was used to analyze the data.

### Liver Triglyceride and Alanine Aminotransferase Measurement

Liver triglyceride levels were measured in mouse liver homogenates. Fifty milligrams of liver tissue was homogenized in a 5% NP-40 solution. EnzyChrom Triglyceride Kit (BioAssay System, CA) was used for the assay according to the manufacturer's instruction. Photometric absorbance was read at 570 nm using a Synergy H1 microplate reader (BioTek). Serum alanine aminotransferase (ALT) levels were measured by VetScan2 (Abaxis Veterinary Diagnostics, Union City, Calif.).

### Histology, Immunohistochemistry, and Digital Image Analysis

Liver histology was performed using tissue fixed in 10% formalin, dehydrated, and embedded in paraffin. Hematoxylin and eosin (H&E) staining and Sirius red staining were performed. Severity of NASH was assessed using nonalcoholic fatty liver disease (NAFLD) activity score (NAS), a semi-quantitative score that accounts for steatosis, ballooned hepatocytes, and lobular inflammation. Terminal deoxynucleotidyl transferase deoxyuridine triphosphate nick-end labeling (TUNEL) assay was performed using the ApopTag Peroxidase In Situ Apoptosis Detection Kit (Millipore Sigma, St. Louis, Mo.); diaminobenzidine (DAB) was used as a peroxidase substrate (Vector Laboratories, Burlingame, Calif.); 0.5% methyl green was used for the counterstain. For the immunohistochemistry studies, formalin-fixed paraffin-embedded liver tissue sections were deparaffinized, hydrated, and stained with antibody against VCAM-1 (1:200), Galectin-3 (1:250) or alpha smooth muscle actin (α-SMA) (1:1000) for mouse tissues, and VCAM-1 (1:500) for human tissues. Bound antibodies derived from mouse or rabbit were detected using a Vectastain ABC kit (Vector Laboratories) or EnVision System HRP (Dako), respectively, and DAB substrate (Vector Laboratories) according to the manufacturer's instructions; the tissue sections were counterstained with hematoxylin. Sirius red-positive, VCAM-1, Galectin-3, or α-SMA-positive areas were quantified by digital image analysis of 10 random fields per slide per animal using the ImageJ software. TUNEL-positive cells were quantified by counting positive nuclei in 10 random fields per slide per animal.

### Quantitative Real-Time PCR

Total RNA was isolated with the RNeasy Mini Kit (Qiagen, Valencia, Calif.) and was reverse transcribed with moloney murine leukemia virus reverse transcriptase and oligo-dT random primers (both from Invitrogen, CA, USA). Quantification of gene expression was performed by real-time PCR using SYBR green fluorescence on a LightCycler 480 instrument (Roche Applied, IN, USA). Target gene expression was calculated using the ΔΔCt method and was normalized to 18s rRNA expression levels, which were stable across experimental groups.

### Mass Cytometry by Time-of-Flight (CyTOF) Analysis

Intrahepatic mouse leukocytes were isolated using liver dissociation kit and Percoll gradient centrifugation. Cells were suspended in Maxpar Cell Staining Buffer (CSB, Fluidigm, San Francisco, Calif.) and labelled with 0.5 μM cisplatin (Fluidigm) solution. After centrifugation, cells were resuspended in CSB prior to addition of the antibody cocktail (composition shown in Table 5) in an equal volume of CSB. Cells were incubated with gentle agitation at room temperature for 45 minutes. Following wash, cells were fixed overnight with 2% paraformaldehyde with gentle agitation at 4° C. DNA intercalation was performed by adding 1:10000 diluted 125 μM of Cell-ID™ Intercalator-Ir (Fluidigm) with gentle agitation in 4° C. for 30 minutes. Cells were resuspended in 1:10 dilution of EQ beads (EQ Four Element Calibration Beads, Fluidigm), and then loaded onto Helios sample loader for data acquisition. Mass cytometry was performed in the Immune Monitoring Core at Mayo Clinic, and employed antibodies conjugated to stable heavy-metal isotopes to detect cellular antigens by mass cytometry time-of-flight (CyTOF) and enables comprehensive profiling of the phenotype and function of the intrahepatic leukocytes. After data acquisition, fcs files were normalized using CyTOF Software (version 6.7.1014). Cleanup of cell debris, removal of doublets and dead cells was performed using FlowJo software version 10.5.3 (Ashland, Oreg.). Cleaned fcs files were analyzed by the R-based tool Cytofkit version 3.8. Clustering and dimensionality reduction to 20,000 events per file was performed using the Rphenograph algorithm that included all 30 markers in the panel (Table 5). Visualization of clusters was mapped onto a tSNE map. Relative marker intensities and cluster abundances per sample were visualized by heatmap.

### B Cell Purification and Cell Viability Assay

B cells were obtained by magnetic isolation from mouse spleen according to manufacturer's instruction. In brief, mouse spleen was dissociated using Spleen Dissociation Kit (Miltenyi, 130-095-926), cell suspension was subjected to B cell purification using negative selection with Dynabeads mouse CD43 (Invitrogen, 11422D). Flow cytometric analysis confirmed that the purity of B cells was >98% as assessed by the ratio of CD45+ CD19+ cells out of whole isolated cells. Cells were grown in 96-well flat bottom plate (5×10⁵ cells per well). Cell viability was assessed using CellTiter-Glo luminescent cell viability assay (Promega, Madison, Wis.) (G7570) after 4 hour treatment with either vehicle or 20 μM of lysophosphatidylcholine (LPC), a toxic lipid mediator.

### Statistical Analysis

Data are expressed as the means±SEM. Differences between multiple groups were compared using one-way analysis of variance followed by Bonferroni's multiple comparisons test or Student t test when comparing 2 groups. *, **, ***, **** indicate statistical significance with p<0.05, p<0.01, p<0.001 and p<0.0001 respectively. Statistically non-significant results were labelled as ns where appropriate. All analyses were performed using GraphPad Prism 8 software (CA, USA).

### Results

### Adhesion Molecule VCAM-1 is Upregulated in Murine NASH

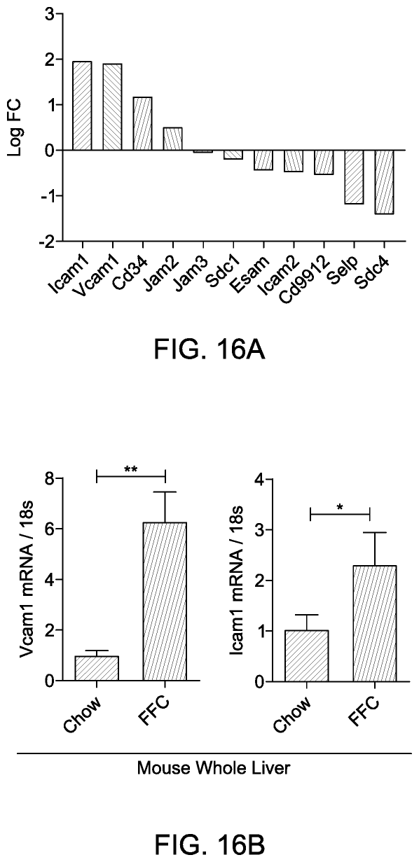

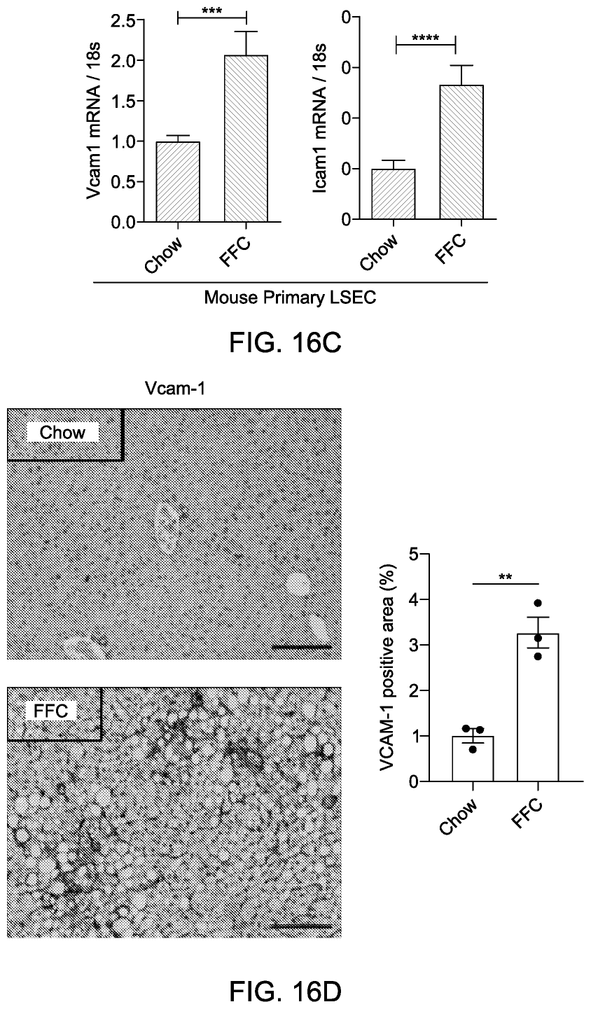

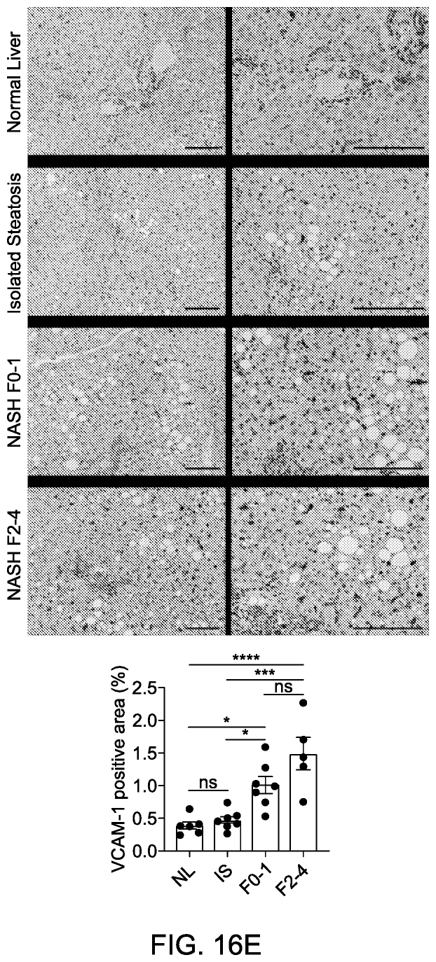

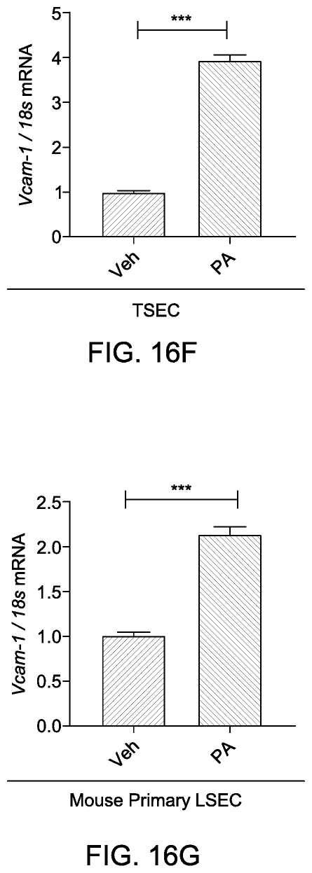

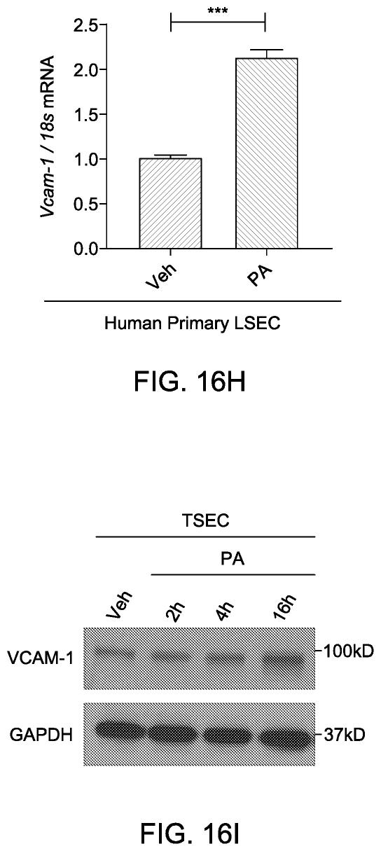

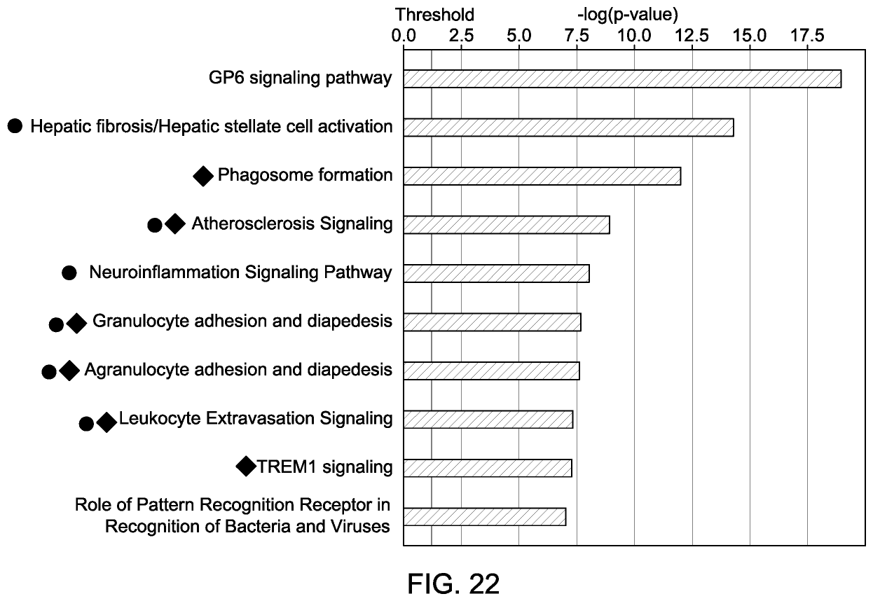

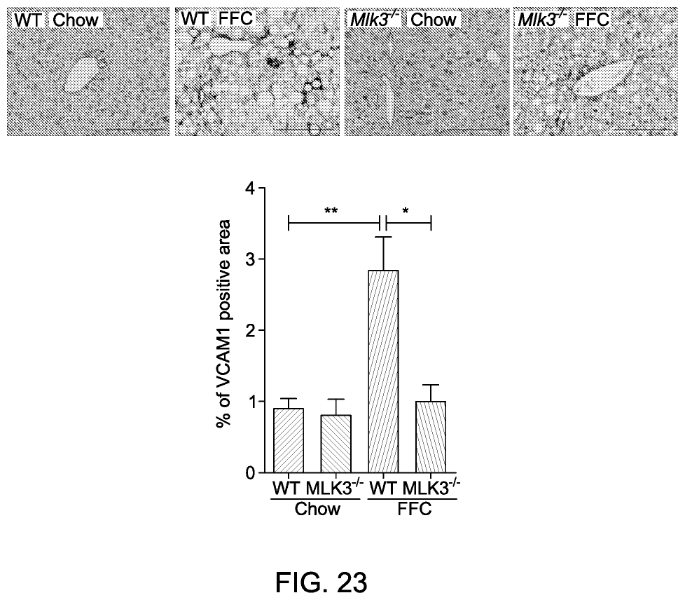

To gain a comprehensive insight into the pathophysiology of NASH, unbiased transcriptomic analysis using RNA sequencing (RNA-seq) was performed on whole livers from a diet-induced NASH mouse model; mice were fed either the FFC diet, or normal chow-fed control mice. Out of 13733 coding transcripts detected in the RNA-seq, 986 genes were differentially upregulated in FFC mice livers. These upregulated gene sets were subjected to Ingenuity Pathway Analysis (IPA), and it was found that among the top 10 ranked over-represented canonical pathways, 6 pathways were profoundly involved in leukocyte adhesion and differentiation (FIG. 22), suggesting a strong pathophysiological impact of leukocyte adhesion to LSEC on the inflammatory response in NASH. To identify the molecules implicated in leukocyte adhesion to LSEC, “candidate genes” which encode adhesion molecules expressed in endothelial cells and act as binding partners of leukocyte adhesion molecules such as integrins were examined. Among these candidate genes, only Icam1 and Vcam1, which encode intercellular adhesion molecule-1 (ICAM-1) and VCAM-1, respectively, were differentially upregulated in NASH liver (FIG. 16A). Real time qPCR demonstrated that both Vcam1 and Icam1 were significantly upregulated in the whole liver as well as the isolated LSEC of FFC-fed mice compared to Chow-fed mice (FIGS. 16B and C), supporting the findings obtained by the whole transcriptome study. Notably, diet-induced NASH liver had 6 fold as much mRNA for Vcam1 as control mice. Immunohistochemistry confirmed increased VCAM-1 protein expressions in liver sinusoidal endothelium of FFC-fed mice compared to Chow-fed mice (FIG. 16D). The clinical relevance of this finding was conformed in histological section from human livers, indicating that NASH patients had significant increase in hepatic VCAM-1 protein expressions compared to those with normal liver or isolated hepatic steatosis as assessed by immunohistochemistry (FIG. 16E). These findings indicate that in NASH liver, pathways related to leukocyte adhesion are profoundly activated, and the adhesion molecule VCAM-1 is robustly upregulated, implicating VCAM-1 in NASH pathogenesis.

### VCAM-1 is Upregulated in LSEC Under Lipotoxic Conditions Via a MAPK Pathway and an NF-κB Dependent Mechanism

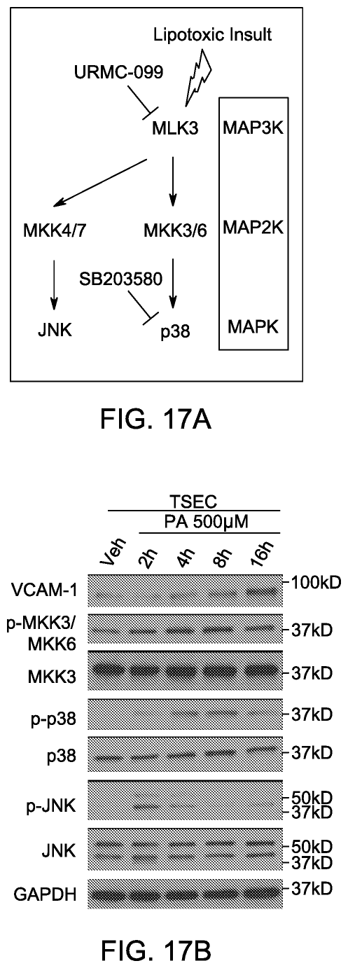

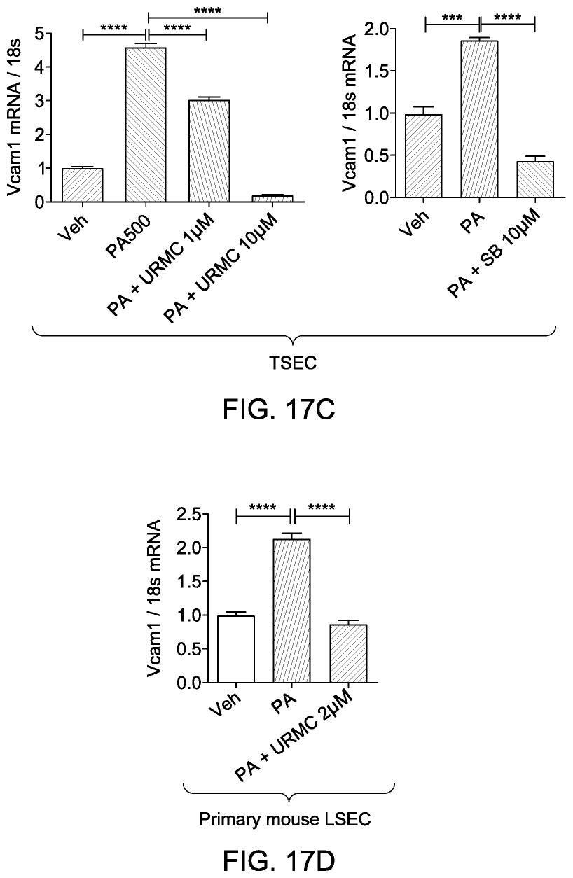

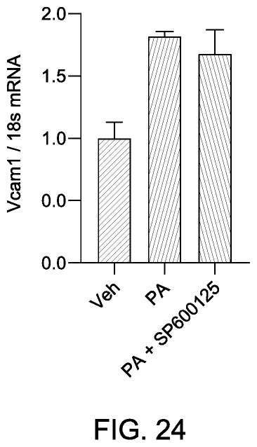

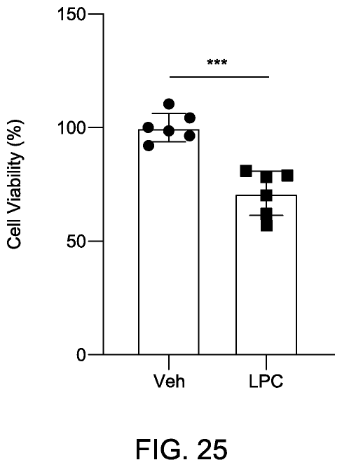

To examine whether VCAM-1 is upregulated in LSEC by lipotoxic stress in vitro, three different types of LSEC, namely transformed sinusoidal endothelial cell line (TSEC), primary LSEC isolated from wild-type mice, and human primary LSEC were used. These cells were treated with palmitate (PA), one of the most common saturated free fatty acids in plasma in NASH patients. It was confirmed that PA treatment increased mRNA expression levels of Vcam-1 in all these cell types (FIG. 16F-H), strongly suggesting that Vcam1 is upregulated in LSEC under lipotoxic conditions. Furthermore, it was demonstrated that in TSEC, VCAM-1 expression was increased also at the protein level during the lipotoxic insult (FIG. 161). The mechanisms underlying VCAM-1 induction in LSEC under lipotoxic stress was next explored. It was first shown that PA treatment in LSEC activated a MAPK signaling cascade in TSEC as assessed by phosphorylation of the MAP2K kinase 3/6 (MMK3/6), the MAPK p38, and c-Jun N-terminal kinase (JNK) (FIGS. 17A and B). Furthermore, pharmacological inhibition of the mitogen activated protein 3 kinase (MAP3K) MLK3, or p38 significantly attenuated PA-induced VCAM-1 mRNA expression in TSEC (FIGS. 17A and C), while JNK inhibition did not alter VCAM1 expression in LSEC under lipotoxic conditions (FIG. 24). Reduction of PA-induced VCAM-1 expression by pharmacological MLK3 inhibition was also observed in mouse primary LSEC (FIG. 17D), suggesting a major role of MAPK signaling pathway in toxic lipid-induced VCAM-1 upregulation in LSEC. Collectively, these findings suggest that lipotoxic stress upregulates VCAM-1 expression in LSEC, via a MLK3-dependent mechanism.

### Neither the Metabolic Phenotype, Nor the Steatosis was Altered by Anti-VCAM-1 Antibody Treatment in FFC Diet-Fed Mice.

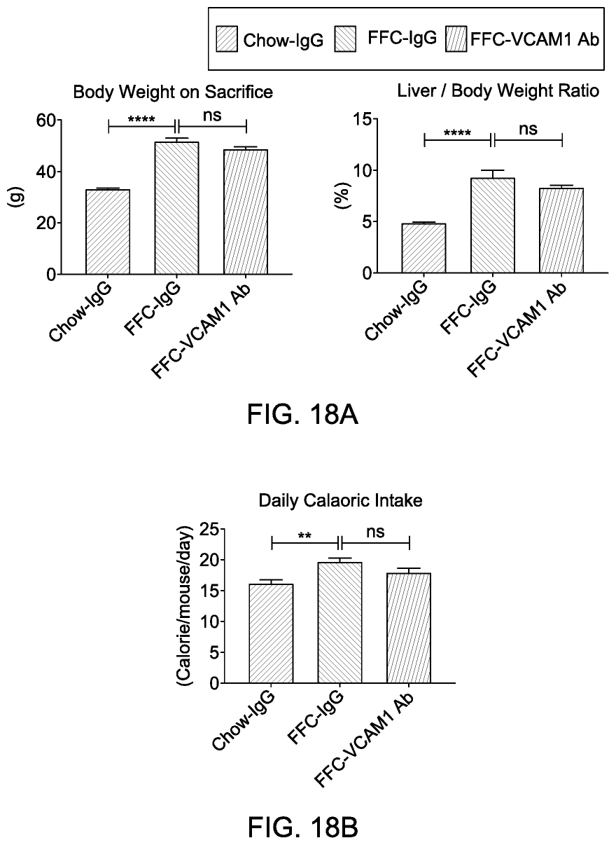

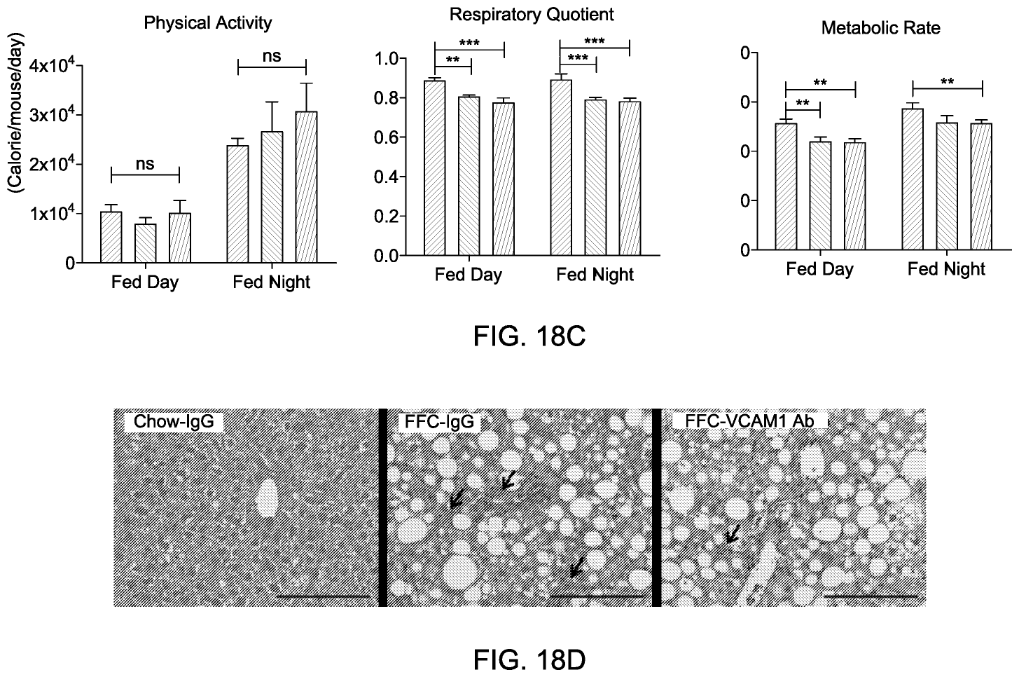

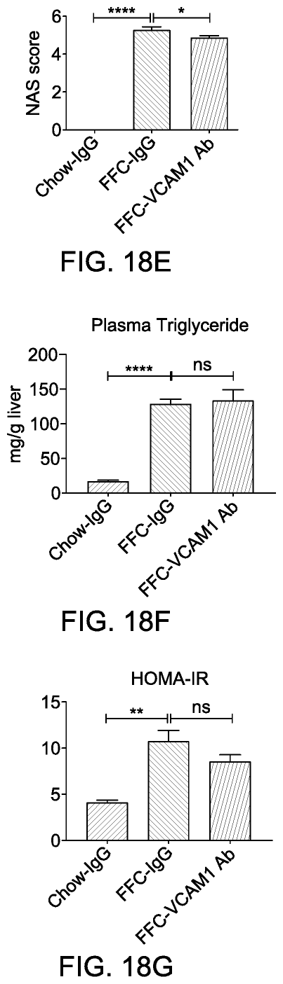

Based on the findings that VCAM-1 is upregulated under lipotoxic conditions both in vitro and in vivo, the therapeutic effect of VCAM-1 neutralizing antibody in the diet-induced murine NASH model was examined. Wild-type mice were fed either chow or the FFC diet for 24 weeks. At 20 weeks of the diet, mice were treated with either anti-VCAM-1 neutralizing antibody (VCAM1 Ab) or control IgG isotype antibody (IgG) twice per week for 4 weeks. Body weight and liver to body weight ratio at the time of sacrifice (FIG. 18A), and total daily caloric intake (FIG. 18B) were significantly increased with the FFC diet, but similar between VCAM1 Ab-treated and control IgG-treated groups. Metabolic phenotyping studies also indicated that physical activity, respiratory quotient, and metabolic rate were also similar between FFC-fed VCAM1 Ab-treated versus control IgG-treated mice (FIG. 18C). Moreover, histological examination of the liver by hematoxylin and eosin (H&E) stain displayed similar extent of steatosis in the FFC-fed mice from the different treatment groups (FIG. 18D). Furthermore, triglyceride content in liver tissue and homeostasis model assessment of insulin resistance (HOMA-IR) were increased with the FFC diet, but were not different between the 2 treatment groups on the FFC diet (FIGS. 18F and G). VCAM1 Ab-treated FFC-fed mice had less inflammatory infiltrates compared to IgG-treated FFC-fed mice (FIG. 18D), resulting in significantly reduced NAS score in the FFC-fed VCAM1 Ab-treated mice when compared to control IgG-treated mice (FIG. 18E). Collectively, these data support that VCAM1 Ab treatment in FFC-fed mice was well tolerated, and did not affect the metabolic phenotype or the hepatic steatosis, but did lower the NAS score appropriately by reducing cellular inflammatory infiltrate.

### Anti-VCAM-1 Antibody Treatment in FFC-Fed Mice Attenuates Hepatic Injury and Inflammation.

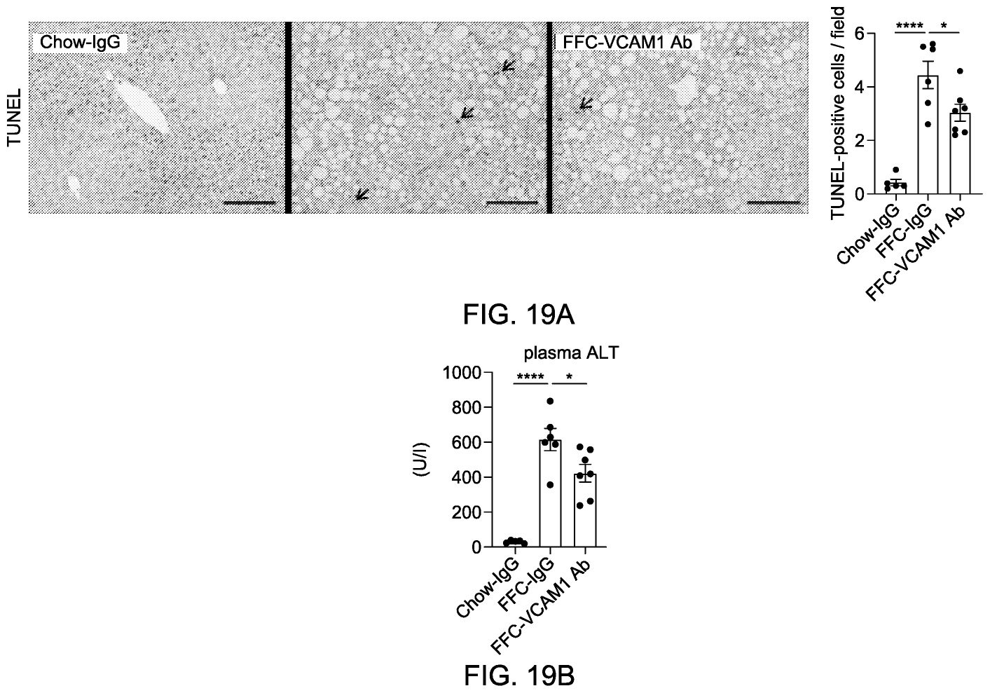

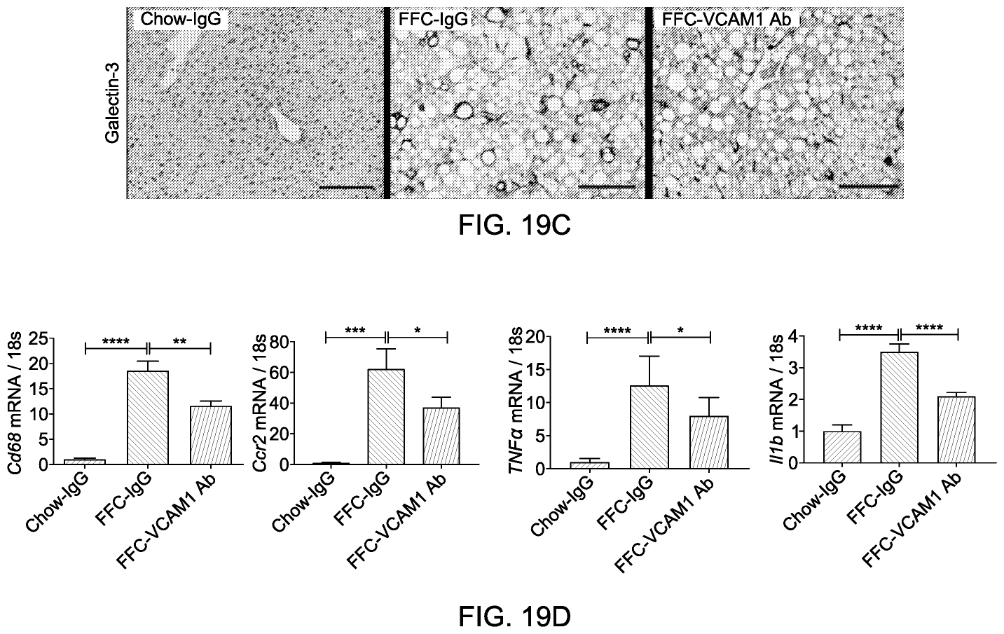

Next, whether VCAM-1 neutralizing antibody reduces liver injury was tested in the dietary mouse model of NASH. FFC-fed, VCAM1 Ab-treated mice showed less TUNEL-positive cells in the liver (FIG. 19A) as well as decreased plasma ALT levels (FIG. 19B) compared to control IgG-treated mice on the same diet, suggesting that VCAM1 Ab treatment attenuates liver injury with reduced apoptotic hepatocytes in NASH. Based on the recent finding that VCAM-1 on LSEC surface mediates monocyte adhesion to LSEC, whether the reduced liver injury in VCAM1 Ab treated mice was associated with attenuated monocyte-derived macrophage (MoMF) hepatic infiltration and inflammation was examined. Immunostaining of liver tissues revealed that VCAM1 Ab-treated mice had reduced positive area for galectin-3, a mannose receptor expressed by macrophages (FIG. 19C). This finding was supported by decreased hepatic mRNA expressions of the macrophage marker Cd68, the infiltrating proinflammatory monocyte marker Ccr2, and the proinflammatory cytokines Tnfα, and Il1b (FIG. 19D) in FFC-fed VCAM1 Ab-treated mice. These findings suggest that blockade of VCAM-1 reduces hepatic proinflammatory monocyte infiltration and MoMF-mediated liver inflammation.

### VCAM-1 Antibody Treatment in FFC-Fed Mice Reduces the Pro-Inflammatory Monocyte Hepatic Infiltration

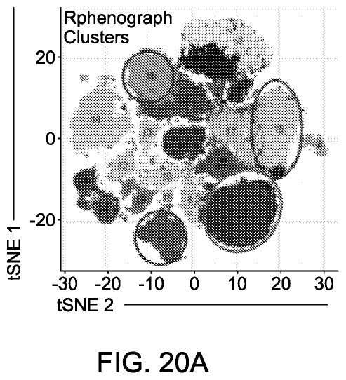

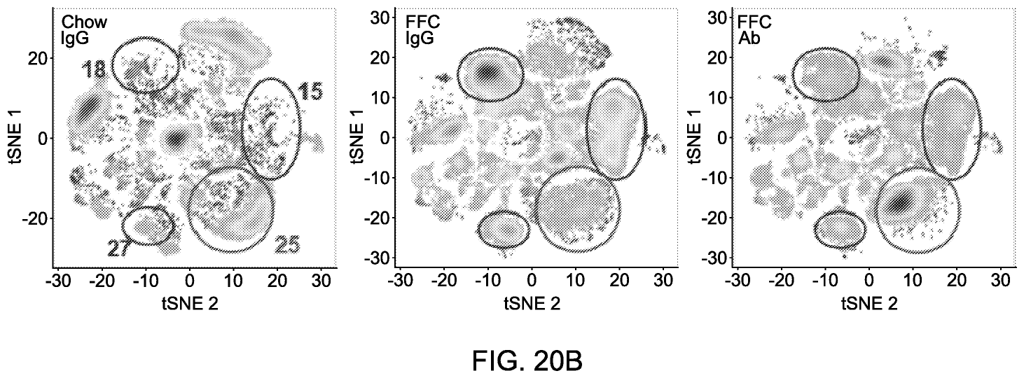

To examine the contribution of the different immune cells in the protective effect of VCAM1 Ab in NASH, mass cytometry by time-of-flight (CyTOF), which allows comprehensive profiling of the intrahepatic leukocyte subpopulations based on multiple cell surface markers, was utilized. Thirty-one clusters were obtained (FIG. 20A) based on the intensities of 30 different cell surface markers (Table 5) consisting of 23 leukocyte subset markers and 7 cell surface adhesion molecules. Out of 31 clusters obtained by CyTOF, 4 clusters were differentially expressed between the study groups (FIG. 20B) and categorized into distinct leukocyte subpopulations based on the intensities of individual cell surface markers. Clusters 18, 27, and 15 had typical expression markers of infiltrating proinflammatory macrophages, MoMFs, and hepatic macrophages, respectively (FIG. 20C). The abundance of all these three clusters was increased with the FFC-diet, but reduced with VCAM1 Ab treatment (FIG. 20B), suggesting that the protective effect of VCAM-1 blockade in diet-induced NASH is mainly through reduced proinflammatory monocyte recruitment and retention in the liver, which is consistent with whole liver mRNA expression of inflammatory genes (FIG. 19D). On the other hand, clusters 25 defined as B cells were unexpectedly reduced with the FFC-diet, and increased with VCAM1 Ab treatment (FIG. 20B). A potential explanation is that anti-inflammatory B cell population, also called “regulatory B cells (Bregs)”, is recruited and/or further differentiated from immature B cells to Bregs within the microenvironment of the VCAM1 Ab-treated mouse liver.

### Anti-VCAM-1 Antibody Treatment Reduces FFC Diet-Induced Liver Injury and Fibrosis in Murine NASH

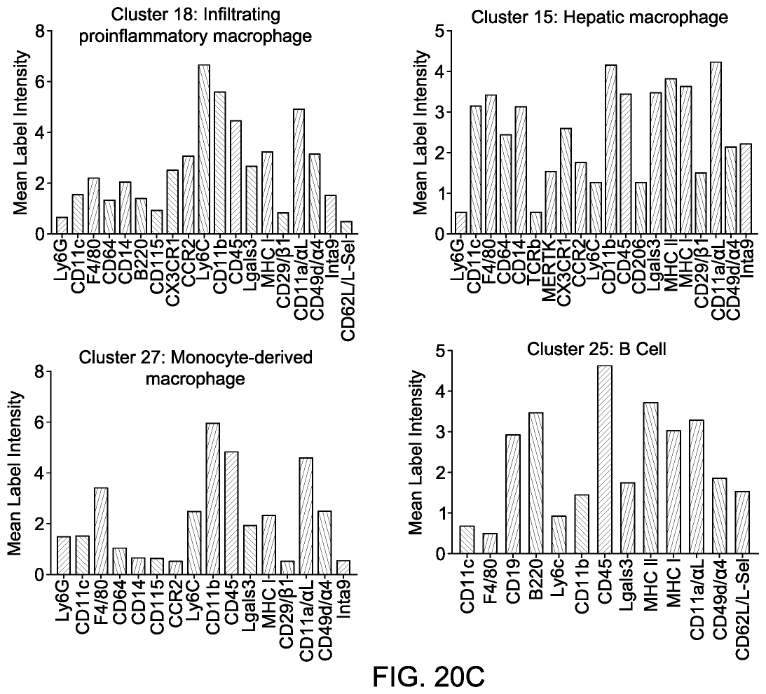

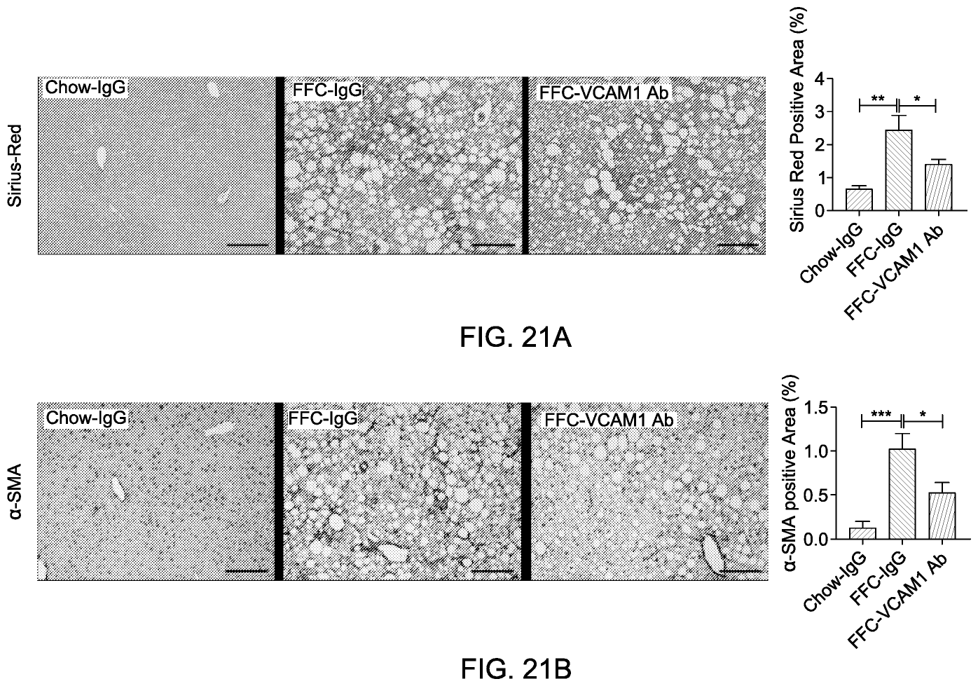

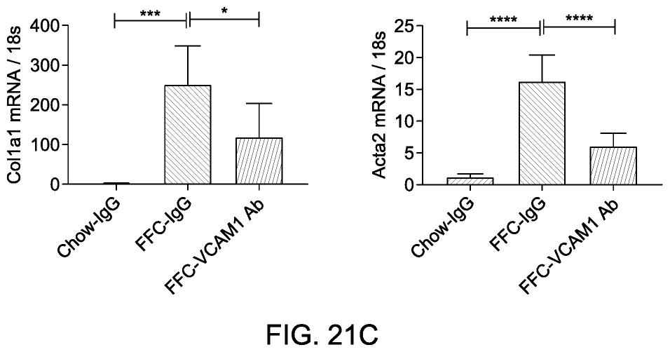

It was next examined whether reduced hepatic inflammation through VCAM-1 blockade may protect against stellate cell activation and liver fibrosis in a diet-induced NASH mouse model. Sirius red staining (FIG. 21A) as well as α-smooth muscle actin (α-SMA) immunohistochemistry (FIG. 21B) showed reduced pericellular fibrosis with VCAM1 Ab treatment. Likewise, mRNA levels of both Collagen 1α1 and Acta2 (α-SMA) were elevated in the FFC-fed mice, and significantly decreased with VCAM1 Ab treatment (FIG. 21C), further confirming the possible anti-fibrotic effect of VCAM1 Ab in NASH, likely through reduced liver inflammation. Collectively, these findings suggest that targeting VCAM-1 is a potential therapeutic approach to ameliorate hepatic inflammation, injury and fibrosis in human NASH.

### AGI-1067 Treatment in FFC-Fed Mice Attenuates Hepatic Injury, Inflammation and Fibrosis.

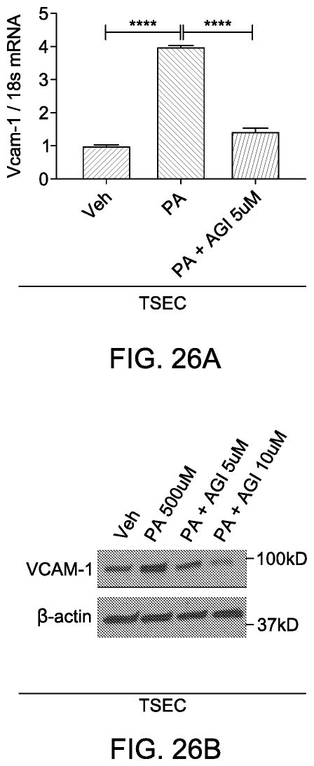

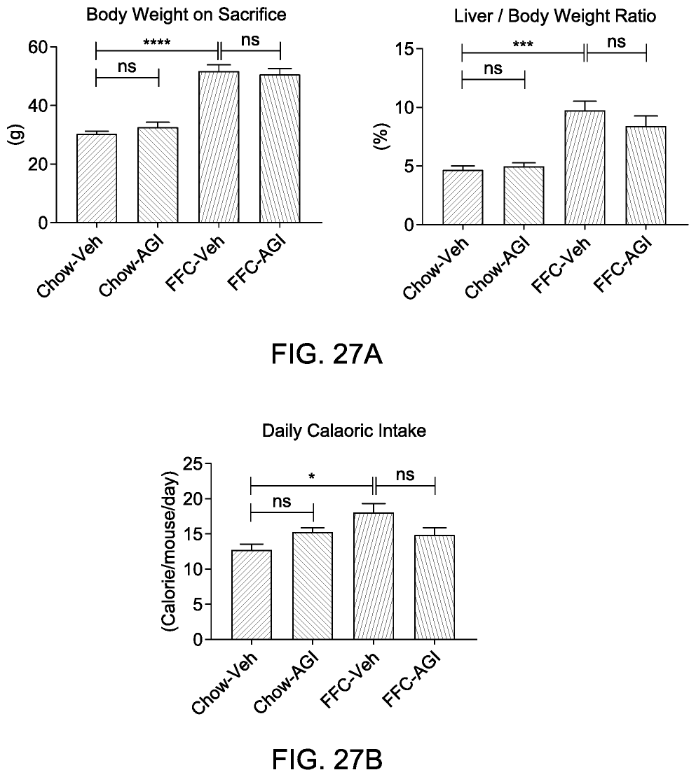

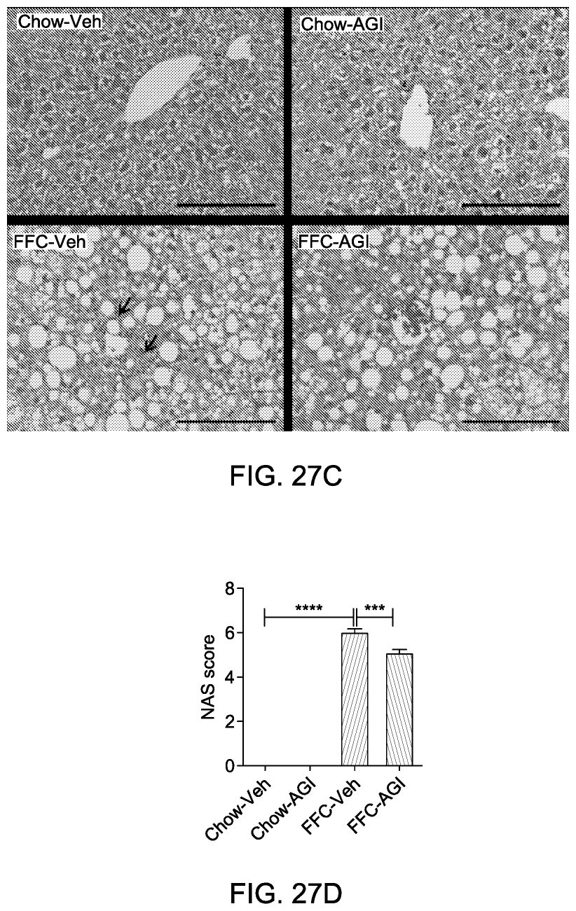

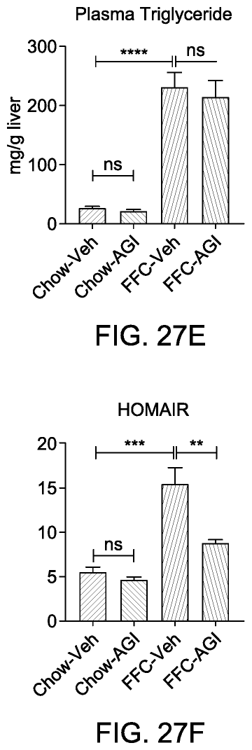

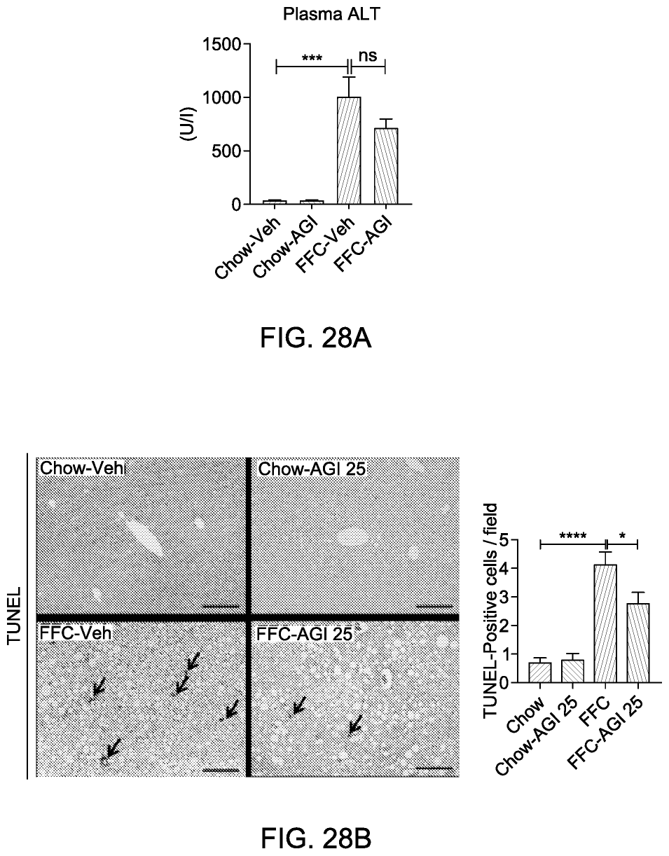

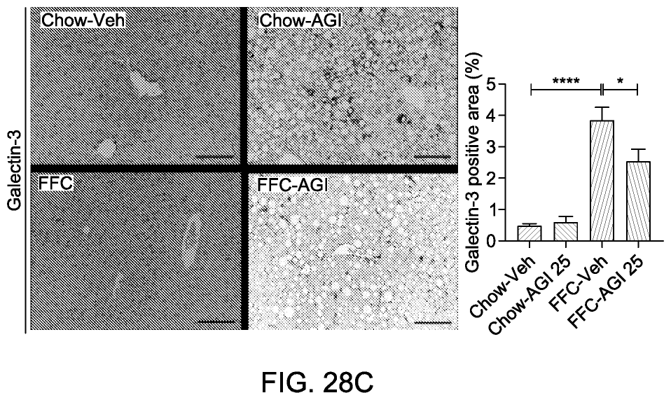

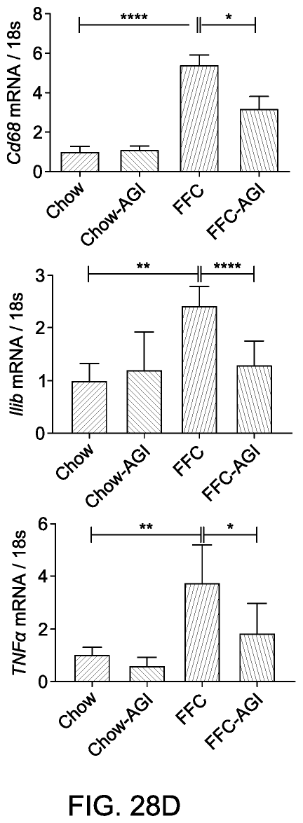

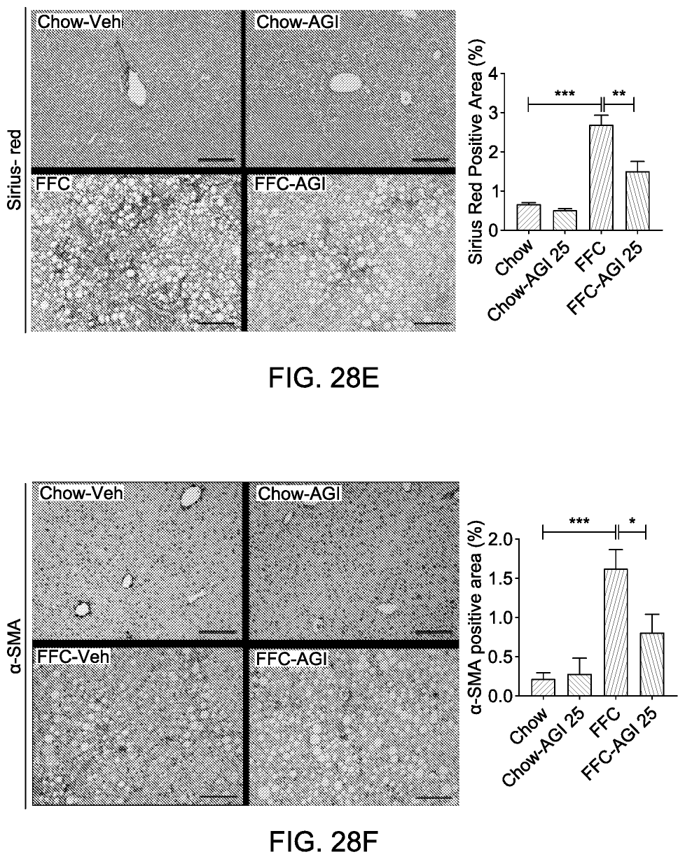

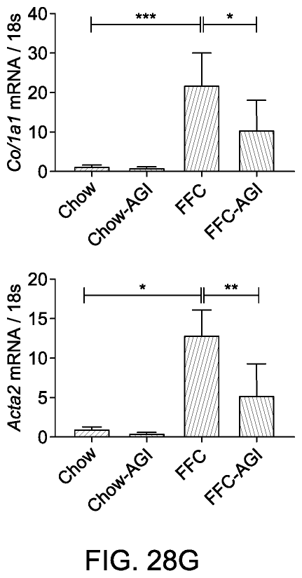

AGI-1067, also called succinobucol, has anti-oxidative and anti-inflammatory properties, with known VCAM-1 inhibitory function, and has been employed in clinical trials for atherosclerosis and type 2 diabetes. Thus, the effect of AGI-1067 was examined in a NASH mouse model. First, it was confirmed in vitro that AGI-1067 inhibits palmitate-induced VCAM-1 expression both at the mRNA and the protein levels (FIGS. 26A and B). In the diet-induced NASH model mice, AGI-1067 treatment did not alter weight gain, liver to body weight ratio, caloric intake, hepatic steatosis or liver triglyceride content in the FFC fed-mice (FIG. 27A-F). The NAS score was reduced in AGI-treated FFC-fed mice likely secondary to reduced liver inflammation (FIG. 27D). Although plasma ALT levels did not reach the statistical significance, AGI-1067-treated FFC-fed mice had reduced liver injury as assessed by the number of TUNEL positive hepatocyte (FIG. 28B), reduced MoMF-related hepatic inflammation as assessed by Galectin-3 staining of liver sections (FIG. 28C) and mRNA expressions of Cd68, Ccr2, and Tnfα (FIG. 28D), and attenuated stellate cell activation and liver fibrogenesis as assessed by Sirius red staining (FIG. 28E), immunohistochemistry for α-SMA (FIG. 28F) and mRNA expressions of Col1α1 and Acta2 (α-SMA) (FIG. 28G). Collectively, these findings are consistent with the observed protective effect of VCAM1 neutralizing Ab in FFC diet-induced mouse NASH.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating chronic liver disease in a mammal, wherein said method comprises administering an inhibitor of an integrin β1 (ITGβ1) polypeptide or an inhibitor of a vascular cell adhesion molecule 1 (VCAM-1) polypeptide to said mammal.

2. The method of claim 1, wherein said mammal is a human.

3. The method of claim 1, wherein said chronic liver disease is a non-alcoholic fatty liver disease.

4. The method of claim 3, wherein said non-alcoholic fatty liver disease is nonalcoholic steatohepatitis (NASH).

5. The method of claim 1, said method comprising administering an inhibitor of an ITGβ1 polypeptide to said mammal.

6. The method of claim 5, wherein said inhibitor of an ITGβ1 polypeptide is an ITGβ1 neutralizing antibody.

7-8. (canceled)

9. The method of claim 1, said method comprising administering an inhibitor of a VCAM-1 polypeptide to said mammal.

10. The method of claim 9, wherein said inhibitor of a VCAM-1 polypeptide is a VCAM-1 neutralizing antibody.

11. The method of claim 1, wherein said method comprises identifying said mammal as being in need of treatment of said chronic liver disease before said administering step.

12. A method for reducing liver inflammation in a mammal having chronic liver disease, wherein said method comprises administering an inhibitor of an integrin β1 (ITGβ1) polypeptide or an inhibitor of a vascular cell adhesion molecule 1 (VCAM-1) polypeptide to said mammal.

13. A method for reducing liver fibrosis in a mammal having chronic liver disease, wherein said method comprises administering an inhibitor of an integrin β1 (ITGβ1) polypeptide or an inhibitor of a vascular cell adhesion molecule 1 (VCAM-1) polypeptide to said mammal.

14. The method of claim 12, wherein said mammal is a human.

15. The method of claim 12, wherein said chronic liver disease is a non-alcoholic fatty liver disease.

16. The method of claim 15, wherein said non-alcoholic fatty liver disease is nonalcoholic steatohepatitis (NASH).

17. The method of claim 12, said method comprising administering an inhibitor of an ITGβ1 polypeptide to said mammal.

18. The method of claim 17, wherein said inhibitor of an ITGβ1 polypeptide is an ITGβ₁ neutralizing antibody.

19-20. (canceled)

21. The method of claim 12, said method comprising administering an inhibitor of a VCAM-1 polypeptide to said mammal.

22. The method of claim 21, wherein said inhibitor of a VCAM-1 polypeptide is a VCAM-1 neutralizing antibody.

23. The method of claim 12, wherein said method comprises identifying said mammal as being in need of said reduced liver inflammation or said reduced liver fibrosis before said administering step.
